SEQUENCE LISTING (989 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - DNA, 34 bp, Artificial sequence
  Synthetic
ctgtctcttatacacatctccgagcccacgagac

SEQ ID NO: 3 - DNA, 33 bp, Artificial sequence
  Synthetic
ctgtctcttatacacatctgacgctgccgacga

SEQ ID NO: 4 - DNA, 55 bp, Artificial sequence
  Synthetic
aaaaagaggaaggtggcggccgctggatccgagggcagaggaagtctgctaacat

SEQ ID NO: 5 - DNA, 33 bp, Artificial sequence
  Synthetic
aggttgattaccgataagcttgatatcgaattc

SEQ ID NO: 6 - DNA, 50 bp, Artificial sequence
  Synthetic
aagaggaaggtggcggccgctggatccatggacagcctcttgatgaaccg

SEQ ID NO: 7 - DNA, 52 bp, Artificial sequence
  Synthetic
ttcctctgccctctccactgcctgtacaaagtcccaaagtacgaaatgcgtc

SEQ ID NO: 8 - DNA, 52 bp, Artificial sequence
  Synthetic
ttcctctgccctctccactgcctgtacaagtacgaaatgcgtctcgtaagtc

SEQ ID NO: 9 - DNA, 43 bp, Artificial sequence
  Synthetic
gaacggctgccgcgtgcaattgctcttcctccgctacatctcg

SEQ ID NO: 10 - DNA, 43 bp, Artificial sequence
  Synthetic
aagagcaattgcacgcggcagccgttcttattgcgaagataac

SEQ ID NO: 11 - DNA, 69 bp, Artificial sequence
  Synthetic
aagaggaaggtggcggccgctggatccatggacagcctcttgatgaaccggagggagtttctttaccaa

SEQ ID NO: 12 - DNA, 56 bp, Artificial sequence
  Synthetic
tactgctggaatacttttgtagaaaaccacggaagaactttcaaagcctgggaagg

SEQ ID NO: 13 - DNA, 56 bp, Artificial sequence
  Synthetic
ccttcccaggctttgaaagttcttccgtggttttctacaaaagtattccagcagta

SEQ ID NO: 14 - DNA, 47 bp, Artificial sequence
  Synthetic
gctgctaccgcgtcacctggttcatctcctggagcccctgctacgac

SEQ ID NO: 15 - DNA, 47 bp, Artificial sequence
  Synthetic
gtcgtagcaggggctccaggagatgaaccaggtgacgcggtagcagc

SEQ ID NO: 16 - DNA, 49 bp, Artificial sequence
  Synthetic
catttcaggtgtcgtgagctagcccaccatggtgagcaagggcgaggag

SEQ ID NO: 17 - DNA, 53 bp, Artificial sequence
  Synthetic
ctggcttactagtcggttcaactctagattacttgtacagctcgtccatgccg

SEQ ID NO: 18 - DNA, 30 bp, Artificial sequence
  Synthetic
gtgaccaccttcagctacggcgtgcagtgc

SEQ ID NO: 19 - DNA, 30 bp, Artificial sequence
  Synthetic
gcactgcacgccgtagctgaaggtggtcac

SEQ ID NO: 20 - DNA, 38 bp, Artificial sequence
  Synthetic
accccgaccacatgaagcaccacgacttcttcaagtcc

SEQ ID NO: 21 - DNA, 38 bp, Artificial sequence
  Synthetic
ggacttgaagaagtcgtggtgcttcatgtggtcggggt

SEQ ID NO: 22 - DNA, 35 bp, Artificial sequence
  Synthetic
cctcgtgaccaccttcacctacggcgtgcagtgct

SEQ ID NO: 23 - DNA, 35 bp, Artificial sequence
  Synthetic
agcactgcacgccgtaggtgaaggtggtcacgagg

SEQ ID NO: 24 - DNA, 52 bp, Artificial sequence
  Synthetic
tttcttccatttcaggtgtcgtgatgtacaatgaccgagtacaagcccacgg

SEQ ID NO: 25 - DNA, 52 bp, Artificial sequence
  Synthetic
attaccgataagcttgatatcgaattctcaggcaccgggcttgcgggtcatg

SEQ ID NO: 26 - DNA, 30 bp, Artificial sequence
  Synthetic
tcctggccaccgtcggcgtatcgcccgacc

SEQ ID NO: 27 - DNA, 30 bp, Artificial sequence
  Synthetic
ggtcgggcgatacgccgacggtggccagga

SEQ ID NO: 28 - DNA, 20 bp, Artificial sequence
  Synthetic
ggcgagggcgatgccaccta

SEQ ID NO: 29 - DNA, 23 bp, Artificial sequence
  Synthetic
gctcaagaacgccttccccagtc

SEQ ID NO: 30 - DNA, 19 bp, Artificial sequence
  Synthetic
ggcacgggcagcttgccgg

SEQ ID NO: 31 - DNA, 19 bp, Artificial sequence
  Synthetic
aagggcatcgacttcaagg

SEQ ID NO: 32 - DNA, 20 bp, Artificial sequence
  Synthetic
cgatgcccttcagctcgatg

SEQ ID NO: 33 - DNA, 20 bp, Artificial sequence
  Synthetic
ctcgtgaccaccctgaccta

SEQ ID NO: 34 - DNA, 20 bp, Artificial sequence
  Synthetic
caagttcagcgtgtctggcg

SEQ ID NO: 35 - DNA, 20 bp, Artificial sequence
  Synthetic
caactacaagacccgcgccg

SEQ ID NO: 36 - DNA, 20 bp, Artificial sequence
  Synthetic
ggtgaaccgcatcgagctga

SEQ ID NO: 37 - DNA, 20 bp, Artificial sequence
  Synthetic
cggccatgatatagacgttg

SEQ ID NO: 38 - DNA, 20 bp, Artificial sequence
  Synthetic
cgtcgccgtccagctcgacc

SEQ ID NO: 39 - DNA, 20 bp, Artificial sequence
  Synthetic
agcactgcacgccgtaggtc

SEQ ID NO: 40 - DNA, 20 bp, Artificial sequence
  Synthetic
tcagctcgatgcggttcacc

SEQ ID NO: 41 - DNA, 20 bp, Artificial sequence
  Synthetic
ccggcaagctgcccgtgccc

SEQ ID NO: 42 - DNA, 20 bp, Artificial sequence
  Synthetic
gcttcatgtggtcggggtag

SEQ ID NO: 43 - DNA, 20 bp, Artificial sequence
  Synthetic
cgtgctgcttcatgtggtcg

SEQ ID NO: 44 - DNA, 20 bp, Artificial sequence
  Synthetic
gtcgtgctgcttcatgtggt

SEQ ID NO: 45 - DNA, 17 bp, Artificial sequence
  Synthetic
tccccctcagccgtatt

SEQ ID NO: 46 - DNA, 17 bp, Artificial sequence
  Synthetic
gattgatattgccttct

SEQ ID NO: 47 - DNA, 17 bp, Artificial sequence
  Synthetic
tctgactcctaatggag

SEQ ID NO: 48 - DNA, 17 bp, Artificial sequence
  Synthetic
attactttagagtaaga

SEQ ID NO: 49 - DNA, 19 bp, Artificial sequence
  Synthetic
ggtccatgggtagacaacc

SEQ ID NO: 50 - DNA, 20 bp, Artificial sequence
  Synthetic
gtgagattgacaagaacagt

SEQ ID NO: 51 - DNA, 20 bp, Artificial sequence
  Synthetic
aggtcgcttctcaggatttg

SEQ ID NO: 52 - DNA, 20 bp, Artificial sequence
  Synthetic
gagatcatccaggtgctttg

SEQ ID NO: 53 - DNA, 20 bp, Artificial sequence
  Synthetic
gctactatcacaagcctgtg

SEQ ID NO: 54 - DNA, 19 bp, Artificial sequence
  Synthetic
ggagatgtatttgcagcgg

SEQ ID NO: 55 - DNA, 20 bp, Artificial sequence
  Synthetic
ggacatggtgaatgacggcg

SEQ ID NO: 56 - DNA, 20 bp, Artificial sequence
  Synthetic
agccacctgaggggtaaggg

SEQ ID NO: 57 - DNA, 20 bp, Artificial sequence
  Synthetic
ctgcaccctgacccaagaag

SEQ ID NO: 58 - DNA, 20 bp, Artificial sequence
  Synthetic
tgatcaggcgcccagtcacg

SEQ ID NO: 59 - DNA, 17 bp, Artificial sequence
  Synthetic
gccgaggagaagcgcga

SEQ ID NO: 60 - DNA, 20 bp, Artificial sequence
  Synthetic
gcgcgaggagccttgatttg

SEQ ID NO: 61 - DNA, 20 bp, Artificial sequence
  Synthetic
ctctatttccagcggttaag

SEQ ID NO: 62 - DNA, 20 bp, Artificial sequence
  Synthetic
tagcgggaggcgaggccaag

SEQ ID NO: 63 - DNA, 20 bp, Artificial sequence
  Synthetic
acgcgccagccttctttgtg

SEQ ID NO: 64 - DNA, 20 bp, Artificial sequence
  Synthetic
gtttgttcttagggtaacag

SEQ ID NO: 65 - DNA, 20 bp, Artificial sequence
  Synthetic
tatccttgtgaagctaggag

SEQ ID NO: 66 - DNA, 20 bp, Artificial sequence
  Synthetic
tgttcttggcgctactgatg

SEQ ID NO: 67 - DNA, 20 bp, Artificial sequence
  Synthetic
ggcgagtgtgtatagatcag

SEQ ID NO: 68 - DNA, 20 bp, Artificial sequence
  Synthetic
taatgcatgttgttagggag

SEQ ID NO: 69 - DNA, 20 bp, Artificial sequence
  Synthetic
tggggagttagtatactggg

SEQ ID NO: 70 - DNA, 20 bp, Artificial sequence
  Synthetic
atacacactatagtggactg

SEQ ID NO: 71 - DNA, 20 bp, Artificial sequence
  Synthetic
aactcccacagcatttatcc

SEQ ID NO: 72 - DNA, 20 bp, Artificial sequence
  Synthetic
atgggaaaatgaatggctga

SEQ ID NO: 73 - DNA, 20 bp, Artificial sequence
  Synthetic
caccaccaattccaagagag

SEQ ID NO: 74 - DNA, 20 bp, Artificial sequence
  Synthetic
caatgcaggctggttctcag

SEQ ID NO: 75 - DNA, 20 bp, Artificial sequence
  Synthetic
tttcatagccgggaaacctg

SEQ ID NO: 76 - DNA, 20 bp, Artificial sequence
  Synthetic
tcagggagggggaccgtaac

SEQ ID NO: 77 - DNA, 20 bp, Artificial sequence
  Synthetic
ggagggggaccgtaacagga

SEQ ID NO: 78 - DNA, 20 bp, Artificial sequence
  Synthetic
attcagggacttggatttgg

SEQ ID NO: 79 - DNA, 20 bp, Artificial sequence
  Synthetic
cctcatctgttggcaccaag

SEQ ID NO: 80 - DNA, 20 bp, Artificial sequence
  Synthetic
aggagagagcaacgtgcaag

SEQ ID NO: 81 - DNA, 20 bp, Artificial sequence
  Synthetic
gcggtctgggtgccctcgta

SEQ ID NO: 82 - DNA, 22 bp, Artificial sequence
  Synthetic
aggccagcttggcacttgatgt

SEQ ID NO: 83 - DNA, 23 bp, Artificial sequence
  Synthetic
tgttgtggcggatcttgaagttc

SEQ ID NO: 84 - DNA, 22 bp, Artificial sequence
  Synthetic
aggccagcttggcacttgatgt

SEQ ID NO: 85 - DNA, 22 bp, Artificial sequence
  Synthetic
gcttcagcctctgcttgatctc

SEQ ID NO: 86 - DNA, 22 bp, Artificial sequence
  Synthetic
cactatgaccacagccactcac

SEQ ID NO: 87 - DNA, 25 bp, Artificial sequence
  Synthetic
ctttctgaaaagtaacccagcctca

SEQ ID NO: 88 - DNA, 28 bp, Artificial sequence
  Synthetic
gaactgtgaataataagcaatcatccag

SEQ ID NO: 89 - DNA, 27 bp, Artificial sequence
  Synthetic
gcttgccaaaaattgtgtaccctttcc

SEQ ID NO: 90 - DNA, 29 bp, Artificial sequence
  Synthetic
taggtaacccatctgaggttttcaaatat

SEQ ID NO: 91 - DNA, 26 bp, Artificial sequence
  Synthetic
gagaaaagaacatgacttccagcagc

SEQ ID NO: 92 - DNA, 26 bp, Artificial sequence
  Synthetic
ccaaattgcagccacacttgaaaacc

SEQ ID NO: 93 - DNA, 24 bp, Artificial sequence
  Synthetic
taggaagcagtgtaggaggattgg

SEQ ID NO: 94 - DNA, 22 bp, Artificial sequence
  Synthetic
aggccagcttggcacttgatgt

SEQ ID NO: 95 - DNA, 22 bp, Artificial sequence
  Synthetic
aagcagcgtatccacatagcgt

SEQ ID NO: 96 - DNA, 21 bp, Artificial sequence
  Synthetic
gcaagggggctggctccacac

SEQ ID NO: 97 - DNA, 25 bp, Artificial sequence
  Synthetic
ttagttctttctgcccacactagac

SEQ ID NO: 98 - DNA, 23 bp, Artificial sequence
  Synthetic
catgtggttgcagcttaactcac

SEQ ID NO: 99 - DNA, 24 bp, Artificial sequence
  Synthetic
gtgtttttgtggtcttatgtggcc

SEQ ID NO: 100 - DNA, 23 bp, Artificial sequence
  Synthetic
acaacataccaccccatctcacc

SEQ ID NO: 101 - DNA, 24 bp, Artificial sequence
  Synthetic
caaagtgctgggattacgggtttg

SEQ ID NO: 102 - DNA, 23 bp, Artificial sequence
  Synthetic
caagcagctgcatccaccctctt

SEQ ID NO: 103 - DNA, 25 bp, Artificial sequence
  Synthetic
ctgctaacctcatctccctttccag

SEQ ID NO: 104 - DNA, 23 bp, Artificial sequence
  Synthetic
gtatcttcaaacagctcacaccc

SEQ ID NO: 105 - DNA, 23 bp, Artificial sequence
  Synthetic
gtcttagagtatccagtgaggcc

SEQ ID NO: 106 - DNA, 23 bp, Artificial sequence
  Synthetic
cactgaggttacgtagtttgccc

SEQ ID NO: 107 - DNA, 22 bp, Artificial sequence
  Synthetic
cgacaaatcctcctccacctct

SEQ ID NO: 108 - DNA, 22 bp, Artificial sequence
  Synthetic
ttcctctccgcttgcaacctcc

SEQ ID NO: 109 - DNA, 27 bp, Artificial sequence
  Synthetic
cggcacatagaactaaacctacatttc

SEQ ID NO: 110 - DNA, 29 bp, Artificial sequence
  Synthetic
gccagtaagcattttcctaatagatggac

SEQ ID NO: 111 - DNA, 23 bp, Artificial sequence
  Synthetic
gccaaatgccaagagtttaagcc

SEQ ID NO: 112 - DNA, 26 bp, Artificial sequence
  Synthetic
tcatccttctgaactcaattgctttg

SEQ ID NO: 113 - DNA, 30 bp, Artificial sequence
  Synthetic
caatgatgcaaatgctcttaaaagaaactc

SEQ ID NO: 114 - DNA, 27 bp, Artificial sequence
  Synthetic
ggtgactatttcatttgtgtgacactc

SEQ ID NO: 115 - DNA, 23 bp, Artificial sequence
  Synthetic
gaaagcagtgttcagggtctacc

SEQ ID NO: 116 - DNA, 24 bp, Artificial sequence
  Synthetic
gaaaacctgaacaaatggagaggg

SEQ ID NO: 117 - DNA, 29 bp, Artificial sequence
  Synthetic
gcttgctcagtagattataatcctacagg

SEQ ID NO: 118 - DNA, 23 bp, Artificial sequence
  Synthetic
ggtcgataagtcttccgaacctc

SEQ ID NO: 119 - DNA, 28 bp, Artificial sequence
  Synthetic
gcgaaactggtgagttactaattaatcc

SEQ ID NO: 120 - DNA, 20 bp, Artificial sequence
  Synthetic
ccgcgctggttcaccggtag

SEQ ID NO: 121 - DNA, 20 bp, Artificial sequence
  Synthetic
ctgcaactatgactccatgg

SEQ ID NO: 122 - DNA, 20 bp, Artificial sequence
  Synthetic
tcatagttgcagctgactcc

SEQ ID NO: 123 - DNA, 20 bp, Artificial sequence
  Synthetic
agctgactccagggctacag

SEQ ID NO: 124 - DNA, 20 bp, Artificial sequence
  Synthetic
ctgctaggcaccatggctgg

SEQ ID NO: 125 - DNA, 20 bp, Artificial sequence
  Synthetic
caacctctaccacgtgcggg

SEQ ID NO: 126 - DNA, 20 bp, Artificial sequence
  Synthetic
tgaagggaaccggatttcag

SEQ ID NO: 127 - DNA, 100 bp, Artificial sequence
  Synthetic
cagatctgcacgacccccaagtccgaaaaacccgcgctggttcaccggtaacggtctctccaacacgctggcaagcgccatgtctagtgtgggcagaaag

SEQ ID NO: 128 - DNA, 101 bp, Artificial sequence
  Synthetic
ctccctggacctagatccagcagatctgcacgacccccaagtccgaaaaatccgcgctggttcaccggtagcggtctctccaacacgctggcaagcgccat

SEQ ID NO: 129 - DNA, 101 bp, Artificial sequence
  Synthetic
acccgctgtagccctggagtcagctgcaactatgactccatggcggaactattaagatcagaggaaaacacaaaacaggccacataagaccacaaaaacac

SEQ ID NO: 130 - DNA, 101 bp, Artificial sequence
  Synthetic
ctatcaccttcttcaccgtctgggaggcaatgtaagcacccgctgtagccttggagtcagctgcaactatgactccatggcggaactgttaagatcagagg

SEQ ID NO: 131 - DNA, 101 bp, Artificial sequence
  Synthetic
ctctatcaccttcttcaccgtctgggaggcaatgtaagcacccgctgtagtcctggagtcagctgcaactatgactccatggcggaactgttaagatcaga

SEQ ID NO: 132 - DNA, 101 bp, Artificial sequence
  Synthetic
tctctatcaccttcttcaccgtctgggaggcaatgtaagcacccgctgtacccctggagtcagctgcaactatgactccatggcggaactgttaagatcag

SEQ ID NO: 133 - DNA, 101 bp, Artificial sequence
  Synthetic
atgggttgatctctatcaccttcttcaccgtctgggaggcaatgtaagcatccgctgtagccctggagtcagctgcaactatgactccatggcggaactgt

SEQ ID NO: 134 - DNA, 101 bp, Artificial sequence
  Synthetic
agattcgacattgccgagccaacagccgttcccagaagctgcaatccgctacgcccccagccatggtgcctagcaggtatgggttgatctctatcaccttc

SEQ ID NO: 135 - DNA, 101 bp, Artificial sequence
  Synthetic
atctctatcaccttcttcaccgtctgggaggcaatgtaagcacccgctgtcgccctggagtcagctgcaactatgactccatggcggaactgttaagatca

SEQ ID NO: 136 - DNA, 101 bp, Artificial sequence
  Synthetic
tatacttctcatgtagatcagccacattgtcactggagactcggatccagtcatcctcccgcacgtggtagaggttgactgcacctcctgagtaggcatct

SEQ ID NO: 137 - DNA, 101 bp, Artificial sequence
  Synthetic
tccatgaccccatatgcatacacagagccagaacctacagagaaggtggcacctgaaatccggttcccttcactgtccacgtagtagaggcctggaaaggg

SEQ ID NO: 138 - DNA, 20 bp, Artificial sequence
  Synthetic
ggctaaattcctcttattca

SEQ ID NO: 139 - DNA, 20 bp, Artificial sequence
  Synthetic
gtaaccaagagtcaggactg

SEQ ID NO: 140 - DNA, 20 bp, Artificial sequence
  Synthetic
gggataatataaggcattct

SEQ ID NO: 141 - DNA, 20 bp, Artificial sequence
  Synthetic
ggatcttataatctagttat

SEQ ID NO: 142 - DNA, 20 bp, Artificial sequence
  Synthetic
gttaatgccttggtcaaatg

SEQ ID NO: 143 - DNA, 20 bp, Artificial sequence
  Synthetic
gtgtaaactaagacctaagt

SEQ ID NO: 144 - DNA, 20 bp, Artificial sequence
  Synthetic
gctaaagttgtcattgattt

SEQ ID NO: 145 - DNA, 20 bp, Artificial sequence
  Synthetic
gtgcttccgacaaactacaa

SEQ ID NO: 146 - DNA, 20 bp, Artificial sequence
  Synthetic
ggaacgtaggtaataaggtc

SEQ ID NO: 147 - DNA, 20 bp, Artificial sequence
  Synthetic
gattcttcatatctttctca

SEQ ID NO: 148 - DNA, 20 bp, Artificial sequence
  Synthetic
gctcatgagacacttcacag

SEQ ID NO: 149 - DNA, 20 bp, Artificial sequence
  Synthetic
gtcagcattaaacatgctta

SEQ ID NO: 150 - DNA, 20 bp, Artificial sequence
  Synthetic
gtgaaagttctcatcttctt

SEQ ID NO: 151 - DNA, 20 bp, Artificial sequence
  Synthetic
gcatgagaagaggagattga

SEQ ID NO: 152 - DNA, 20 bp, Artificial sequence
  Synthetic
gactgttcataggaccctaa

SEQ ID NO: 153 - DNA, 20 bp, Artificial sequence
  Synthetic
gccctgtctgtatccagtcc

SEQ ID NO: 154 - DNA, 20 bp, Artificial sequence
  Synthetic
gggatctttcagtgtaggta

SEQ ID NO: 155 - DNA, 20 bp, Artificial sequence
  Synthetic
gattctgtataatggaaatc

SEQ ID NO: 156 - DNA, 20 bp, Artificial sequence
  Synthetic
gacatgtcctaattgtatgg

SEQ ID NO: 157 - DNA, 20 bp, Artificial sequence
  Synthetic
gtgtgctttgaagaataatg

SEQ ID NO: 158 - DNA, 20 bp, Artificial sequence
  Synthetic
gcaatatgatctcatttgtg

SEQ ID NO: 159 - DNA, 20 bp, Artificial sequence
  Synthetic
gagtttagaggtttgagatt

SEQ ID NO: 160 - DNA, 20 bp, Artificial sequence
  Synthetic
gtggtcctggactggtctca

SEQ ID NO: 161 - DNA, 20 bp, Artificial sequence
  Synthetic
gttatgccaacacatttgta

SEQ ID NO: 162 - DNA, 20 bp, Artificial sequence
  Synthetic
gttacatacaaaaattggat

SEQ ID NO: 163 - DNA, 20 bp, Artificial sequence
  Synthetic
gcatattatcactccagtga

SEQ ID NO: 164 - DNA, 20 bp, Artificial sequence
  Synthetic
gacattgggattaaatttgg

SEQ ID NO: 165 - DNA, 20 bp, Artificial sequence
  Synthetic
ggtggccgccatcatggctg

SEQ ID NO: 166 - DNA, 20 bp, Artificial sequence
  Synthetic
ggcagatcagaatgtgagct

SEQ ID NO: 167 - DNA, 20 bp, Artificial sequence
  Synthetic
gaggaaggagttatattgac

SEQ ID NO: 168 - DNA, 20 bp, Artificial sequence
  Synthetic
gagccaaagataagcatgag

SEQ ID NO: 169 - DNA, 20 bp, Artificial sequence
  Synthetic
ggctactcagatatagtcat

SEQ ID NO: 170 - DNA, 20 bp, Artificial sequence
  Synthetic
gttatttgatgagcagctat

SEQ ID NO: 171 - DNA, 20 bp, Artificial sequence
  Synthetic
gacgtagtaaggtagagaca

SEQ ID NO: 172 - DNA, 20 bp, Artificial sequence
  Synthetic
gtgatgaagagtgctacagc

SEQ ID NO: 173 - DNA, 20 bp, Artificial sequence
  Synthetic
gctagggacttcaaagttat

SEQ ID NO: 174 - DNA, 20 bp, Artificial sequence
  Synthetic
gatatcttcccaatgatgac

SEQ ID NO: 175 - DNA, 20 bp, Artificial sequence
  Synthetic
gagtagtttctgacgtccga

SEQ ID NO: 176 - DNA, 20 bp, Artificial sequence
  Synthetic
gagcataatgaaggttcttg

SEQ ID NO: 177 - DNA, 20 bp, Artificial sequence
  Synthetic
gcgtttccaatcccagagag

SEQ ID NO: 178 - DNA, 20 bp, Artificial sequence
  Synthetic
ggcctaatagctttggtaga

SEQ ID NO: 179 - DNA, 20 bp, Artificial sequence
  Synthetic
gacaggaggaacttgtaacc

SEQ ID NO: 180 - DNA, 20 bp, Artificial sequence
  Synthetic
gagagcactcagcaaaatca

SEQ ID NO: 181 - DNA, 20 bp, Artificial sequence
  Synthetic
gcgttggtgaaattacaatt

SEQ ID NO: 182 - DNA, 20 bp, Artificial sequence
  Synthetic
gttaatgatcaaaagttaca

SEQ ID NO: 183 - DNA, 20 bp, Artificial sequence
  Synthetic
gagagaattgctattctgag

SEQ ID NO: 184 - DNA, 20 bp, Artificial sequence
  Synthetic
gattgtatgaaaacatagat

SEQ ID NO: 185 - DNA, 20 bp, Artificial sequence
  Synthetic
ggctacctgtctattggcac

SEQ ID NO: 186 - DNA, 20 bp, Artificial sequence
  Synthetic
ggcatgtgtgtctgaataca

SEQ ID NO: 187 - DNA, 20 bp, Artificial sequence
  Synthetic
gctgaagctctggcaagagc

SEQ ID NO: 188 - DNA, 20 bp, Artificial sequence
  Synthetic
gtaccttaatcacacctttg

SEQ ID NO: 189 - DNA, 20 bp, Artificial sequence
  Synthetic
gttcacatagcagtacttgt

SEQ ID NO: 190 - DNA, 20 bp, Artificial sequence
  Synthetic
gactgacctttctttgagag

SEQ ID NO: 191 - DNA, 20 bp, Artificial sequence
  Synthetic
gacttgaatgatcaattact

SEQ ID NO: 192 - DNA, 20 bp, Artificial sequence
  Synthetic
gttctgagttactggaaccc

SEQ ID NO: 193 - DNA, 20 bp, Artificial sequence
  Synthetic
gcaagatcaggtaagtatct

SEQ ID NO: 194 - DNA, 20 bp, Artificial sequence
  Synthetic
gtcgtgaagctgtgtttgac

SEQ ID NO: 195 - DNA, 20 bp, Artificial sequence
  Synthetic
ggtcttgaaataaaatttag

SEQ ID NO: 196 - DNA, 20 bp, Artificial sequence
  Synthetic
gactgcttcttagttaggta

SEQ ID NO: 197 - DNA, 20 bp, Artificial sequence
  Synthetic
ggaaatccttgagtttcagg

SEQ ID NO: 198 - DNA, 20 bp, Artificial sequence
  Synthetic
gcccaagcaggctacattgc

SEQ ID NO: 199 - DNA, 20 bp, Artificial sequence
  Synthetic
gaggtggcaaagaatgtgcc

SEQ ID NO: 200 - DNA, 20 bp, Artificial sequence
  Synthetic
gttcaaataatagggtgcat

SEQ ID NO: 201 - DNA, 20 bp, Artificial sequence
  Synthetic
gaggggatactcaagctagg

SEQ ID NO: 202 - DNA, 20 bp, Artificial sequence
  Synthetic
gggtatcagctcacctcctc

SEQ ID NO: 203 - DNA, 20 bp, Artificial sequence
  Synthetic
gaagtactggcaatgcaact

SEQ ID NO: 204 - DNA, 20 bp, Artificial sequence
  Synthetic
gacatagcctgcaattgttt

SEQ ID NO: 205 - DNA, 20 bp, Artificial sequence
  Synthetic
gggcagattggaagagccct

SEQ ID NO: 206 - DNA, 20 bp, Artificial sequence
  Synthetic
gtgtacaacatcacagcata

SEQ ID NO: 207 - DNA, 20 bp, Artificial sequence
  Synthetic
gggtggttctgaatgggagc

SEQ ID NO: 208 - DNA, 20 bp, Artificial sequence
  Synthetic
gctatccttaaattggcctg

SEQ ID NO: 209 - DNA, 20 bp, Artificial sequence
  Synthetic
gcctgaatatagtgaaagtc

SEQ ID NO: 210 - DNA, 20 bp, Artificial sequence
  Synthetic
gggaagtcctggggtttgat

SEQ ID NO: 211 - DNA, 20 bp, Artificial sequence
  Synthetic
gtcagttattctttcctcta

SEQ ID NO: 212 - DNA, 20 bp, Artificial sequence
  Synthetic
gcatggtcacaataatcttg

SEQ ID NO: 213 - DNA, 20 bp, Artificial sequence
  Synthetic
gggaggataagagacacttt

SEQ ID NO: 214 - DNA, 20 bp, Artificial sequence
  Synthetic
gcttatttagtttggttcaa

SEQ ID NO: 215 - DNA, 20 bp, Artificial sequence
  Synthetic
gtctctactagaactcaatc

SEQ ID NO: 216 - DNA, 20 bp, Artificial sequence
  Synthetic
ggagcttggtatctaaaatt

SEQ ID NO: 217 - DNA, 20 bp, Artificial sequence
  Synthetic
gatgttcactgttaattgat

SEQ ID NO: 218 - DNA, 20 bp, Artificial sequence
  Synthetic
gctacttaaatcattgccat

SEQ ID NO: 219 - DNA, 20 bp, Artificial sequence
  Synthetic
gcacttcacctgagaaaaac

SEQ ID NO: 220 - DNA, 20 bp, Artificial sequence
  Synthetic
gcttgcttgtctctgtttcg

SEQ ID NO: 221 - DNA, 20 bp, Artificial sequence
  Synthetic
gtcaacagcaaggctactga

SEQ ID NO: 222 - DNA, 20 bp, Artificial sequence
  Synthetic
gacagaagaagctagaagtc

SEQ ID NO: 223 - DNA, 20 bp, Artificial sequence
  Synthetic
gtacaacccaaagtatatgg

SEQ ID NO: 224 - DNA, 20 bp, Artificial sequence
  Synthetic
gaatcccgggctttctctgt

SEQ ID NO: 225 - DNA, 20 bp, Artificial sequence
  Synthetic
gataatttcaggagtgagat

SEQ ID NO: 226 - DNA, 20 bp, Artificial sequence
  Synthetic
gtattgtgatcaagtaattt

SEQ ID NO: 227 - DNA, 20 bp, Artificial sequence
  Synthetic
gaacctaaaaatatagttgt

SEQ ID NO: 228 - DNA, 20 bp, Artificial sequence
  Synthetic
gcattggtgcccagtaggag

SEQ ID NO: 229 - DNA, 20 bp, Artificial sequence
  Synthetic
gaatactgtgagaaatttca

SEQ ID NO: 230 - DNA, 20 bp, Artificial sequence
  Synthetic
gtcaagatatacctagcaaa

SEQ ID NO: 231 - DNA, 20 bp, Artificial sequence
  Synthetic
gacctcacttactgttgcca

SEQ ID NO: 232 - DNA, 20 bp, Artificial sequence
  Synthetic
gcataccatagggtaaaggc

SEQ ID NO: 233 - DNA, 20 bp, Artificial sequence
  Synthetic
ggtgacaatcaaactggcaa

SEQ ID NO: 234 - DNA, 20 bp, Artificial sequence
  Synthetic
ggtattgtcaatgtaaaaag

SEQ ID NO: 235 - DNA, 20 bp, Artificial sequence
  Synthetic
gcacagtaaatatacgtgtg

SEQ ID NO: 236 - DNA, 20 bp, Artificial sequence
  Synthetic
gtgtgcccctccaaaagaga

SEQ ID NO: 237 - DNA, 20 bp, Artificial sequence
  Synthetic
gacatatgctatgcagagtt

SEQ ID NO: 238 - DNA, 20 bp, Artificial sequence
  Synthetic
gtaagaatcaaatcatcatg

SEQ ID NO: 239 - DNA, 20 bp, Artificial sequence
  Synthetic
ggaaattgcttctggtttat

SEQ ID NO: 240 - DNA, 20 bp, Artificial sequence
  Synthetic
gtagatgagctcttatcagt

SEQ ID NO: 241 - DNA, 20 bp, Artificial sequence
  Synthetic
ggctttgttcatgactttga

SEQ ID NO: 242 - DNA, 20 bp, Artificial sequence
  Synthetic
gcaccagtctatgccaccac

SEQ ID NO: 243 - DNA, 20 bp, Artificial sequence
  Synthetic
gtaatgacttgggggagata

SEQ ID NO: 244 - DNA, 20 bp, Artificial sequence
  Synthetic
gagtctgtctctaatgagac

SEQ ID NO: 245 - DNA, 20 bp, Artificial sequence
  Synthetic
gtggtccacagacaatgcat

SEQ ID NO: 246 - DNA, 20 bp, Artificial sequence
  Synthetic
ggttaagaaaagacactcag

SEQ ID NO: 247 - DNA, 20 bp, Artificial sequence
  Synthetic
ggtaatcataagttgtataa

SEQ ID NO: 248 - DNA, 20 bp, Artificial sequence
  Synthetic
ggccctccttagaagttgca

SEQ ID NO: 249 - DNA, 20 bp, Artificial sequence
  Synthetic
gaaattggtccccaccttca

SEQ ID NO: 250 - DNA, 20 bp, Artificial sequence
  Synthetic
gtccaagaacaaagcaaaga

SEQ ID NO: 251 - DNA, 20 bp, Artificial sequence
  Synthetic
gatgagccaatctttagcaa

SEQ ID NO: 252 - DNA, 20 bp, Artificial sequence
  Synthetic
gtgaatcaagaagcaatgtc

SEQ ID NO: 253 - DNA, 20 bp, Artificial sequence
  Synthetic
gaaaggcagacatggctaaa

SEQ ID NO: 254 - DNA, 20 bp, Artificial sequence
  Synthetic
gacaaaagcagaataccaga

SEQ ID NO: 255 - DNA, 20 bp, Artificial sequence
  Synthetic
gcacacaaaatatcgttatt

SEQ ID NO: 256 - DNA, 20 bp, Artificial sequence
  Synthetic
gagaaaggcccagctctgat

SEQ ID NO: 257 - DNA, 20 bp, Artificial sequence
  Synthetic
gccagtctacccactgtccc

SEQ ID NO: 258 - DNA, 20 bp, Artificial sequence
  Synthetic
gcagggtgaaggtcctcctc

SEQ ID NO: 259 - DNA, 20 bp, Artificial sequence
  Synthetic
gaagagactacaattattct

SEQ ID NO: 260 - DNA, 20 bp, Artificial sequence
  Synthetic
gatatcctttgtgttaactt

SEQ ID NO: 261 - DNA, 20 bp, Artificial sequence
  Synthetic
gaatgactcgcatgacttta

SEQ ID NO: 262 - DNA, 20 bp, Artificial sequence
  Synthetic
ggatgttcaaaccttcaaaa

SEQ ID NO: 263 - DNA, 20 bp, Artificial sequence
  Synthetic
gagaatatatgtttccatta

SEQ ID NO: 264 - DNA, 20 bp, Artificial sequence
  Synthetic
ggaaaagtaatgaatcatac

SEQ ID NO: 265 - DNA, 20 bp, Artificial sequence
  Synthetic
gttacacgaagcacagggtg

SEQ ID NO: 266 - DNA, 20 bp, Artificial sequence
  Synthetic
gaactaggtgctcaaggaat

SEQ ID NO: 267 - DNA, 20 bp, Artificial sequence
  Synthetic
ggcaaagaccagtctgatac

SEQ ID NO: 268 - DNA, 20 bp, Artificial sequence
  Synthetic
gtctagtttcacaataattt

SEQ ID NO: 269 - DNA, 20 bp, Artificial sequence
  Synthetic
gctttatataagatatgaga

SEQ ID NO: 270 - DNA, 20 bp, Artificial sequence
  Synthetic
gcataggatattatatttcg

SEQ ID NO: 271 - DNA, 20 bp, Artificial sequence
  Synthetic
gaccttgactgctcctgaac

SEQ ID NO: 272 - DNA, 20 bp, Artificial sequence
  Synthetic
gcagctccctagttcacaga

SEQ ID NO: 273 - DNA, 20 bp, Artificial sequence
  Synthetic
gtctgaccagaggtggagag

SEQ ID NO: 274 - DNA, 20 bp, Artificial sequence
  Synthetic
gaatcacattgtaccacaaa

SEQ ID NO: 275 - DNA, 20 bp, Artificial sequence
  Synthetic
gacaaaattgatacaacagc

SEQ ID NO: 276 - DNA, 20 bp, Artificial sequence
  Synthetic
gaattccaagacttcacatt

SEQ ID NO: 277 - DNA, 20 bp, Artificial sequence
  Synthetic
gacagggaccgccatccact

SEQ ID NO: 278 - DNA, 20 bp, Artificial sequence
  Synthetic
gttgtatggttcctaaggat

SEQ ID NO: 279 - DNA, 20 bp, Artificial sequence
  Synthetic
gaatatccactactagcttt

SEQ ID NO: 280 - DNA, 20 bp, Artificial sequence
  Synthetic
gccattaatcatgatctgga

SEQ ID NO: 281 - DNA, 20 bp, Artificial sequence
  Synthetic
ggtgaataggtaggtattga

SEQ ID NO: 282 - DNA, 20 bp, Artificial sequence
  Synthetic
gctcatcaaaggtagtaaac

SEQ ID NO: 283 - DNA, 20 bp, Artificial sequence
  Synthetic
gggacccagcccttgggctg

SEQ ID NO: 284 - DNA, 20 bp, Artificial sequence
  Synthetic
gtgcacctttctataaatgt

SEQ ID NO: 285 - DNA, 20 bp, Artificial sequence
  Synthetic
gacttcattaaaagcagtct

SEQ ID NO: 286 - DNA, 20 bp, Artificial sequence
  Synthetic
gttgaacttgtgaacacaaa

SEQ ID NO: 287 - DNA, 20 bp, Artificial sequence
  Synthetic
gggtcctcaccaggaaattt

SEQ ID NO: 288 - DNA, 20 bp, Artificial sequence
  Synthetic
gtagcctattggcaattggc

SEQ ID NO: 289 - DNA, 20 bp, Artificial sequence
  Synthetic
gcataaataaaatcgattcc

SEQ ID NO: 290 - DNA, 20 bp, Artificial sequence
  Synthetic
gaagggcaataattggtaca

SEQ ID NO: 291 - DNA, 20 bp, Artificial sequence
  Synthetic
gagttcttaataacattcta

SEQ ID NO: 292 - DNA, 20 bp, Artificial sequence
  Synthetic
gctttctacttgccttagat

SEQ ID NO: 293 - DNA, 20 bp, Artificial sequence
  Synthetic
gcttcttatttctctccagt

SEQ ID NO: 294 - DNA, 20 bp, Artificial sequence
  Synthetic
gcattctgtcctaataagaa

SEQ ID NO: 295 - DNA, 20 bp, Artificial sequence
  Synthetic
gcttaagctagtttaaagaa

SEQ ID NO: 296 - DNA, 20 bp, Artificial sequence
  Synthetic
ggtttccagtgtttatctgt

SEQ ID NO: 297 - DNA, 20 bp, Artificial sequence
  Synthetic
gagagtctaggtacgttctc

SEQ ID NO: 298 - DNA, 20 bp, Artificial sequence
  Synthetic
gctttcaagttaacatagct

SEQ ID NO: 299 - DNA, 20 bp, Artificial sequence
  Synthetic
gtaaaatgaaccgagcttta

SEQ ID NO: 300 - DNA, 20 bp, Artificial sequence
  Synthetic
gtaagattattaaccccttc

SEQ ID NO: 301 - DNA, 20 bp, Artificial sequence
  Synthetic
gggtcctcacgatagaagaa

SEQ ID NO: 302 - DNA, 20 bp, Artificial sequence
  Synthetic
gattacactcaagaaagcga

SEQ ID NO: 303 - DNA, 20 bp, Artificial sequence
  Synthetic
gatgtagacgtagaagtgat

SEQ ID NO: 304 - DNA, 20 bp, Artificial sequence
  Synthetic
gtgagttacagaaattagca

SEQ ID NO: 305 - DNA, 20 bp, Artificial sequence
  Synthetic
gcagggggacacgggcacat

SEQ ID NO: 306 - DNA, 20 bp, Artificial sequence
  Synthetic
gacaattgtgttgcagacaa

SEQ ID NO: 307 - DNA, 20 bp, Artificial sequence
  Synthetic
gtcaatgggaaattataaac

SEQ ID NO: 308 - DNA, 20 bp, Artificial sequence
  Synthetic
gagttatagcacacttagaa

SEQ ID NO: 309 - DNA, 20 bp, Artificial sequence
  Synthetic
gattgaaaccagaaaataag

SEQ ID NO: 310 - DNA, 20 bp, Artificial sequence
  Synthetic
ggagtctagtgataggggta

SEQ ID NO: 311 - DNA, 20 bp, Artificial sequence
  Synthetic
gggatagtcttagaaggctt

SEQ ID NO: 312 - DNA, 20 bp, Artificial sequence
  Synthetic
gtcaattgattcactggaat

SEQ ID NO: 313 - DNA, 20 bp, Artificial sequence
  Synthetic
gtattcctgcaagataattc

SEQ ID NO: 314 - DNA, 20 bp, Artificial sequence
  Synthetic
ggtcaagcaacaggcataat

SEQ ID NO: 315 - DNA, 20 bp, Artificial sequence
  Synthetic
gacatccataacttcctaac

SEQ ID NO: 316 - DNA, 20 bp, Artificial sequence
  Synthetic
gtcaaacaaaagcgtctata

SEQ ID NO: 317 - DNA, 20 bp, Artificial sequence
  Synthetic
gctagattaatatgaatgag

SEQ ID NO: 318 - DNA, 20 bp, Artificial sequence
  Synthetic
gaaccccataggaggtttag

SEQ ID NO: 319 - DNA, 20 bp, Artificial sequence
  Synthetic
gcctctttcccctgccggca

SEQ ID NO: 320 - DNA, 20 bp, Artificial sequence
  Synthetic
ggtaagggctgcttatcttt

SEQ ID NO: 321 - DNA, 20 bp, Artificial sequence
  Synthetic
gtattcagtataatcaagga

SEQ ID NO: 322 - DNA, 20 bp, Artificial sequence
  Synthetic
gttgtcttatgggactgcat

SEQ ID NO: 323 - DNA, 20 bp, Artificial sequence
  Synthetic
gtatacgatatgattgactc

SEQ ID NO: 324 - DNA, 20 bp, Artificial sequence
  Synthetic
ggtagagacaaaatatattt

SEQ ID NO: 325 - DNA, 20 bp, Artificial sequence
  Synthetic
gtacctatgtccttgaggct

SEQ ID NO: 326 - DNA, 20 bp, Artificial sequence
  Synthetic
ggcaaaagaacgtctgtaat

SEQ ID NO: 327 - DNA, 20 bp, Artificial sequence
  Synthetic
ggactagtttacctagggag

SEQ ID NO: 328 - DNA, 20 bp, Artificial sequence
  Synthetic
ggagggtggagcaaagaaag

SEQ ID NO: 329 - DNA, 20 bp, Artificial sequence
  Synthetic
gagccatattatgtccttta

SEQ ID NO: 330 - DNA, 20 bp, Artificial sequence
  Synthetic
gtgcactctatgcaccaaag

SEQ ID NO: 331 - DNA, 20 bp, Artificial sequence
  Synthetic
ggtctcccgagtcattgttg

SEQ ID NO: 332 - DNA, 20 bp, Artificial sequence
  Synthetic
gcaatcattctggttcaggc

SEQ ID NO: 333 - DNA, 20 bp, Artificial sequence
  Synthetic
gcacaggttcccctcctaac

SEQ ID NO: 334 - DNA, 20 bp, Artificial sequence
  Synthetic
gatcagggaatctttgagaa

SEQ ID NO: 335 - DNA, 20 bp, Artificial sequence
  Synthetic
gaacccagctgtcctcgctg

SEQ ID NO: 336 - DNA, 20 bp, Artificial sequence
  Synthetic
gctaactgtgttacaagcag

SEQ ID NO: 337 - DNA, 20 bp, Artificial sequence
  Synthetic
gtgatcaaagagagaggtgt

SEQ ID NO: 338 - DNA, 20 bp, Artificial sequence
  Synthetic
ggaaagcccgttgtatttat

SEQ ID NO: 339 - DNA, 20 bp, Artificial sequence
  Synthetic
ggtcccccactttctccttg

SEQ ID NO: 340 - DNA, 20 bp, Artificial sequence
  Synthetic
gccagatgaccatagaaact

SEQ ID NO: 341 - DNA, 20 bp, Artificial sequence
  Synthetic
ggtgcaatccaaaggtgggc

SEQ ID NO: 342 - DNA, 20 bp, Artificial sequence
  Synthetic
gtgtaaaatcactttaaact

SEQ ID NO: 343 - DNA, 20 bp, Artificial sequence
  Synthetic
gtcacatgttcaagtttaac

SEQ ID NO: 344 - DNA, 20 bp, Artificial sequence
  Synthetic
gaagcttagtcctgaattgt

SEQ ID NO: 345 - DNA, 20 bp, Artificial sequence
  Synthetic
gggtctgtttccttgtgtta

SEQ ID NO: 346 - DNA, 20 bp, Artificial sequence
  Synthetic
gatagagactggatgaagtt

SEQ ID NO: 347 - DNA, 20 bp, Artificial sequence
  Synthetic
gcaacaaggcaaatgtggta

SEQ ID NO: 348 - DNA, 20 bp, Artificial sequence
  Synthetic
gctatttagctcaaccttgt

SEQ ID NO: 349 - DNA, 20 bp, Artificial sequence
  Synthetic
gtgccattatcatttcctca

SEQ ID NO: 350 - DNA, 20 bp, Artificial sequence
  Synthetic
gcaaatagaagagacaatct

SEQ ID NO: 351 - DNA, 20 bp, Artificial sequence
  Synthetic
gaaaatatatggactgggat

SEQ ID NO: 352 - DNA, 20 bp, Artificial sequence
  Synthetic
gaatagaactcctgccatca

SEQ ID NO: 353 - DNA, 20 bp, Artificial sequence
  Synthetic
gctttctacctggatgttta

SEQ ID NO: 354 - DNA, 20 bp, Artificial sequence
  Synthetic
gctaacttgagggcaaaaga

SEQ ID NO: 355 - DNA, 20 bp, Artificial sequence
  Synthetic
gtggtaaaaatgtgctttgt

SEQ ID NO: 356 - DNA, 20 bp, Artificial sequence
  Synthetic
gagcctcagctggtgcatgg

SEQ ID NO: 357 - DNA, 20 bp, Artificial sequence
  Synthetic
gcctatgccgcaataccctc

SEQ ID NO: 358 - DNA, 20 bp, Artificial sequence
  Synthetic
gacctgtgtaaaccagctaa

SEQ ID NO: 359 - DNA, 20 bp, Artificial sequence
  Synthetic
gacctcattcctgagtgtgt

SEQ ID NO: 360 - DNA, 20 bp, Artificial sequence
  Synthetic
gtgtttgcctcataataacc

SEQ ID NO: 361 - DNA, 20 bp, Artificial sequence
  Synthetic
gactgggcatacagccattt

SEQ ID NO: 362 - DNA, 20 bp, Artificial sequence
  Synthetic
ggcatactacattggcttta

SEQ ID NO: 363 - DNA, 20 bp, Artificial sequence
  Synthetic
gcaaacatattggagtactg

SEQ ID NO: 364 - DNA, 20 bp, Artificial sequence
  Synthetic
ggggagtagggaagagctta

SEQ ID NO: 365 - DNA, 20 bp, Artificial sequence
  Synthetic
gggctcgtatgtcgttcttc

SEQ ID NO: 366 - DNA, 20 bp, Artificial sequence
  Synthetic
gtgccttatctatttccaca

SEQ ID NO: 367 - DNA, 20 bp, Artificial sequence
  Synthetic
ggtaattacctgctctctgc

SEQ ID NO: 368 - DNA, 20 bp, Artificial sequence
  Synthetic
gtctgataacttgtgttact

SEQ ID NO: 369 - DNA, 20 bp, Artificial sequence
  Synthetic
gactgagttaataatagcgg

SEQ ID NO: 370 - DNA, 20 bp, Artificial sequence
  Synthetic
gaatattgtgcactgtattt

SEQ ID NO: 371 - DNA, 20 bp, Artificial sequence
  Synthetic
gtttctaaatgtgatctgtg

SEQ ID NO: 372 - DNA, 20 bp, Artificial sequence
  Synthetic
gcacactggctagttaagga

SEQ ID NO: 373 - DNA, 20 bp, Artificial sequence
  Synthetic
ggaggagtgtgcaatgaagc

SEQ ID NO: 374 - DNA, 20 bp, Artificial sequence
  Synthetic
gaggacgggtgggaagttag

SEQ ID NO: 375 - DNA, 20 bp, Artificial sequence
  Synthetic
gatactgtagcagttactga

SEQ ID NO: 376 - DNA, 20 bp, Artificial sequence
  Synthetic
gattctaagcaaaggacaga

SEQ ID NO: 377 - DNA, 20 bp, Artificial sequence
  Synthetic
ggagcttagaccatatttgg

SEQ ID NO: 378 - DNA, 20 bp, Artificial sequence
  Synthetic
gtgtccgtgggtctgttccc

SEQ ID NO: 379 - DNA, 20 bp, Artificial sequence
  Synthetic
gcaatagctgtgagctcata

SEQ ID NO: 380 - DNA, 20 bp, Artificial sequence
  Synthetic
gggatgggccatccagctgt

SEQ ID NO: 381 - DNA, 20 bp, Artificial sequence
  Synthetic
gacagattacttaataaaag

SEQ ID NO: 382 - DNA, 20 bp, Artificial sequence
  Synthetic
gtggcaaggttaagtacaat

SEQ ID NO: 383 - DNA, 20 bp, Artificial sequence
  Synthetic
ggaggaaacagaataatggc

SEQ ID NO: 384 - DNA, 20 bp, Artificial sequence
  Synthetic
gtgaattaatgtcatttcac

SEQ ID NO: 385 - DNA, 20 bp, Artificial sequence
  Synthetic
gtgaactagaacactgagag

SEQ ID NO: 386 - DNA, 20 bp, Artificial sequence
  Synthetic
gatgctgtggccaatgtgca

SEQ ID NO: 387 - DNA, 20 bp, Artificial sequence
  Synthetic
gactgtaagcattcctgaca

SEQ ID NO: 388 - DNA, 20 bp, Artificial sequence
  Synthetic
gtcctaattccatgcctaaa

SEQ ID NO: 389 - DNA, 20 bp, Artificial sequence
  Synthetic
gtgggttcgttgtctactac

SEQ ID NO: 390 - DNA, 20 bp, Artificial sequence
  Synthetic
gagactattagatcgtatgt

SEQ ID NO: 391 - DNA, 20 bp, Artificial sequence
  Synthetic
ggtgtagtatcaaaaattga

SEQ ID NO: 392 - DNA, 20 bp, Artificial sequence
  Synthetic
gatagctcttaaggataaat

SEQ ID NO: 393 - DNA, 20 bp, Artificial sequence
  Synthetic
gattcagtcacatcacaata

SEQ ID NO: 394 - DNA, 20 bp, Artificial sequence
  Synthetic
gtctaagaaagacttctagg

SEQ ID NO: 395 - DNA, 20 bp, Artificial sequence
  Synthetic
gatttgggtctttgcgcatc

SEQ ID NO: 396 - DNA, 20 bp, Artificial sequence
  Synthetic
gaccttaaagttatagttaa

SEQ ID NO: 397 - DNA, 20 bp, Artificial sequence
  Synthetic
gctctgcatctttccccagg

SEQ ID NO: 398 - DNA, 20 bp, Artificial sequence
  Synthetic
gacctaagtttgagaatgag

SEQ ID NO: 399 - DNA, 20 bp, Artificial sequence
  Synthetic
gaaagtacattcattagcat

SEQ ID NO: 400 - DNA, 20 bp, Artificial sequence
  Synthetic
ggagaacgtggtgataaagc

SEQ ID NO: 401 - DNA, 20 bp, Artificial sequence
  Synthetic
ggcaacatggcaaaatagtt

SEQ ID NO: 402 - DNA, 20 bp, Artificial sequence
  Synthetic
gataatagcagagagaggtg

SEQ ID NO: 403 - DNA, 20 bp, Artificial sequence
  Synthetic
ggactttaaggaattcagct

SEQ ID NO: 404 - DNA, 20 bp, Artificial sequence
  Synthetic
gaatattggggggtggatgg

SEQ ID NO: 405 - DNA, 20 bp, Artificial sequence
  Synthetic
ggagtaagtatgtgtgttga

SEQ ID NO: 406 - DNA, 20 bp, Artificial sequence
  Synthetic
gtattggataagggagctca

SEQ ID NO: 407 - DNA, 20 bp, Artificial sequence
  Synthetic
gtgagttgggagatgtactg

SEQ ID NO: 408 - DNA, 20 bp, Artificial sequence
  Synthetic
gtttacaatttcatttgtac

SEQ ID NO: 409 - DNA, 20 bp, Artificial sequence
  Synthetic
gtccattcaatttggacatg

SEQ ID NO: 410 - DNA, 20 bp, Artificial sequence
  Synthetic
gagtgcttactgggaatgag

SEQ ID NO: 411 - DNA, 20 bp, Artificial sequence
  Synthetic
gctaattgttcaaaaagccc

SEQ ID NO: 412 - DNA, 20 bp, Artificial sequence
  Synthetic
gctttcaagagtttatttga

SEQ ID NO: 413 - DNA, 20 bp, Artificial sequence
  Synthetic
gatattctgtgcaatctgtt

SEQ ID NO: 414 - DNA, 20 bp, Artificial sequence
  Synthetic
gtgtaggactacgctggcac

SEQ ID NO: 415 - DNA, 20 bp, Artificial sequence
  Synthetic
gtcttaaagagtaaagtaca

SEQ ID NO: 416 - DNA, 20 bp, Artificial sequence
  Synthetic
gttagactgcaaacacccac

SEQ ID NO: 417 - DNA, 20 bp, Artificial sequence
  Synthetic
gcctaggagaagccctggca

SEQ ID NO: 418 - DNA, 20 bp, Artificial sequence
  Synthetic
gtcgagtatttctaatcttt

SEQ ID NO: 419 - DNA, 20 bp, Artificial sequence
  Synthetic
gaatctgagacatcattcat

SEQ ID NO: 420 - DNA, 20 bp, Artificial sequence
  Synthetic
gacaaaagattatgcttccc

SEQ ID NO: 421 - DNA, 20 bp, Artificial sequence
  Synthetic
gagaattacattcatgatct

SEQ ID NO: 422 - DNA, 20 bp, Artificial sequence
  Synthetic
gaactgagcttctaccatgc

SEQ ID NO: 423 - DNA, 20 bp, Artificial sequence
  Synthetic
ggtaagattgtaatagcttg

SEQ ID NO: 424 - DNA, 20 bp, Artificial sequence
  Synthetic
gtcagaaatgatctcgtcct

SEQ ID NO: 425 - DNA, 20 bp, Artificial sequence
  Synthetic
gacatatctaagaactgagc

SEQ ID NO: 426 - DNA, 20 bp, Artificial sequence
  Synthetic
gcttcaatatgacagaactc

SEQ ID NO: 427 - DNA, 20 bp, Artificial sequence
  Synthetic
ggagagcaaatcagcatatc

SEQ ID NO: 428 - DNA, 20 bp, Artificial sequence
  Synthetic
gcaaaatagccgcacagaaa

SEQ ID NO: 429 - DNA, 20 bp, Artificial sequence
  Synthetic
gcatatttctatacaataca

SEQ ID NO: 430 - DNA, 20 bp, Artificial sequence
  Synthetic
gatgcaaattcatggtggta

SEQ ID NO: 431 - DNA, 20 bp, Artificial sequence
  Synthetic
gaactgtaatagtcttgagc

SEQ ID NO: 432 - DNA, 20 bp, Artificial sequence
  Synthetic
gaactcactacattaaggct

SEQ ID NO: 433 - DNA, 20 bp, Artificial sequence
  Synthetic
gaggtaaatcagtacaaaca

SEQ ID NO: 434 - DNA, 20 bp, Artificial sequence
  Synthetic
gttgtttctaagattaaaag

SEQ ID NO: 435 - DNA, 20 bp, Artificial sequence
  Synthetic
gtggtagtcagtttcacaaa

SEQ ID NO: 436 - DNA, 20 bp, Artificial sequence
  Synthetic
ggtttcaaatagttggatca

SEQ ID NO: 437 - DNA, 20 bp, Artificial sequence
  Synthetic
gaatatgaaagacatcataa

SEQ ID NO: 438 - DNA, 20 bp, Artificial sequence
  Synthetic
gaagtaggaaggagattgcc

SEQ ID NO: 439 - DNA, 20 bp, Artificial sequence
  Synthetic
ggaaaagtgctgtttgcatt

SEQ ID NO: 440 - DNA, 20 bp, Artificial sequence
  Synthetic
gagcattaggctggggcctt

SEQ ID NO: 441 - DNA, 20 bp, Artificial sequence
  Synthetic
gtctaggtatgattagaaga

SEQ ID NO: 442 - DNA, 20 bp, Artificial sequence
  Synthetic
gagttataatcttcagaaaa

SEQ ID NO: 443 - DNA, 20 bp, Artificial sequence
  Synthetic
gctgtaatgagacttcagct

SEQ ID NO: 444 - DNA, 20 bp, Artificial sequence
  Synthetic
gtgtgcaatctgaaggaaat

SEQ ID NO: 445 - DNA, 20 bp, Artificial sequence
  Synthetic
gtgatgaggtcgctgaagtt

SEQ ID NO: 446 - DNA, 20 bp, Artificial sequence
  Synthetic
gtggagcccttataaccctg

SEQ ID NO: 447 - DNA, 20 bp, Artificial sequence
  Synthetic
gttggattatttcttctata

SEQ ID NO: 448 - DNA, 20 bp, Artificial sequence
  Synthetic
ggatttctacattatatact

SEQ ID NO: 449 - DNA, 20 bp, Artificial sequence
  Synthetic
gctaatgtagatcaagttat

SEQ ID NO: 450 - DNA, 20 bp, Artificial sequence
  Synthetic
gattgcaagagactgaactc

SEQ ID NO: 451 - DNA, 20 bp, Artificial sequence
  Synthetic
gggtgaacttgagtgaactt

SEQ ID NO: 452 - DNA, 20 bp, Artificial sequence
  Synthetic
gggctcaaatccctataatt

SEQ ID NO: 453 - DNA, 20 bp, Artificial sequence
  Synthetic
gatagaaggtattaactccc

SEQ ID NO: 454 - DNA, 20 bp, Artificial sequence
  Synthetic
ggctataagcacaaatgtaa

SEQ ID NO: 455 - DNA, 20 bp, Artificial sequence
  Synthetic
gattcccattgcatgccagt

SEQ ID NO: 456 - DNA, 20 bp, Artificial sequence
  Synthetic
gcaaattacaattatgtttc

SEQ ID NO: 457 - DNA, 20 bp, Artificial sequence
  Synthetic
gaattaaattcactttgaac

SEQ ID NO: 458 - DNA, 20 bp, Artificial sequence
  Synthetic
gagcagacaggaaataaagc

SEQ ID NO: 459 - DNA, 20 bp, Artificial sequence
  Synthetic
gcccaccagtccttctcact

SEQ ID NO: 460 - DNA, 20 bp, Artificial sequence
  Synthetic
gttaagaagtgaaagaaatt

SEQ ID NO: 461 - DNA, 20 bp, Artificial sequence
  Synthetic
gttgaattgaatgggtcatt

SEQ ID NO: 462 - DNA, 20 bp, Artificial sequence
  Synthetic
gtagacacaaacttgtgtaa

SEQ ID NO: 463 - DNA, 20 bp, Artificial sequence
  Synthetic
gagcgtactatattcttaaa

SEQ ID NO: 464 - DNA, 20 bp, Artificial sequence
  Synthetic
ggtggtacatcgttgaagga

SEQ ID NO: 465 - DNA, 20 bp, Artificial sequence
  Synthetic
gatgaactcccaatcacagg

SEQ ID NO: 466 - DNA, 20 bp, Artificial sequence
  Synthetic
gtataaataaggataaggta

SEQ ID NO: 467 - DNA, 20 bp, Artificial sequence
  Synthetic
ggaaataatcttggaacata

SEQ ID NO: 468 - DNA, 20 bp, Artificial sequence
  Synthetic
ggtagttaatcttctacttt

SEQ ID NO: 469 - DNA, 20 bp, Artificial sequence
  Synthetic
gagaagagaacattctagtt

SEQ ID NO: 470 - DNA, 20 bp, Artificial sequence
  Synthetic
gtcggagctcagtgttgcat

SEQ ID NO: 471 - DNA, 20 bp, Artificial sequence
  Synthetic
gaagagacatgtttcagtga

SEQ ID NO: 472 - DNA, 20 bp, Artificial sequence
  Synthetic
gtcatatctgacttaaattg

SEQ ID NO: 473 - DNA, 20 bp, Artificial sequence
  Synthetic
ggagaatatgctaaaagcgt

SEQ ID NO: 474 - DNA, 20 bp, Artificial sequence
  Synthetic
gattgttgtagtagaataaa

SEQ ID NO: 475 - DNA, 20 bp, Artificial sequence
  Synthetic
gtaagcagcaccaccactta

SEQ ID NO: 476 - DNA, 20 bp, Artificial sequence
  Synthetic
gtcttgtgctgacatgctca

SEQ ID NO: 477 - DNA, 20 bp, Artificial sequence
  Synthetic
gcagactttattagctagtg

SEQ ID NO: 478 - DNA, 20 bp, Artificial sequence
  Synthetic
gaggtatttgatatgactca

SEQ ID NO: 479 - DNA, 20 bp, Artificial sequence
  Synthetic
gcaggttgcccattctccca

SEQ ID NO: 480 - DNA, 20 bp, Artificial sequence
  Synthetic
gaggggacgttgacctgtgg

SEQ ID NO: 481 - DNA, 20 bp, Artificial sequence
  Synthetic
gaacccaaggatttataaag

SEQ ID NO: 482 - DNA, 20 bp, Artificial sequence
  Synthetic
gtgttcaggacatgtactca

SEQ ID NO: 483 - DNA, 20 bp, Artificial sequence
  Synthetic
ggtgatgatagtcaaatacc

SEQ ID NO: 484 - DNA, 20 bp, Artificial sequence
  Synthetic
gctttacagctaatttctaa

SEQ ID NO: 485 - DNA, 20 bp, Artificial sequence
  Synthetic
ggtatctacattaacactca

SEQ ID NO: 486 - DNA, 20 bp, Artificial sequence
  Synthetic
gacagtttgcttactatgga

SEQ ID NO: 487 - DNA, 20 bp, Artificial sequence
  Synthetic
gaaaaactcttagcttaatg

SEQ ID NO: 488 - DNA, 20 bp, Artificial sequence
  Synthetic
gtcatcttaacttcagtaga

SEQ ID NO: 489 - DNA, 20 bp, Artificial sequence
  Synthetic
gatcactggtaggccacagt

SEQ ID NO: 490 - DNA, 20 bp, Artificial sequence
  Synthetic
gagaaaggcaagtgcatcaa

SEQ ID NO: 491 - DNA, 20 bp, Artificial sequence
  Synthetic
gaactgataaagattcagta

SEQ ID NO: 492 - DNA, 20 bp, Artificial sequence
  Synthetic
gccattcaaaagcagctata

SEQ ID NO: 493 - DNA, 20 bp, Artificial sequence
  Synthetic
gacagaacttctttgagcta

SEQ ID NO: 494 - DNA, 20 bp, Artificial sequence
  Synthetic
gggtgacattgaaatttaac

SEQ ID NO: 495 - DNA, 20 bp, Artificial sequence
  Synthetic
gactataaactgcacactat

SEQ ID NO: 496 - DNA, 20 bp, Artificial sequence
  Synthetic
gctatggtgggaaagctcat

SEQ ID NO: 497 - DNA, 20 bp, Artificial sequence
  Synthetic
gactaacttgctaatggcta

SEQ ID NO: 498 - DNA, 20 bp, Artificial sequence
  Synthetic
gagagtcacttcaaagtgtg

SEQ ID NO: 499 - DNA, 20 bp, Artificial sequence
  Synthetic
gagtgtatttgtggacaata

SEQ ID NO: 500 - DNA, 20 bp, Artificial sequence
  Synthetic
gaagaattagggttccattt

SEQ ID NO: 501 - DNA, 20 bp, Artificial sequence
  Synthetic
gaggagtggcactttatact

SEQ ID NO: 502 - DNA, 20 bp, Artificial sequence
  Synthetic
gaaggatgcagtagccattg

SEQ ID NO: 503 - DNA, 20 bp, Artificial sequence
  Synthetic
gtgcattgttggtggttgtg

SEQ ID NO: 504 - DNA, 20 bp, Artificial sequence
  Synthetic
gagaagttatgcaaatttat

SEQ ID NO: 505 - DNA, 20 bp, Artificial sequence
  Synthetic
gaaatagattggcagagtgt

SEQ ID NO: 506 - DNA, 20 bp, Artificial sequence
  Synthetic
gtggggtgggctccctgcct

SEQ ID NO: 507 - DNA, 20 bp, Artificial sequence
  Synthetic
gtctctaacaagactgaaat

SEQ ID NO: 508 - DNA, 20 bp, Artificial sequence
  Synthetic
gcagagtagatctacatctt

SEQ ID NO: 509 - DNA, 20 bp, Artificial sequence
  Synthetic
gtgccagctaagatgaaatt

SEQ ID NO: 510 - DNA, 20 bp, Artificial sequence
  Synthetic
gatggtgatgcaccaacttt

SEQ ID NO: 511 - DNA, 20 bp, Artificial sequence
  Synthetic
gaagtgttgccattcaattc

SEQ ID NO: 512 - DNA, 20 bp, Artificial sequence
  Synthetic
gagagagttggaataagcta

SEQ ID NO: 513 - DNA, 20 bp, Artificial sequence
  Synthetic
gagggtacttatttcaactt

SEQ ID NO: 514 - DNA, 20 bp, Artificial sequence
  Synthetic
gctacatgttctagaataca

SEQ ID NO: 515 - DNA, 20 bp, Artificial sequence
  Synthetic
gagaaatctctttgagctgg

SEQ ID NO: 516 - DNA, 20 bp, Artificial sequence
  Synthetic
ggctttgtgtctgactttcc

SEQ ID NO: 517 - DNA, 20 bp, Artificial sequence
  Synthetic
ggattagatcaattattcta

SEQ ID NO: 518 - DNA, 20 bp, Artificial sequence
  Synthetic
gattctggaaataagtacct

SEQ ID NO: 519 - DNA, 20 bp, Artificial sequence
  Synthetic
gagataaaattgcgagacca

SEQ ID NO: 520 - DNA, 20 bp, Artificial sequence
  Synthetic
gacaaaatttagcaactcag

SEQ ID NO: 521 - DNA, 20 bp, Artificial sequence
  Synthetic
gcagatactcaccattaccc

SEQ ID NO: 522 - DNA, 20 bp, Artificial sequence
  Synthetic
ggtgattgttgcagctgtca

SEQ ID NO: 523 - DNA, 20 bp, Artificial sequence
  Synthetic
gatagacttgtgaaggaaac

SEQ ID NO: 524 - DNA, 20 bp, Artificial sequence
  Synthetic
gagtcactggattgttgtcc

SEQ ID NO: 525 - DNA, 20 bp, Artificial sequence
  Synthetic
ggattatatgggaggtacac

SEQ ID NO: 526 - DNA, 20 bp, Artificial sequence
  Synthetic
gcttaaaaatactatctgct

SEQ ID NO: 527 - DNA, 20 bp, Artificial sequence
  Synthetic
gacaaggaggaccaaagttg

SEQ ID NO: 528 - DNA, 20 bp, Artificial sequence
  Synthetic
ggcagtgatttactcctatc

SEQ ID NO: 529 - DNA, 20 bp, Artificial sequence
  Synthetic
gatcttccaggactgttaga

SEQ ID NO: 530 - DNA, 20 bp, Artificial sequence
  Synthetic
gaaacaagctaatattatca

SEQ ID NO: 531 - DNA, 20 bp, Artificial sequence
  Synthetic
gtcagtctttacaaatcact

SEQ ID NO: 532 - DNA, 20 bp, Artificial sequence
  Synthetic
ggcagttgagtaaacgtaag

SEQ ID NO: 533 - DNA, 20 bp, Artificial sequence
  Synthetic
gcctctactgctaactctat

SEQ ID NO: 534 - DNA, 20 bp, Artificial sequence
  Synthetic
gttgtaatttaaagcactca

SEQ ID NO: 535 - DNA, 20 bp, Artificial sequence
  Synthetic
gcataaagagaacaagcaat

SEQ ID NO: 536 - DNA, 20 bp, Artificial sequence
  Synthetic
ggtagttggtctaatcagta

SEQ ID NO: 537 - DNA, 20 bp, Artificial sequence
  Synthetic
ggctaacacctgccaacttt

SEQ ID NO: 538 - DNA, 20 bp, Artificial sequence
  Synthetic
gtctaatctagcatcaaact

SEQ ID NO: 539 - DNA, 20 bp, Artificial sequence
  Synthetic
gagagagactatttcaggat

SEQ ID NO: 540 - DNA, 20 bp, Artificial sequence
  Synthetic
gacctagaccaagctacgaa

SEQ ID NO: 541 - DNA, 20 bp, Artificial sequence
  Synthetic
gttactgataccagtccctg

SEQ ID NO: 542 - DNA, 20 bp, Artificial sequence
  Synthetic
gccctactgtggtaactttg

SEQ ID NO: 543 - DNA, 20 bp, Artificial sequence
  Synthetic
gtgtaaaggaatcttagctt

SEQ ID NO: 544 - DNA, 20 bp, Artificial sequence
  Synthetic
ggtgagactattatatttat

SEQ ID NO: 545 - DNA, 20 bp, Artificial sequence
  Synthetic
gcttcagagaactatttggt

SEQ ID NO: 546 - DNA, 20 bp, Artificial sequence
  Synthetic
gatgtgttcgttgaggcata

SEQ ID NO: 547 - DNA, 20 bp, Artificial sequence
  Synthetic
gttgactctaactatagagt

SEQ ID NO: 548 - DNA, 20 bp, Artificial sequence
  Synthetic
ggacagccattgaagatatg

SEQ ID NO: 549 - DNA, 20 bp, Artificial sequence
  Synthetic
gatggagagcctggagcata

SEQ ID NO: 550 - DNA, 20 bp, Artificial sequence
  Synthetic
gcatgattaaaggtgagcat

SEQ ID NO: 551 - DNA, 20 bp, Artificial sequence
  Synthetic
ggaacccacagatatagcta

SEQ ID NO: 552 - DNA, 20 bp, Artificial sequence
  Synthetic
gcatagcttcagagttcaga

SEQ ID NO: 553 - DNA, 20 bp, Artificial sequence
  Synthetic
gagaaaagacgtgtatttcc

SEQ ID NO: 554 - DNA, 20 bp, Artificial sequence
  Synthetic
gctagagcttccttatgttt

SEQ ID NO: 555 - DNA, 20 bp, Artificial sequence
  Synthetic
gatgggcagtcaggactacg

SEQ ID NO: 556 - DNA, 20 bp, Artificial sequence
  Synthetic
gttctgcatgagaagcacta

SEQ ID NO: 557 - DNA, 20 bp, Artificial sequence
  Synthetic
gactccacctatctcaaaat

SEQ ID NO: 558 - DNA, 20 bp, Artificial sequence
  Synthetic
gatatttgacagtggataaa

SEQ ID NO: 559 - DNA, 20 bp, Artificial sequence
  Synthetic
gaaagattatggatcatagt

SEQ ID NO: 560 - DNA, 20 bp, Artificial sequence
  Synthetic
gcatcaatgtacactgtggc

SEQ ID NO: 561 - DNA, 20 bp, Artificial sequence
  Synthetic
gcagcaagctatggtccatg

SEQ ID NO: 562 - DNA, 20 bp, Artificial sequence
  Synthetic
ggttgtttgaattaaagact

SEQ ID NO: 563 - DNA, 20 bp, Artificial sequence
  Synthetic
gaacccctggctagtttccc

SEQ ID NO: 564 - DNA, 20 bp, Artificial sequence
  Synthetic
ggataaagagtgaacctgta

SEQ ID NO: 565 - DNA, 20 bp, Artificial sequence
  Synthetic
gtagatttcactaaattgtt

SEQ ID NO: 566 - DNA, 20 bp, Artificial sequence
  Synthetic
gtgtagttagaataagaagg

SEQ ID NO: 567 - DNA, 20 bp, Artificial sequence
  Synthetic
gtggcaatgtcctggagaaa

SEQ ID NO: 568 - DNA, 20 bp, Artificial sequence
  Synthetic
gtgaagtgctttatctgtac

SEQ ID NO: 569 - DNA, 20 bp, Artificial sequence
  Synthetic
gagtttatataggtatgaaa

SEQ ID NO: 570 - DNA, 20 bp, Artificial sequence
  Synthetic
gacctcataaacaaatcact

SEQ ID NO: 571 - DNA, 20 bp, Artificial sequence
  Synthetic
gaaacgtctgtatgcaaagc

SEQ ID NO: 572 - DNA, 20 bp, Artificial sequence
  Synthetic
ggtgtggtgcaagggtgagt

SEQ ID NO: 573 - DNA, 20 bp, Artificial sequence
  Synthetic
gagaatctgctattgccaat

SEQ ID NO: 574 - DNA, 20 bp, Artificial sequence
  Synthetic
gtactaagtatcttgaaatg

SEQ ID NO: 575 - DNA, 20 bp, Artificial sequence
  Synthetic
gtcatgacatgagttgcatg

SEQ ID NO: 576 - DNA, 20 bp, Artificial sequence
  Synthetic
gcagtgatcagagacagttg

SEQ ID NO: 577 - DNA, 20 bp, Artificial sequence
  Synthetic
ggcaaaataacttcatctat

SEQ ID NO: 578 - DNA, 20 bp, Artificial sequence
  Synthetic
gcctggccttctgtggaatt

SEQ ID NO: 579 - DNA, 20 bp, Artificial sequence
  Synthetic
ggtggcctttgtttgcaggc

SEQ ID NO: 580 - DNA, 20 bp, Artificial sequence
  Synthetic
gagatggtatatttgtcaga

SEQ ID NO: 581 - DNA, 20 bp, Artificial sequence
  Synthetic
gggacacccagcatctcaac

SEQ ID NO: 582 - DNA, 20 bp, Artificial sequence
  Synthetic
gtatatgacagtagggttgg

SEQ ID NO: 583 - DNA, 20 bp, Artificial sequence
  Synthetic
ggaccccagaactgaaatca

SEQ ID NO: 584 - DNA, 20 bp, Artificial sequence
  Synthetic
gggcaccactgagaatgtat

SEQ ID NO: 585 - DNA, 20 bp, Artificial sequence
  Synthetic
gggactacaaatatgaaaaa

SEQ ID NO: 586 - DNA, 20 bp, Artificial sequence
  Synthetic
gtaaaattatgagctccagt

SEQ ID NO: 587 - DNA, 20 bp, Artificial sequence
  Synthetic
gattgtgagtgatgagaatc

SEQ ID NO: 588 - DNA, 20 bp, Artificial sequence
  Synthetic
gagactgagggttgctctta

SEQ ID NO: 589 - DNA, 20 bp, Artificial sequence
  Synthetic
gcatagagtgaacactttgg

SEQ ID NO: 590 - DNA, 20 bp, Artificial sequence
  Synthetic
gaagttctcctttaaccaat

SEQ ID NO: 591 - DNA, 20 bp, Artificial sequence
  Synthetic
gaccttgaccaaagatatta

SEQ ID NO: 592 - DNA, 20 bp, Artificial sequence
  Synthetic
gtgtgggcaagagacagtcc

SEQ ID NO: 593 - DNA, 20 bp, Artificial sequence
  Synthetic
gttgggggctctcttgccac

SEQ ID NO: 594 - DNA, 20 bp, Artificial sequence
  Synthetic
ggataaaactctaacagaac

SEQ ID NO: 595 - DNA, 20 bp, Artificial sequence
  Synthetic
ggaaacatattacccctcca

SEQ ID NO: 596 - DNA, 20 bp, Artificial sequence
  Synthetic
gcactattactccactgaga

SEQ ID NO: 597 - DNA, 20 bp, Artificial sequence
  Synthetic
gtgagcagagatcaccttag

SEQ ID NO: 598 - DNA, 20 bp, Artificial sequence
  Synthetic
gggttcatataggtcggaat

SEQ ID NO: 599 - DNA, 20 bp, Artificial sequence
  Synthetic
gtgcccccgattcttccatg

SEQ ID NO: 600 - DNA, 20 bp, Artificial sequence
  Synthetic
ggaacaaaatttgcacataa

SEQ ID NO: 601 - DNA, 20 bp, Artificial sequence
  Synthetic
gagaaagtccaagggtaaaa

SEQ ID NO: 602 - DNA, 20 bp, Artificial sequence
  Synthetic
gcaattaactctacaaggaa

SEQ ID NO: 603 - DNA, 20 bp, Artificial sequence
  Synthetic
gtttcaaccattagggggct

SEQ ID NO: 604 - DNA, 20 bp, Artificial sequence
  Synthetic
ggcaggggtagtaagcttag

SEQ ID NO: 605 - DNA, 20 bp, Artificial sequence
  Synthetic
gtacacatcttcccaatcag

SEQ ID NO: 606 - DNA, 20 bp, Artificial sequence
  Synthetic
gttacttggaaaaatgacca

SEQ ID NO: 607 - DNA, 20 bp, Artificial sequence
  Synthetic
gtacccggtaaatcatagag

SEQ ID NO: 608 - DNA, 20 bp, Artificial sequence
  Synthetic
gtgtattatcctgcattcca

SEQ ID NO: 609 - DNA, 20 bp, Artificial sequence
  Synthetic
gggtaaaacaaatgcatcat

SEQ ID NO: 610 - DNA, 20 bp, Artificial sequence
  Synthetic
gtgtgttggcctagggatga

SEQ ID NO: 611 - DNA, 20 bp, Artificial sequence
  Synthetic
ggtgtgataaaacctcagag

SEQ ID NO: 612 - DNA, 20 bp, Artificial sequence
  Synthetic
gagctaattggtcagattct

SEQ ID NO: 613 - DNA, 20 bp, Artificial sequence
  Synthetic
gtaccagagtacagtgtccg

SEQ ID NO: 614 - DNA, 20 bp, Artificial sequence
  Synthetic
ggtcagtgctctatcattta

SEQ ID NO: 615 - DNA, 20 bp, Artificial sequence
  Synthetic
gttgcctatcttcagagtac

SEQ ID NO: 616 - DNA, 20 bp, Artificial sequence
  Synthetic
gaagatgcatggacctacca

SEQ ID NO: 617 - DNA, 20 bp, Artificial sequence
  Synthetic
gaatagacactggttctctg

SEQ ID NO: 618 - DNA, 20 bp, Artificial sequence
  Synthetic
gtcagctcttaacatctggt

SEQ ID NO: 619 - DNA, 20 bp, Artificial sequence
  Synthetic
gataacaaggctcagaaggc

SEQ ID NO: 620 - DNA, 20 bp, Artificial sequence
  Synthetic
gtcaaaacacagtgagctgt

SEQ ID NO: 621 - DNA, 20 bp, Artificial sequence
  Synthetic
gagaatatagctgaaggtgg

SEQ ID NO: 622 - DNA, 20 bp, Artificial sequence
  Synthetic
gggattgaccatcaatacag

SEQ ID NO: 623 - DNA, 20 bp, Artificial sequence
  Synthetic
gaaacccccatctcagtctt

SEQ ID NO: 624 - DNA, 20 bp, Artificial sequence
  Synthetic
gtacagataccactatttgg

SEQ ID NO: 625 - DNA, 20 bp, Artificial sequence
  Synthetic
gagtagctagaggcactctt

SEQ ID NO: 626 - DNA, 20 bp, Artificial sequence
  Synthetic
gagatttgcagtgcatgaat

SEQ ID NO: 627 - DNA, 20 bp, Artificial sequence
  Synthetic
gttcaactaaaggtcttatg

SEQ ID NO: 628 - DNA, 20 bp, Artificial sequence
  Synthetic
gtgtttcactgttctcttca

SEQ ID NO: 629 - DNA, 20 bp, Artificial sequence
  Synthetic
gtgaagtagagattatgtaa

SEQ ID NO: 630 - DNA, 20 bp, Artificial sequence
  Synthetic
gtcaaaccaagttgaattca

SEQ ID NO: 631 - DNA, 20 bp, Artificial sequence
  Synthetic
gatgctaaaaatctaaacct

SEQ ID NO: 632 - DNA, 20 bp, Artificial sequence
  Synthetic
ggcccttattaccagatttg

SEQ ID NO: 633 - DNA, 20 bp, Artificial sequence
  Synthetic
gtggagatttgcttacgagc

SEQ ID NO: 634 - DNA, 20 bp, Artificial sequence
  Synthetic
gaaccttggagaattgaata

SEQ ID NO: 635 - DNA, 20 bp, Artificial sequence
  Synthetic
gatagaaaagagcagctaca

SEQ ID NO: 636 - DNA, 20 bp, Artificial sequence
  Synthetic
gcaagaagaaactgctatta

SEQ ID NO: 637 - DNA, 20 bp, Artificial sequence
  Synthetic
gtaatgttgccgaagcaatt

SEQ ID NO: 638 - DNA, 20 bp, Artificial sequence
  Synthetic
gaatttcattacaggaagta

SEQ ID NO: 639 - DNA, 20 bp, Artificial sequence
  Synthetic
gaaaacacaccttatcacag

SEQ ID NO: 640 - DNA, 20 bp, Artificial sequence
  Synthetic
gttatctttgagagaacatt

SEQ ID NO: 641 - DNA, 20 bp, Artificial sequence
  Synthetic
gaactcttaaggttaataag

SEQ ID NO: 642 - DNA, 20 bp, Artificial sequence
  Synthetic
gaaccatccatcctcacctg

SEQ ID NO: 643 - DNA, 20 bp, Artificial sequence
  Synthetic
ggagatgcactggtaaaaag

SEQ ID NO: 644 - DNA, 20 bp, Artificial sequence
  Synthetic
gctcatctccacagccatcc

SEQ ID NO: 645 - DNA, 20 bp, Artificial sequence
  Synthetic
gagtggccggtgccatttct

SEQ ID NO: 646 - DNA, 20 bp, Artificial sequence
  Synthetic
gctactagcgaagaagaagg

SEQ ID NO: 647 - DNA, 20 bp, Artificial sequence
  Synthetic
gtaagcttaaaacattagta

SEQ ID NO: 648 - DNA, 20 bp, Artificial sequence
  Synthetic
gtttacaggaaggagaagga

SEQ ID NO: 649 - DNA, 20 bp, Artificial sequence
  Synthetic
gtaatatttgaggtatgaat

SEQ ID NO: 650 - DNA, 20 bp, Artificial sequence
  Synthetic
gatggctcacacttgctgta

SEQ ID NO: 651 - DNA, 20 bp, Artificial sequence
  Synthetic
gaaactgggaacaagcttta

SEQ ID NO: 652 - DNA, 20 bp, Artificial sequence
  Synthetic
gctaatgctttgcctacccc

SEQ ID NO: 653 - DNA, 20 bp, Artificial sequence
  Synthetic
gccttaccctcagtagtgaa

SEQ ID NO: 654 - DNA, 20 bp, Artificial sequence
  Synthetic
gaactgaagtttagaagtaa

SEQ ID NO: 655 - DNA, 20 bp, Artificial sequence
  Synthetic
gaaatatcatgatggtgaag

SEQ ID NO: 656 - DNA, 20 bp, Artificial sequence
  Synthetic
gtgttgattctgaacaagtt

SEQ ID NO: 657 - DNA, 20 bp, Artificial sequence
  Synthetic
ggccctgtcctggacataaa

SEQ ID NO: 658 - DNA, 20 bp, Artificial sequence
  Synthetic
gcacattctaatttgtggat

SEQ ID NO: 659 - DNA, 20 bp, Artificial sequence
  Synthetic
gaagttaacatggaattaaa

SEQ ID NO: 660 - DNA, 20 bp, Artificial sequence
  Synthetic
gtccttaggcttgcaatgct

SEQ ID NO: 661 - DNA, 20 bp, Artificial sequence
  Synthetic
gagagacaatttgggtctag

SEQ ID NO: 662 - DNA, 20 bp, Artificial sequence
  Synthetic
gttaaatccaatggattcct

SEQ ID NO: 663 - DNA, 20 bp, Artificial sequence
  Synthetic
gttctcaatttactgggatt

SEQ ID NO: 664 - DNA, 20 bp, Artificial sequence
  Synthetic
gcagctgtgctcaaaagacc

SEQ ID NO: 665 - DNA, 20 bp, Artificial sequence
  Synthetic
gaggcttagttgtaataatg

SEQ ID NO: 666 - DNA, 20 bp, Artificial sequence
  Synthetic
gcccctcaattccagtgtaa

SEQ ID NO: 667 - DNA, 20 bp, Artificial sequence
  Synthetic
gactggcaaatacaatttgc

SEQ ID NO: 668 - DNA, 20 bp, Artificial sequence
  Synthetic
gaatgcaatatagtgatctt

SEQ ID NO: 669 - DNA, 20 bp, Artificial sequence
  Synthetic
ggagagggtggtttaaaagc

SEQ ID NO: 670 - DNA, 20 bp, Artificial sequence
  Synthetic
gggtataccttaggaaagct

SEQ ID NO: 671 - DNA, 20 bp, Artificial sequence
  Synthetic
gatgcattcaatagctctgt

SEQ ID NO: 672 - DNA, 20 bp, Artificial sequence
  Synthetic
gggctaaataaagcaatgtt

SEQ ID NO: 673 - DNA, 20 bp, Artificial sequence
  Synthetic
gttattcataaattgtaagc

SEQ ID NO: 674 - DNA, 20 bp, Artificial sequence
  Synthetic
gtgacatagtgggatagccc

SEQ ID NO: 675 - DNA, 20 bp, Artificial sequence
  Synthetic
gggaacatttcttcataggg

SEQ ID NO: 676 - DNA, 20 bp, Artificial sequence
  Synthetic
ggtatgtgtccatatgtgtc

SEQ ID NO: 677 - DNA, 20 bp, Artificial sequence
  Synthetic
gaagaattaacacattgtct

SEQ ID NO: 678 - DNA, 20 bp, Artificial sequence
  Synthetic
gatgcctggttaacaattca

SEQ ID NO: 679 - DNA, 20 bp, Artificial sequence
  Synthetic
gccttaaagctcctatagaa

SEQ ID NO: 680 - DNA, 20 bp, Artificial sequence
  Synthetic
gggcccacatttatctctat

SEQ ID NO: 681 - DNA, 20 bp, Artificial sequence
  Synthetic
gcaggtgtctaaattcactc

SEQ ID NO: 682 - DNA, 20 bp, Artificial sequence
  Synthetic
gaacaataagtcaagcaagt

SEQ ID NO: 683 - DNA, 20 bp, Artificial sequence
  Synthetic
gggacaatctaaatgtccta

SEQ ID NO: 684 - DNA, 20 bp, Artificial sequence
  Synthetic
ggatataaaagcatacaaaa

SEQ ID NO: 685 - DNA, 20 bp, Artificial sequence
  Synthetic
gagtcaccccagggacaaac

SEQ ID NO: 686 - DNA, 20 bp, Artificial sequence
  Synthetic
ggaccctaagggaagcttga

SEQ ID NO: 687 - DNA, 20 bp, Artificial sequence
  Synthetic
gtactcactgatacacagct

SEQ ID NO: 688 - DNA, 20 bp, Artificial sequence
  Synthetic
gtttataaatattccgacta

SEQ ID NO: 689 - DNA, 20 bp, Artificial sequence
  Synthetic
ggtgactaggaagtttctgc

SEQ ID NO: 690 - DNA, 20 bp, Artificial sequence
  Synthetic
gacttagaaacagttaataa

SEQ ID NO: 691 - DNA, 20 bp, Artificial sequence
  Synthetic
gttattattgagttggtata

SEQ ID NO: 692 - DNA, 20 bp, Artificial sequence
  Synthetic
gaacactttcactgggaata

SEQ ID NO: 693 - DNA, 20 bp, Artificial sequence
  Synthetic
gggattctcctagaataaat

SEQ ID NO: 694 - DNA, 20 bp, Artificial sequence
  Synthetic
gcccacttatgcagtataag

SEQ ID NO: 695 - DNA, 20 bp, Artificial sequence
  Synthetic
gtgcataccaaattagtgtc

SEQ ID NO: 696 - DNA, 20 bp, Artificial sequence
  Synthetic
gtattcacagccaaaaagta

SEQ ID NO: 697 - DNA, 20 bp, Artificial sequence
  Synthetic
gttctgcttctaacatagta

SEQ ID NO: 698 - DNA, 20 bp, Artificial sequence
  Synthetic
ggaaaagctatgttaaacct

SEQ ID NO: 699 - DNA, 20 bp, Artificial sequence
  Synthetic
gtatctgcatattaaacaca

SEQ ID NO: 700 - DNA, 20 bp, Artificial sequence
  Synthetic
ggcccttaaaacatggaacc

SEQ ID NO: 701 - DNA, 20 bp, Artificial sequence
  Synthetic
gtagcctatgtcagaatgag

SEQ ID NO: 702 - DNA, 20 bp, Artificial sequence
  Synthetic
gagttgctagacagctacca

SEQ ID NO: 703 - DNA, 20 bp, Artificial sequence
  Synthetic
gaagcaacacagattctcac

SEQ ID NO: 704 - DNA, 20 bp, Artificial sequence
  Synthetic
ggttagcaaaattgcaagag

SEQ ID NO: 705 - DNA, 20 bp, Artificial sequence
  Synthetic
ggaacctggagaatgttaag

SEQ ID NO: 706 - DNA, 20 bp, Artificial sequence
  Synthetic
gtgttctcattcttcactca

SEQ ID NO: 707 - DNA, 20 bp, Artificial sequence
  Synthetic
gagtcacggtcaaacagtcg

SEQ ID NO: 708 - DNA, 20 bp, Artificial sequence
  Synthetic
gagaacatacacataatgac

SEQ ID NO: 709 - DNA, 20 bp, Artificial sequence
  Synthetic
gcttcaaatgtgtgtgcttc

SEQ ID NO: 710 - DNA, 20 bp, Artificial sequence
  Synthetic
gagaaattaactcactttat

SEQ ID NO: 711 - DNA, 20 bp, Artificial sequence
  Synthetic
gtatttaggctatgcttgaa

SEQ ID NO: 712 - DNA, 20 bp, Artificial sequence
  Synthetic
gtctttggaaacaaccatgt

SEQ ID NO: 713 - DNA, 20 bp, Artificial sequence
  Synthetic
gcccatcatgacaggacagg

SEQ ID NO: 714 - DNA, 20 bp, Artificial sequence
  Synthetic
ggtagagcaggggtattact

SEQ ID NO: 715 - DNA, 20 bp, Artificial sequence
  Synthetic
ggaagtgcatgcatgacctt

SEQ ID NO: 716 - DNA, 20 bp, Artificial sequence
  Synthetic
gttgaaatcaacataaggaa

SEQ ID NO: 717 - DNA, 20 bp, Artificial sequence
  Synthetic
ggggtggcactgggttaatt

SEQ ID NO: 718 - DNA, 20 bp, Artificial sequence
  Synthetic
gggcagatcgacaactgccg

SEQ ID NO: 719 - DNA, 20 bp, Artificial sequence
  Synthetic
gttgaattatgttacctcca

SEQ ID NO: 720 - DNA, 20 bp, Artificial sequence
  Synthetic
gaaaaatgacccatgattaa

SEQ ID NO: 721 - DNA, 20 bp, Artificial sequence
  Synthetic
ggtagagggataatgcactg

SEQ ID NO: 722 - DNA, 20 bp, Artificial sequence
  Synthetic
gaaagtcaagcagaggggca

SEQ ID NO: 723 - DNA, 20 bp, Artificial sequence
  Synthetic
ggagagaattaatcttattt

SEQ ID NO: 724 - DNA, 20 bp, Artificial sequence
  Synthetic
ggagacaccagtcacggagt

SEQ ID NO: 725 - DNA, 20 bp, Artificial sequence
  Synthetic
gagccaaagtggcaaagtgg

SEQ ID NO: 726 - DNA, 20 bp, Artificial sequence
  Synthetic
gtgggaggacaggcagcaga

SEQ ID NO: 727 - DNA, 20 bp, Artificial sequence
  Synthetic
gattaaagacttgcttagtt

SEQ ID NO: 728 - DNA, 20 bp, Artificial sequence
  Synthetic
gagcttatttgacatgttag

SEQ ID NO: 729 - DNA, 20 bp, Artificial sequence
  Synthetic
ggattaatgtagctgtaaat

SEQ ID NO: 730 - DNA, 20 bp, Artificial sequence
  Synthetic
gtaagagaccaagcccaagt

SEQ ID NO: 731 - DNA, 20 bp, Artificial sequence
  Synthetic
ggttcactgagtatgtgccc

SEQ ID NO: 732 - DNA, 20 bp, Artificial sequence
  Synthetic
ggatgcagccactctcagag

SEQ ID NO: 733 - DNA, 20 bp, Artificial sequence
  Synthetic
gaggtacctcacaatttgaa

SEQ ID NO: 734 - DNA, 20 bp, Artificial sequence
  Synthetic
gtatcaacagagtgtcagat

SEQ ID NO: 735 - DNA, 20 bp, Artificial sequence
  Synthetic
gtacctcaaagtgttccctg

SEQ ID NO: 736 - DNA, 20 bp, Artificial sequence
  Synthetic
ggcctctgtaagaggggagt

SEQ ID NO: 737 - DNA, 20 bp, Artificial sequence
  Synthetic
gatatataaagtaagtggag

SEQ ID NO: 738 - DNA, 20 bp, Artificial sequence
  Synthetic
gatccttattgctccattct

SEQ ID NO: 739 - DNA, 20 bp, Artificial sequence
  Synthetic
gaacttataaagtgcccaca

SEQ ID NO: 740 - DNA, 20 bp, Artificial sequence
  Synthetic
ggtagggttggaagggtaac

SEQ ID NO: 741 - DNA, 20 bp, Artificial sequence
  Synthetic
gtgatgcatagcatagtttc

SEQ ID NO: 742 - DNA, 20 bp, Artificial sequence
  Synthetic
gggaggcaacctgtccctgc

SEQ ID NO: 743 - DNA, 20 bp, Artificial sequence
  Synthetic
ggtacaatagatgcctgaaa

SEQ ID NO: 744 - DNA, 20 bp, Artificial sequence
  Synthetic
gggagtgactcagctacatg

SEQ ID NO: 745 - DNA, 20 bp, Artificial sequence
  Synthetic
ggtcatgatgccactgggag

SEQ ID NO: 746 - DNA, 20 bp, Artificial sequence
  Synthetic
gaccagtaagattaaaaatg

SEQ ID NO: 747 - DNA, 20 bp, Artificial sequence
  Synthetic
ggcactggtttgtgcacttc

SEQ ID NO: 748 - DNA, 20 bp, Artificial sequence
  Synthetic
gaaatattcaagtttatgag

SEQ ID NO: 749 - DNA, 20 bp, Artificial sequence
  Synthetic
gtttgcagcacacaggtaga

SEQ ID NO: 750 - DNA, 20 bp, Artificial sequence
  Synthetic
gtttggtacagtataaccaa

SEQ ID NO: 751 - DNA, 20 bp, Artificial sequence
  Synthetic
gatcataacagaagctccaa

SEQ ID NO: 752 - DNA, 20 bp, Artificial sequence
  Synthetic
gcaagagcaattctcaggct

SEQ ID NO: 753 - DNA, 20 bp, Artificial sequence
  Synthetic
gggccatggaaaacagccca

SEQ ID NO: 754 - DNA, 20 bp, Artificial sequence
  Synthetic
gtgttatgactttaaagtta

SEQ ID NO: 755 - DNA, 20 bp, Artificial sequence
  Synthetic
gcaggtcaaaagctctagac

SEQ ID NO: 756 - DNA, 20 bp, Artificial sequence
  Synthetic
gaaacctaaacaatagctcc

SEQ ID NO: 757 - DNA, 20 bp, Artificial sequence
  Synthetic
gccaagtggactagaagccg

SEQ ID NO: 758 - DNA, 20 bp, Artificial sequence
  Synthetic
gtgtcatcatgctaagtaat

SEQ ID NO: 759 - DNA, 20 bp, Artificial sequence
  Synthetic
gctctagattagttggctta

SEQ ID NO: 760 - DNA, 20 bp, Artificial sequence
  Synthetic
gacctctaattcacagagag

SEQ ID NO: 761 - DNA, 20 bp, Artificial sequence
  Synthetic
gactgagggtggataatcca

SEQ ID NO: 762 - DNA, 20 bp, Artificial sequence
  Synthetic
gagtcgaatgtaagaaattc

SEQ ID NO: 763 - DNA, 20 bp, Artificial sequence
  Synthetic
gatatgagagataattaaag

SEQ ID NO: 764 - DNA, 20 bp, Artificial sequence
  Synthetic
gaatacctacccattagtga

SEQ ID NO: 765 - DNA, 20 bp, Artificial sequence
  Synthetic
gtgttaagtagggaatatac

SEQ ID NO: 766 - DNA, 20 bp, Artificial sequence
  Synthetic
gagaaatgaggcgcttgtta

SEQ ID NO: 767 - DNA, 20 bp, Artificial sequence
  Synthetic
gattcacttagttgctcccc

SEQ ID NO: 768 - DNA, 20 bp, Artificial sequence
  Synthetic
gaatatgagctcctaacata

SEQ ID NO: 769 - DNA, 20 bp, Artificial sequence
  Synthetic
gtactcagcagaaacaaagg

SEQ ID NO: 770 - DNA, 20 bp, Artificial sequence
  Synthetic
gtgtacataaacaaaaagtt

SEQ ID NO: 771 - DNA, 20 bp, Artificial sequence
  Synthetic
gcaggtgcaatatttagtag

SEQ ID NO: 772 - DNA, 20 bp, Artificial sequence
  Synthetic
gtaaggccatgacaccaatt

SEQ ID NO: 773 - DNA, 20 bp, Artificial sequence
  Synthetic
gtcttaggtgcacaattccc

SEQ ID NO: 774 - DNA, 20 bp, Artificial sequence
  Synthetic
gtgttatctttcactcatat

SEQ ID NO: 775 - DNA, 20 bp, Artificial sequence
  Synthetic
gatttaagtcctccatgctt

SEQ ID NO: 776 - DNA, 20 bp, Artificial sequence
  Synthetic
gatttgacatgctttaataa

SEQ ID NO: 777 - DNA, 20 bp, Artificial sequence
  Synthetic
gtttccaggtgactcagtta

SEQ ID NO: 778 - DNA, 20 bp, Artificial sequence
  Synthetic
ggtctgtgtgtggatttcca

SEQ ID NO: 779 - DNA, 20 bp, Artificial sequence
  Synthetic
gtcaagccttatgcaatttc

SEQ ID NO: 780 - DNA, 20 bp, Artificial sequence
  Synthetic
gtcactggagaagcaacttc

SEQ ID NO: 781 - DNA, 20 bp, Artificial sequence
  Synthetic
gagactaaatgcgggaaaga

SEQ ID NO: 782 - DNA, 20 bp, Artificial sequence
  Synthetic
gaactaatcaatgtgcatca

SEQ ID NO: 783 - DNA, 20 bp, Artificial sequence
  Synthetic
ggcagccctaaggcagtcac

SEQ ID NO: 784 - DNA, 20 bp, Artificial sequence
  Synthetic
gggattgttaatgtccaagc

SEQ ID NO: 785 - DNA, 20 bp, Artificial sequence
  Synthetic
gcataaacattcatgagttt

SEQ ID NO: 786 - DNA, 20 bp, Artificial sequence
  Synthetic
gcactcacggagtgctaggg

SEQ ID NO: 787 - DNA, 20 bp, Artificial sequence
  Synthetic
gtgcttaatatgaatgctgg

SEQ ID NO: 788 - DNA, 20 bp, Artificial sequence
  Synthetic
ggaacatgaaaataacgttg

SEQ ID NO: 789 - DNA, 20 bp, Artificial sequence
  Synthetic
gtgacttcatttgatttcac

SEQ ID NO: 790 - DNA, 20 bp, Artificial sequence
  Synthetic
gccatccaccatgctatcaa

SEQ ID NO: 791 - DNA, 20 bp, Artificial sequence
  Synthetic
gagaatggagctgaaaatac

SEQ ID NO: 792 - DNA, 20 bp, Artificial sequence
  Synthetic
gcttgctctgtatgactgtc

SEQ ID NO: 793 - DNA, 20 bp, Artificial sequence
  Synthetic
gtcatcaggataaatcagcg

SEQ ID NO: 794 - DNA, 20 bp, Artificial sequence
  Synthetic
gtcttagtcagggaaggagt

SEQ ID NO: 795 - DNA, 20 bp, Artificial sequence
  Synthetic
ggatctcaagagctacctaa

SEQ ID NO: 796 - DNA, 20 bp, Artificial sequence
  Synthetic
gaaattacatccctagatag

SEQ ID NO: 797 - DNA, 20 bp, Artificial sequence
  Synthetic
gaagcaaaactacctttgtt

SEQ ID NO: 798 - DNA, 20 bp, Artificial sequence
  Synthetic
gcttcatctggggtgaaacc

SEQ ID NO: 799 - DNA, 20 bp, Artificial sequence
  Synthetic
gcattactaaccatggaaag

SEQ ID NO: 800 - DNA, 20 bp, Artificial sequence
  Synthetic
gtgggtcattcaagtggagc

SEQ ID NO: 801 - DNA, 20 bp, Artificial sequence
  Synthetic
gttccataagtggaagcgtt

SEQ ID NO: 802 - DNA, 20 bp, Artificial sequence
  Synthetic
gaaataggaagggaatataa

SEQ ID NO: 803 - DNA, 20 bp, Artificial sequence
  Synthetic
gtaacactcagcagctgaga

SEQ ID NO: 804 - DNA, 20 bp, Artificial sequence
  Synthetic
gctattccaggagaacacat

SEQ ID NO: 805 - DNA, 20 bp, Artificial sequence
  Synthetic
gtgttgataacagaagatcc

SEQ ID NO: 806 - DNA, 20 bp, Artificial sequence
  Synthetic
ggatcacatatacatgcctg

SEQ ID NO: 807 - DNA, 20 bp, Artificial sequence
  Synthetic
gtcaaactcttcaatattct

SEQ ID NO: 808 - DNA, 20 bp, Artificial sequence
  Synthetic
gcaacttgaactccaactta

SEQ ID NO: 809 - DNA, 20 bp, Artificial sequence
  Synthetic
gagactgaatataagatgta

SEQ ID NO: 810 - DNA, 20 bp, Artificial sequence
  Synthetic
gtgtcaaaaaacctcagaaa

SEQ ID NO: 811 - DNA, 20 bp, Artificial sequence
  Synthetic
gttaggaagtattcggagtt

SEQ ID NO: 812 - DNA, 20 bp, Artificial sequence
  Synthetic
gtatcaagtaaataggtgga

SEQ ID NO: 813 - DNA, 20 bp, Artificial sequence
  Synthetic
gtaaagcaacaggtaattaa

SEQ ID NO: 814 - DNA, 20 bp, Artificial sequence
  Synthetic
gatgtttattgtagggcatg

SEQ ID NO: 815 - DNA, 20 bp, Artificial sequence
  Synthetic
gaccactcaatttatatatt

SEQ ID NO: 816 - DNA, 20 bp, Artificial sequence
  Synthetic
ggccattatttgttgatcat

SEQ ID NO: 817 - DNA, 20 bp, Artificial sequence
  Synthetic
ggagaaactggatttaaaga

SEQ ID NO: 818 - DNA, 20 bp, Artificial sequence
  Synthetic
gtctacagaccacagaagaa

SEQ ID NO: 819 - DNA, 20 bp, Artificial sequence
  Synthetic
ggtatcccttaagaatttaa

SEQ ID NO: 820 - DNA, 20 bp, Artificial sequence
  Synthetic
ggtagattaatattctggaa

SEQ ID NO: 821 - DNA, 20 bp, Artificial sequence
  Synthetic
gtagttatccaaggtaacag

SEQ ID NO: 822 - DNA, 20 bp, Artificial sequence
  Synthetic
ggatttgcgcaggtccctct

SEQ ID NO: 823 - DNA, 20 bp, Artificial sequence
  Synthetic
gcatgttagccagcagaaca

SEQ ID NO: 824 - DNA, 20 bp, Artificial sequence
  Synthetic
gtcacctaaaacgatgtatg

SEQ ID NO: 825 - DNA, 20 bp, Artificial sequence
  Synthetic
gatactaatcaataagtggg

SEQ ID NO: 826 - DNA, 20 bp, Artificial sequence
  Synthetic
gaaggttatgggaggggtac

SEQ ID NO: 827 - DNA, 20 bp, Artificial sequence
  Synthetic
gcagaaagtgatctttacat

SEQ ID NO: 828 - DNA, 20 bp, Artificial sequence
  Synthetic
gaagaggtttaggttgtcag

SEQ ID NO: 829 - DNA, 20 bp, Artificial sequence
  Synthetic
gagccacagttagagtaact

SEQ ID NO: 830 - DNA, 20 bp, Artificial sequence
  Synthetic
gtattggctagttaagtgca

SEQ ID NO: 831 - DNA, 20 bp, Artificial sequence
  Synthetic
ggtcaccttaaaaacatcta

SEQ ID NO: 832 - DNA, 20 bp, Artificial sequence
  Synthetic
gtgcatttgggtattagatt

SEQ ID NO: 833 - DNA, 20 bp, Artificial sequence
  Synthetic
gaataatagctatggctgct

SEQ ID NO: 834 - DNA, 20 bp, Artificial sequence
  Synthetic
gggcattgcctgtttaatct

SEQ ID NO: 835 - DNA, 20 bp, Artificial sequence
  Synthetic
gactttgtcactaacacgca

SEQ ID NO: 836 - DNA, 20 bp, Artificial sequence
  Synthetic
gtaagcatgtacgaagtaac

SEQ ID NO: 837 - DNA, 20 bp, Artificial sequence
  Synthetic
gtttgccttccagataggag

SEQ ID NO: 838 - DNA, 20 bp, Artificial sequence
  Synthetic
gggagtgtatgttcattgga

SEQ ID NO: 839 - DNA, 20 bp, Artificial sequence
  Synthetic
gggtgactactggttgcttt

SEQ ID NO: 840 - DNA, 20 bp, Artificial sequence
  Synthetic
gttaaacctgtttatgctct

SEQ ID NO: 841 - DNA, 20 bp, Artificial sequence
  Synthetic
ggattctgaattaattgtag

SEQ ID NO: 842 - DNA, 20 bp, Artificial sequence
  Synthetic
gattctatagtctatagtta

SEQ ID NO: 843 - RNA, 23 nt, Artificial sequence
  Synthetic
aaacaugaggauuacccaugucg

SEQ ID NO: 844 - DNA, 17 bp, ARTIFICIAL sequence
  Synthetic
  misc_feature (1)..(4): n is a, c, g, or t
  misc_feature (6)..(7): n is a, c, g, or t
  misc_feature (9)..(9): n is a, c, g, or t
  misc_feature (12)..(17): n is a, c, g, or t
nnnnannanyannnnnn

SEQ ID NO: 845 - protein, 130 aa, Artificial sequence
  Synthetic
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 846 - protein, 129 aa, Artificial sequence
  Synthetic
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 847 - DNA, 20 bp, ARTIFICIAL sequence
  Synthetic
aaaaacccgcgctggttcac

SEQ ID NO: 848 - DNA, 20 bp, ARTIFICIAL sequence
  Synthetic
aacaaccaccctggccttca

SEQ ID NO: 849 - DNA, 20 bp, ARTIFICIAL sequence
  Synthetic
aacatggtgtatcagtacaa

SEQ ID NO: 850 - DNA, 20 bp, ARTIFICIAL sequence
  Synthetic
aaggtagttattataatata

SEQ ID NO: 851 - DNA, 20 bp, ARTIFICIAL sequence
  Synthetic
aagtacattccaaatgactt

SEQ ID NO: 852 - DNA, 20 bp, ARTIFICIAL sequence
  Synthetic
aatctatgagcttcgaaata

SEQ ID NO: 853 - DNA, 20 bp, ARTIFICIAL sequence
  Synthetic
accacgtgcgggaggatggc

SEQ ID NO: 854 - DNA, 20 bp, ARTIFICIAL sequence
  Synthetic
acctgctaggcaccatggct

SEQ ID NO: 855 - DNA, 20 bp, ARTIFICIAL sequence
  Synthetic
acgtagtagaggcctggaaa

SEQ ID NO: 856 - DNA, 20 bp, ARTIFICIAL sequence
  Synthetic
acgtggacagtgaagggaac

SEQ ID NO: 857 - DNA, 20 bp, ARTIFICIAL sequence
  Synthetic
agaaggtggcccctgaaatc

SEQ ID NO: 858 - DNA, 20 bp, ARTIFICIAL sequence
  Synthetic
agaccatcactgagactccc

SEQ ID NO: 859 - DNA, 20 bp, ARTIFICIAL sequence
  Synthetic
agagccagaacctacagaga

SEQ ID NO: 860 - DNA, 20 bp, ARTIFICIAL sequence
  Synthetic
agaggatcggcaacatggca

SEQ ID NO: 861 - DNA, 20 bp, ARTIFICIAL sequence
  Synthetic
agcctggccgcgccaggctg

SEQ ID NO: 862 - DNA, 20 bp, ARTIFICIAL sequence
  Synthetic
agcgcgggtttttcggactt

SEQ ID NO: 863 - DNA, 20 bp, ARTIFICIAL sequence
  Synthetic
agctgactccagggctacag

SEQ ID NO: 864 - DNA, 20 bp, ARTIFICIAL sequence
  Synthetic
agctgcatccaccctctttc

SEQ ID NO: 865 - DNA, 20 bp, ARTIFICIAL sequence
  Synthetic
aggcatctctgtaggtggct

SEQ ID NO: 866 - DNA, 20 bp, ARTIFICIAL sequence
  Synthetic
agtcaacctctaccacgtgc

SEQ ID NO: 867 - DNA, 20 bp, ARTIFICIAL sequence
  Synthetic
agtgaagggaaccggatttc

SEQ ID NO: 868 - DNA, 20 bp, ARTIFICIAL sequence
  Synthetic
agtggagcaggcctatgatc

SEQ ID NO: 869 - DNA, 20 bp, ARTIFICIAL sequence
  Synthetic
atccgctgcgcccccagcca

SEQ ID NO: 870 - DNA, 20 bp, ARTIFICIAL sequence
  Synthetic
atctgctggatctaggtcca

SEQ ID NO: 871 - DNA, 20 bp, ARTIFICIAL sequence
  Synthetic
atctgtggctgggataagag

SEQ ID NO: 872 - DNA, 20 bp, ARTIFICIAL sequence
  Synthetic
atgcatatggggtcatggat

SEQ ID NO: 873 - DNA, 20 bp, ARTIFICIAL sequence
  Synthetic
atttcgattcctggctcttc

SEQ ID NO: 874 - DNA, 20 bp, ARTIFICIAL sequence
  Synthetic
caaaggcatggggctgtcca

SEQ ID NO: 875 - DNA, 20 bp, ARTIFICIAL sequence
  Synthetic
caacctctaccacgtgcggg

SEQ ID NO: 876 - DNA, 20 bp, ARTIFICIAL sequence
  Synthetic
caagtccgaaaaacccgcgc

SEQ ID NO: 877 - DNA, 20 bp, ARTIFICIAL sequence
  Synthetic
caccatggctgggggcgcag

SEQ ID NO: 878 - DNA, 20 bp, ARTIFICIAL sequence
  Synthetic
caccatgttggcaagcagtt

SEQ ID NO: 879 - DNA, 20 bp, ARTIFICIAL sequence
  Synthetic
caccccagcctggcgcggcc

SEQ ID NO: 880 - DNA, 20 bp, ARTIFICIAL sequence
  Synthetic
caccttcttcaccgtctggg

SEQ ID NO: 881 - DNA, 20 bp, ARTIFICIAL sequence
  Synthetic
cacgtagtagaggcctggaa

SEQ ID NO: 882 - DNA, 20 bp, ARTIFICIAL sequence
  Synthetic
cagcgcgggtttttcggact

SEQ ID NO: 883 - DNA, 20 bp, ARTIFICIAL sequence
  Synthetic
cagctgcaactatgactcca

SEQ ID NO: 884 - DNA, 20 bp, ARTIFICIAL sequence
  Synthetic
cagcttctgggaacggctgt

SEQ ID NO: 885 - DNA, 20 bp, ARTIFICIAL sequence
  Synthetic
cagtcaacctctaccacgtg

SEQ ID NO: 886 - DNA, 20 bp, ARTIFICIAL sequence
  Synthetic
cataggcctgctccacttcc

SEQ ID NO: 887 - DNA, 20 bp, ARTIFICIAL sequence
  Synthetic
catagttgcagctgactcca

SEQ ID NO: 888 - DNA, 20 bp, ARTIFICIAL sequence
  Synthetic
catcctcccgcacgtggtag

SEQ ID NO: 889 - DNA, 20 bp, ARTIFICIAL sequence
  Synthetic
catggcgcttgccagcgtgt

SEQ ID NO: 890 - DNA, 20 bp, ARTIFICIAL sequence
  Synthetic
catgttggcaagcagtttgg

SEQ ID NO: 891 - DNA, 20 bp, ARTIFICIAL sequence
  Synthetic
ccacaccttgaaggccaggg

SEQ ID NO: 892 - DNA, 20 bp, ARTIFICIAL sequence
  Synthetic
ccacattgtcactggagact

SEQ ID NO: 893 - DNA, 20 bp, ARTIFICIAL sequence
  Synthetic
ccatgaagcatttcgattcc

SEQ ID NO: 894 - DNA, 20 bp, ARTIFICIAL sequence
  Synthetic
ccatggtgcctagcaggtat

SEQ ID NO: 895 - DNA, 20 bp, ARTIFICIAL sequence
  Synthetic
ccccagccatggtgcctagc

SEQ ID NO: 896 - DNA, 20 bp, ARTIFICIAL sequence
  Synthetic
ccgcgctggttcaccggtag

SEQ ID NO: 897 - DNA, 20 bp, ARTIFICIAL sequence
  Synthetic
cgcagcggattgcagcttct

SEQ ID NO: 898 - DNA, 20 bp, ARTIFICIAL sequence
  Synthetic
cgcgggtttttcggacttgg

SEQ ID NO: 899 - DNA, 20 bp, ARTIFICIAL sequence
  Synthetic
cgctaccggtgaaccagcgc

SEQ ID NO: 900 - DNA, 20 bp, ARTIFICIAL sequence
  Synthetic
cggattgcagcttctgggaa

SEQ ID NO: 901 - DNA, 20 bp, ARTIFICIAL sequence
  Synthetic
cgtgcagatctgctggatct

SEQ ID NO: 902 - DNA, 20 bp, ARTIFICIAL sequence
  Synthetic
cgtgttggagagaccgctac

SEQ ID NO: 903 - DNA, 20 bp, ARTIFICIAL sequence
  Synthetic
ctaacctcatctccctttcc

SEQ ID NO: 904 - DNA, 20 bp, ARTIFICIAL sequence
  Synthetic
ctatcaccttcttcaccgtc

SEQ ID NO: 905 - DNA, 20 bp, ARTIFICIAL sequence
  Synthetic
ctatgacctggaagtggagc

SEQ ID NO: 906 - DNA, 20 bp, ARTIFICIAL sequence
  Synthetic
ctattcctatgacctggaag

SEQ ID NO: 907 - DNA, 20 bp, ARTIFICIAL sequence
  Synthetic
ctctaccacgtgcgggagga

SEQ ID NO: 908 - DNA, 20 bp, ARTIFICIAL sequence
  Synthetic
ctctaccccctgaaagaggg

SEQ ID NO: 909 - DNA, 20 bp, ARTIFICIAL sequence
  Synthetic
ctctactacgtggacagtga

SEQ ID NO: 910 - DNA, 20 bp, ARTIFICIAL sequence
  Synthetic
ctgcaactatgactccatgg

SEQ ID NO: 911 - DNA, 20 bp, ARTIFICIAL sequence
  Synthetic
ctgcatccaccctctttcag

SEQ ID NO: 912 - DNA, 20 bp, ARTIFICIAL sequence
  Synthetic
ctgctaggcaccatggctgg

SEQ ID NO: 913 - DNA, 20 bp, ARTIFICIAL sequence
  Synthetic
ctgctccacttccaggtcat

SEQ ID NO: 914 - DNA, 20 bp, ARTIFICIAL sequence
  Synthetic
ctggctctgtgtatgcatat

SEQ ID NO: 915 - DNA, 20 bp, ARTIFICIAL sequence
  Synthetic
ctgtccacgtagtagaggcc

SEQ ID NO: 916 - DNA, 20 bp, ARTIFICIAL sequence
  Synthetic
cttatcccagccacagatca

SEQ ID NO: 917 - DNA, 20 bp, ARTIFICIAL sequence
  Synthetic
cttcactgtccacgtagtag

SEQ ID NO: 918 - DNA, 20 bp, ARTIFICIAL sequence
  Synthetic
ctttccaggcctctactacg

SEQ ID NO: 919 - DNA, 20 bp, ARTIFICIAL sequence
  Synthetic
ctttctgcccacactagaca

SEQ ID NO: 920 - DNA, 20 bp, ARTIFICIAL sequence
  Synthetic
gagatcaacccatacctgct

SEQ ID NO: 921 - DNA, 20 bp, ARTIFICIAL sequence
  Synthetic
gagcctggccgcgccaggct

SEQ ID NO: 922 - DNA, 20 bp, ARTIFICIAL sequence
  Synthetic
gatctacatgagaagtatag

SEQ ID NO: 923 - DNA, 20 bp, ARTIFICIAL sequence
  Synthetic
gatctgctggatctaggtcc

SEQ ID NO: 924 - DNA, 20 bp, ARTIFICIAL sequence
  Synthetic
gcaagcgccatgtctagtgt

SEQ ID NO: 925 - DNA, 20 bp, ARTIFICIAL sequence
  Synthetic
gcatatggggtcatggatcg

SEQ ID NO: 926 - DNA, 20 bp, ARTIFICIAL sequence
  Synthetic
gccacagatcatggtgccca

SEQ ID NO: 927 - DNA, 20 bp, ARTIFICIAL sequence
  Synthetic
gccaccttctctgtaggttc

SEQ ID NO: 928 - DNA, 20 bp, ARTIFICIAL sequence
  Synthetic
gccagaacctacagagaagg

SEQ ID NO: 929 - DNA, 20 bp, ARTIFICIAL sequence
  Synthetic
gccatggtgcctagcaggta

SEQ ID NO: 930 - DNA, 20 bp, ARTIFICIAL sequence
  Synthetic
gcgcagcggattgcagcttc

SEQ ID NO: 931 - DNA, 20 bp, ARTIFICIAL sequence
  Synthetic
gcgcgggtttttcggacttg

SEQ ID NO: 932 - DNA, 20 bp, ARTIFICIAL sequence
  Synthetic
gctccacaccttgaaggcca

SEQ ID NO: 933 - DNA, 20 bp, ARTIFICIAL sequence
  Synthetic
gctgactccagggctacagc

SEQ ID NO: 934 - DNA, 20 bp, ARTIFICIAL sequence
  Synthetic
gctgcatccaccctctttca

SEQ ID NO: 935 - DNA, 20 bp, ARTIFICIAL sequence
  Synthetic
gcttcatggaacaaccaccc

SEQ ID NO: 936 - DNA, 20 bp, ARTIFICIAL sequence
  Synthetic
ggcaagcgccatgtctagtg

SEQ ID NO: 937 - DNA, 20 bp, ARTIFICIAL sequence
  Synthetic
ggcggaactgttaagatcag

SEQ ID NO: 938 - DNA, 20 bp, ARTIFICIAL sequence
  Synthetic
ggctccacaccttgaaggcc

SEQ ID NO: 939 - DNA, 20 bp, ARTIFICIAL sequence
  Synthetic
ggctcgacgggccagatcat

SEQ ID NO: 940 - DNA, 20 bp, ARTIFICIAL sequence
  Synthetic
ggctgggataagagaggccc

SEQ ID NO: 941 - DNA, 20 bp, ARTIFICIAL sequence
  Synthetic
ggcttggtagatggctcgac

SEQ ID NO: 942 - DNA, 20 bp, ARTIFICIAL sequence
  Synthetic
gggctggctccacaccttga

SEQ ID NO: 943 - DNA, 20 bp, ARTIFICIAL sequence
  Synthetic
ggtccagggagtctcagtga

SEQ ID NO: 944 - DNA, 20 bp, ARTIFICIAL sequence
  Synthetic
ggtctgagcctggccgcgcc

SEQ ID NO: 945 - DNA, 20 bp, ARTIFICIAL sequence
  Synthetic
ggtgtatcagtacaaaggca

SEQ ID NO: 946 - DNA, 20 bp, ARTIFICIAL sequence
  Synthetic
ggttgcagcttaactcacca

SEQ ID NO: 947 - DNA, 20 bp, ARTIFICIAL sequence
  Synthetic
gtaagcacccgctgtagccc

SEQ ID NO: 948 - DNA, 20 bp, ARTIFICIAL sequence
  Synthetic
gtgaaccagcgcgggttttt

SEQ ID NO: 949 - DNA, 20 bp, ARTIFICIAL sequence
  Synthetic
gtgaagggaaccggatttca

SEQ ID NO: 950 - DNA, 20 bp, ARTIFICIAL sequence
  Synthetic
gtggctctaccccctgaaag

SEQ ID NO: 951 - DNA, 20 bp, ARTIFICIAL sequence
  Synthetic
gtgtatcagtacaaaggcat

SEQ ID NO: 952 - DNA, 20 bp, ARTIFICIAL sequence
  Synthetic
gttgactgcacctcctgagt

SEQ ID NO: 953 - DNA, 20 bp, ARTIFICIAL sequence
  Synthetic
tagatcagccacattgtcac

SEQ ID NO: 954 - DNA, 20 bp, ARTIFICIAL sequence
  Synthetic
tagcggtctctccaacacgc

SEQ ID NO: 955 - DNA, 20 bp, ARTIFICIAL sequence
  Synthetic
tatcaccttcttcaccgtct

SEQ ID NO: 956 - DNA, 20 bp, ARTIFICIAL sequence
  Synthetic
tcatagttgcagctgactcc

SEQ ID NO: 957 - DNA, 20 bp, ARTIFICIAL sequence
  Synthetic
tccagccatcctcccgcacg

SEQ ID NO: 958 - DNA, 20 bp, ARTIFICIAL sequence
  Synthetic
tccatgggcaccatgatctg

SEQ ID NO: 959 - DNA, 20 bp, ARTIFICIAL sequence
  Synthetic
tcggggctattcctatgacc

SEQ ID NO: 960 - DNA, 20 bp, ARTIFICIAL sequence
  Synthetic
tctactacgtggacagtgaa

SEQ ID NO: 961 - DNA, 20 bp, ARTIFICIAL sequence
  Synthetic
tctcagtgatggtctgagcc

SEQ ID NO: 962 - DNA, 20 bp, ARTIFICIAL sequence
  Synthetic
tctggctctgtgtatgcata

SEQ ID NO: 963 - DNA, 20 bp, ARTIFICIAL sequence
  Synthetic
tctgggaacggctgttggct

SEQ ID NO: 964 - DNA, 20 bp, ARTIFICIAL sequence
  Synthetic
tctgtaggtggcttggtaga

SEQ ID NO: 965 - DNA, 20 bp, ARTIFICIAL sequence
  Synthetic
tcttctgggacaccccagcc

SEQ ID NO: 966 - DNA, 20 bp, ARTIFICIAL sequence
  Synthetic
tgaagggaaccggatttcag

SEQ ID NO: 967 - DNA, 20 bp, ARTIFICIAL sequence
  Synthetic
tgagcctggccgcgccaggc

SEQ ID NO: 968 - DNA, 20 bp, ARTIFICIAL sequence
  Synthetic
tgagtaggcatctctgtagg

SEQ ID NO: 969 - DNA, 20 bp, ARTIFICIAL sequence
  Synthetic
tgatcttaacagttccgcca

SEQ ID NO: 970 - DNA, 20 bp, ARTIFICIAL sequence
  Synthetic
tgcatatggggtcatggatc

SEQ ID NO: 971 - DNA, 20 bp, ARTIFICIAL sequence
  Synthetic
tgcatccaccctctttcagg

SEQ ID NO: 972 - DNA, 20 bp, ARTIFICIAL sequence
  Synthetic
tgcctcccagacggtgaaga

SEQ ID NO: 973 - DNA, 20 bp, ARTIFICIAL sequence
  Synthetic
tgctgagaggatcggcaaca

SEQ ID NO: 974 - DNA, 20 bp, ARTIFICIAL sequence
  Synthetic
tgcttacattgcctcccaga

SEQ ID NO: 975 - DNA, 20 bp, ARTIFICIAL sequence
  Synthetic
tgcttgaaacctaagtcatt

SEQ ID NO: 976 - DNA, 20 bp, ARTIFICIAL sequence
  Synthetic
tggctctaccccctgaaaga

SEQ ID NO: 977 - DNA, 20 bp, ARTIFICIAL sequence
  Synthetic
tggctctgtgtatgcatatg

SEQ ID NO: 978 - DNA, 20 bp, ARTIFICIAL sequence
  Synthetic
tggcttggtagatggctcga

SEQ ID NO: 979 - DNA, 20 bp, ARTIFICIAL sequence
  Synthetic
tgggacaccccagcctggcg

SEQ ID NO: 980 - DNA, 20 bp, ARTIFICIAL sequence
  Synthetic
tgggggtcgtgcagatctgc

SEQ ID NO: 981 - DNA, 20 bp, ARTIFICIAL sequence
  Synthetic
tggggtgtcccagaagagcc

SEQ ID NO: 982 - DNA, 20 bp, ARTIFICIAL sequence
  Synthetic
tggttgcagcttaactcacc

SEQ ID NO: 983 - DNA, 20 bp, ARTIFICIAL sequence
  Synthetic
tgtgggtgtgctgagaggat

SEQ ID NO: 984 - DNA, 20 bp, ARTIFICIAL sequence
  Synthetic
tgtgtatgcatatggggtca

SEQ ID NO: 985 - DNA, 20 bp, ARTIFICIAL sequence
  Synthetic
tgttttgtgggtgtgctgag

SEQ ID NO: 986 - DNA, 20 bp, ARTIFICIAL sequence
  Synthetic
ttggaatgtacttgttttgt

SEQ ID NO: 987 - DNA, 20 bp, ARTIFICIAL sequence
  Synthetic
tttcgattcctggctcttct

SEQ ID NO: 988 - DNA, 20 bp, ARTIFICIAL sequence
  Synthetic
tttggaatgtacttgttttg

SEQ ID NO: 989 - DNA, 20 bp, ARTIFICIAL sequence
  Synthetic
tttgtactgatacaccatgt

(19) United States
(12) Patent Application Publication
(10) Pub. No.: US 2019/0309288 A1
(43) Pub. Date: Oct. 10, 2019

(54) TARGETED MUTAGENESIS

(71) Applicant: THE BOARD OF TRUSTEES OF THE LELAND STANFORD JUNIOR UNIVERSITY, Stanford, CA (US)

(72) Inventors: Gaelen Hess, Stanford, CA (US); Michael C. Bassik, Stanford, CA (US)

(21) Appl. No.: 16/325,873

(22) PCT Filed: Aug. 18, 2017
(86) PCT No.: PCT/US17/47624

§ 371 Date: Feb. 15, 2019

Related U.S. Application Data
(60) Provisional application No. 62/376,681, filed on Aug. 18, 2016.

(51) Int. Cl. C12N 15/10; C12N 9/22; C12N 9/78; C12N 15/11; C12N 15/90

(52) U.S. Cl. CPC C12N 15/1058; C12N 9/22; C12N 9/78; C12Y 305/04005; C12N 2800/80; C12N 15/907; C12N 2310/20; C12N 2320/13; C12N 15/11

(57) ABSTRACT

Provided herein is technology relating to the mutagenesis of nucleic acids, e.g., for directed evolution, and particularly, but not exclusively, to methods, compositions, and kits for producing nucleic acids and/or proteins comprising mutations and substitutions within specific target sequences.

This application claims priority to U.S. provisional patent application Ser. No. 62/376,681, filed Aug. 18, 2016, which is incorporated herein by reference in its entirety.

### STATEMENT REGARDING FEDERALLY SPONSORED RESEARCH OR DEVELOPMENT

This invention was made with government support under Grant Nos. S10RR025518-01, T32HG000044, ES016486, R01HG008150, and 1DP2HD084069-01, awarded by the National Institutes of Health; and by Grant No. DGE-114747, awarded by the National Science Foundation. The government has certain rights in the invention.

### FIELD

Provided herein is technology relating to the mutagenesis of nucleic acids, e.g., for directed evolution, and particularly, but not exclusively, to methods, compositions, and kits for producing nucleic acids and/or proteins comprising mutations and substitutions within specific target sequences.

### BACKGROUND

Directed evolution technologies employ mutation and selection to engineer biomolecules with enhanced, novel, or non-natural functions, such as improved antibodies (1), more efficient enzymes (2), or mutant proteins with altered activity (3).

However, extant technologies have limited capabilities to produce and maintain a diverse mutant population. For example, some current approaches comprise use of radiation and chemically-induced DNA damage to introduce mutations across an entire genome, but these approaches require maintaining a large number of cells for subsequent study because the majority of mutations are located outside the target of interest. In other extant approaches, diverse plasmid libraries are introduced into cells; however, proteins encoded by the plasmid libraries are often expressed at inappropriate levels for subsequent use and are expressed without normal, biologically relevant regulation. Further, the plasmid libraries used in current technologies have a limited size (e.g., limited total mutant diversity and/or limited size of the mutagenized target region) that restricts the potential for subsequent evolution experiments. Also, strategies for engineering biomolecules (e.g., nucleic acids and proteins) using extant directed evolution technologies have generally been implemented using bacteria, bacteriophage, and yeast because of current technological limitations of producing and maintaining sufficiently diverse libraries in a recombinant host for directed evolution (4-6).

However, mammalian proteins engineered in extant systems often change their behaviors when introduced into their native host environment. Accordingly, technologies for generating a diverse library of mutants in their native biological contexts are needed.

### SUMMARY

Accordingly, provided herein is a technology related to producing localized, diverse mutations at a specific genetic locus or at multiple specific genetic loci. The technology combines a modified biological mechanism for generating diversity at a genetic locus with sequence specificity provided by a modified CRISPR/Cas9 system.

The first feature of the technology is based on the exquisitely precise biological process of antibody maturation. In this process, B cells create point mutations in immunoglobulin (Ig) regions through the process of somatic hypermutation (SHM) (7, 8). SHM is mediated by an enzyme called activation induced cytidine deaminase (AID), which deaminates cytosine (C) to a uracil (U). Deamination of cytosine initiates a DNA repair response that introduces point mutations at the Ig locus at a rate of 10⁻³ bp (9). The process generates point mutations rather than insertions/deletions and favors transition mutations (pyrimidine to pyrimidine or purine to purine) over transversions (7). After deamination, mutations are generated in three ways: (1) a uracil-guanine (U-G) mismatch is misread to produce a (C>T) or (G>A) transition; (2) the U is removed by base excision repair and replaced by any base; or (3) an error-prone translesion polymerase is recruited through the mismatch repair pathway, generating transitions and transversions near the lesion (8).

The mechanisms by which SHM is regulated and targeted are not completely understood. For example, it has been proposed that sequence elements flanking the immunoglobulin locus are involved in SHM targeting (10). Also, it has been proposed that AID migrates with the RNA polymerase II complex during transcription of the Ig locus and mutates specific hotspot sequence motifs (11, 12). While cell lines that misregulate or overexpress AID have the mutagenic capacity to produce mutations for directed evolution (e.g., of fluorescent proteins (13, 14) and antibodies (15)), extant technologies create mutations throughout the genome (e.g., at numerous off-target sites) rather than at specific, defined genetic loci (e.g., at target sites).

The second feature of the technology is based on a modified CRISPR/Cas9 system. The CRISPR/Cas9 system provides for targeting proteins or other biomolecules to specific genomic loci using a modified Cas9 protein, e.g., catalytically inactive (“dead”) Cas9 (“dCas9”) protein. This approach has been used for both repression and activation of transcription (16-19) as well as for targeting fluorescent proteins (20, 21) and modifying enzymes (22-25) to particular genetic loci.

The technology provided herein comprises use of a dCas9 protein to target a deaminase (e.g., an AID, e.g., a hyperactive AID) to induce localized, diverse mutations at a genetic locus or multiple genetic loci. The present technology differs markedly from extant methods of using Cas9 for mutagenesis (25), which predominantly generate insertions and deletions (26-28) or that require homologous recombination to introduce mutations from a donor (29).

During the development of embodiments of the technology provided herein, data were collected indicating that AID-induced mutations are generated in cells that express AID constitutively or transiently. Furthermore, in some embodiments of the technology AID-induced mutations are targeted to multiple loci in the same cell. During the development of embodiments of the technology provided herein, the technology was used in protein engineering experiments to alter the absorption and/or emission spectra of genomically integrated wild-type GFP and to produce variants of PSMB5 that are resistant to bortezomib, a widely used chemotherapeutic drug. The technology produced mutations that have previously been observed in resistant cell lines and novel drug-resistant mutants that reveal new properties of PSMB5 and its interaction with bortezomib (see Table 7). Finally, during the development of embodiments of the technology provided herein, data were collected from experiments indicating that a hyperactive AID enzyme introduces mutations at a higher rate that the wild-type AID and that the hyperactive AID enzyme generates variants in protein coding regions and in non-protein coding regions, e.g., regulatory regions upstream of the transcription start site. The technology provides a novel targeted mutagenesis strategy for the engineering and evolution of new protein function in a normal cellular context.

Accordingly, provided herein is technology related to a composition for targeted mutagenesis of a nucleic acid, the composition comprising: a) an RNA comprising a scaffold sequence, a targeting sequence, and a binding sequence; b) a first protein that binds to the scaffold sequence to form a RNA-guided DNA binding complex; and c) a second protein that binds to the binding sequence and comprises a nucleic acid editing activity. For example, in some embodiments the RNA is an sgRNA, in some embodiments the binding sequence comprises a secondary structure that specifically interacts with the second protein, and in some embodiments the targeting sequence is complementary to a target site to be mutagenized. In particular embodiments, the first protein is a dCas9; in particular embodiments, the second protein comprises an MS2 protein; and, in some particular embodiments the second protein comprises a deaminase, e.g., an AID deaminase (e.g., a hyperactive AID deaminase such as, e.g., AIDΔ, AIDΔ, etc.). In some embodiments, the second protein is an MS2-AID fusion protein. Particular embodiments provide a composition wherein the binding sequence comprises a MS2-binding stem-loop structure. Related embodiments provide a composition wherein a plurality (e.g., 2, 3, 4, 5, 6 or more) of the second protein binds to the binding sequence. Further, related embodiments provide a composition wherein the RNA comprises a plurality (e.g., 2, 3, 4, 5, 6 or more) of binding sequences. In some embodiments, the composition comprises an RNA comprising a plurality (e.g., 2, 3, 4, 5, 6 or more) of binding sequences and wherein a plurality (e.g., 2, 3, 4, 5, 6 or more) of the second protein binds to each binding sequence. In some embodiments, the composition comprises an RNA comprising a plurality (e.g., 2, 3, 4, 5, 6 or more) of binding sequences, the second protein comprises a deaminase, e.g., an AID deaminase (e.g., a hyperactive AID deaminase such as, e.g., AIDΔ, AID*Δ, etc.), and wherein a plurality (e.g., 2, 3, 4, 5, 6 or more) of the second protein binds to each binding sequence. Said embodiments provide a composition for producing multiple mutations in a nucleic acid over a large defined region of a nucleic acid, e.g., a region of 10, 20, 30, 40, 50, 60, 70, 80, 90, 100, 200, 300, 400, 500, 600, 700, 800, 900, or 1000 or more base pairs in a nucleic acid. Some particular embodiments provide a composition wherein the binding sequence comprises a primary structure according to SEQ ID NO: 844 and/or wherein the MS2 protein comprises a primary structure according to SEQ ID NO: 846 and/or wherein the first protein comprises a sequence according to SEQ ID NO: 1.

The composition finds use in producing mutations in a nucleic acid. Accordingly, the technology provides compositions comprising: a) an RNA comprising a scaffold sequence, a targeting sequence, and a binding sequence; b) a first protein that binds to the scaffold sequence to form a RNA-guided DNA binding complex; c) a second protein that binds to the binding sequence and comprises a nucleic acid editing activity; and d) a nucleic acid comprising a target site. Embodiments of the technology comprise a composition having a nucleic acid editing activity that creates mutations in the nucleic acid within 20 bp of the target site. Embodiments of the technology comprise a composition having a nucleic acid editing activity that creates mutations in the nucleic acid within 50 bp of the target site. Embodiments of the technology comprise a composition having a nucleic acid editing activity that creates mutations in the nucleic acid within 100 bp of the target site. Embodiments of the technology comprise a composition having a nucleic acid editing activity that creates mutations in the nucleic acid within 1000 bp or more of the target site.

Embodiments of the technology comprise a composition having a nucleic acid editing activity that produces mutations at a rate of approximately 1 mutation per 1000 bp. Embodiments of the technology comprise a composition having a nucleic acid editing activity that produces mutations at a rate of approximately 1 mutation per 2000 bp. In some embodiments, the nucleic acid editing activity creates more than one mutation in a single nucleic acid. In some embodiments, the nucleic acid editing activity creates more than one mutation within a region of approximately 100 bp in a single nucleic acid. In some embodiments, the nucleic acid editing activity creates mutations in a coding region and/or in a non-coding region.

In related embodiments, the technology provides a composition for simultaneous targeted mutagenesis of multiple genetic loci in the same cell, the composition comprising: a) a first RNA comprising a scaffold sequence, a first targeting sequence, and a binding sequence; b) a second RNA comprising said scaffold sequence, a second targeting sequence, and said binding sequence; c) a first protein that binds to the scaffold sequence to form a RNA-guided DNA binding complex; and d) a second protein that binds to the binding sequence and comprises a nucleic acid editing activity. For example, embodiments provide a composition for simultaneous targeted mutagenesis of multiple genetic loci in the same cell, the composition comprising: a) a first RNA comprising a scaffold sequence, a first targeting sequence, and a binding sequence; b) a second RNA comprising said scaffold sequence, a second targeting sequence, and said binding sequence; c) a first protein that binds to the scaffold sequence to form a RNA-guided DNA binding complex; and d) a second protein that binds to the binding sequence and comprises a nucleic acid editing activity, wherein the first targeting sequence is complementary to a first target site and the second targeting sequence is complementary to a second target site.

Some embodiments provide a kit for directed mutagenesis comprising a composition as described herein. For example, kit embodiments provide a kit for directed mutagenesis comprising: a) an RNA comprising a scaffold sequence, a targeting sequence, and a binding sequence; b) a first protein that binds to the scaffold sequence to form a RNA-guided DNA binding complex; and c) a second protein that binds to the binding sequence and comprises a nucleic acid editing activity. In some embodiments kit comprise an RNA that is an sgRNA; in some embodiments the binding sequence comprises a secondary structure that specifically interacts with the second protein, and in some embodiments the targeting sequence is complementary to a target site to be mutagenized. In particular kit embodiments, the first protein is a dCas9; in particular kit embodiments, the second protein comprises an MS2 protein; and, in some particular kit embodiments the second protein comprises a deaminase, e.g., an AID deaminase (e.g., a hyperactive AID deaminase such as, e.g., AIDΔ, AID*Δ, etc.). In some kit embodiments, the second protein is an MS2-AID fusion protein. Particular kit embodiments provide a composition wherein the binding sequence comprises a MS2-binding stem-loop structure. Related kit embodiments comprise a composition wherein a plurality (e.g., 2, 3, 4, 5, 6 or more) of the second protein binds to the binding sequence. Further, related kit embodiments comprise a composition wherein the RNA comprises a plurality (e.g., 2, 3, 4, 5, 6 or more) of binding sequences. In some kit embodiments, a composition comprises an RNA comprising a plurality (e.g., 2, 3, 4, 5, 6 or more) of binding sequences and wherein a plurality (e.g., 2, 3, 4, 5, 6 or more) of the second protein binds to each binding sequence. In some kit embodiments, a composition comprises an RNA comprising a plurality (e.g., 2, 3, 4, 5, 6 or more) of binding sequences, the second protein comprises a deaminase, e.g., an AID deaminase (e.g., a hyperactive AID deaminase such as, e.g., AIDΔ, AIDΔ, etc.), and wherein a plurality (e.g., 2, 3, 4, 5, 6 or more) of the second protein binds to each binding sequence. Said kit embodiments provide a kit for producing multiple mutations in a nucleic acid over a large region of a nucleic acid, e.g., a region of 10, 20, 30, 40, 50, 60, 70, 80, 90, 100, 200, 300, 400, 500, 600, 700, 800, 900, or 1000 or more base pairs in a nucleic acid. Some particular kit embodiments provide a composition wherein the binding sequence comprises a primary structure according to SEQ ID NO: 844 and/or wherein the MS2 protein comprises a primary structure according to SEQ ID NO: 846 and/or wherein the first protein comprises a sequence according to SEQ ID NO: 1. Kit embodiments find use in producing mutants for directed evolution, e.g., by using a screening method or applying selection upon a mutant pool produced by the kits to identify products of directed evolution (e.g., nucleic acids, proteins, and/or cells or organisms) having desired (e.g., improved) qualities relative to wild-type or input nucleic acids or the expression products of wild-type or input nucleic acids.

Some embodiments provide a method for producing a product of directed evolution, the method comprising: a) producing a mutant pool by contacting an input nucleic acid comprising a target site to be mutagenized with a composition comprising: 1) an RNA comprising a scaffold sequence, a targeting sequence complementary to the target site, and a binding sequence; 2) a first protein that binds to the scaffold sequence to form a RNA-guided DNA binding complex; and 3) a second protein that binds to the binding sequence and comprises a nucleic acid editing activity; and b) screening or selecting the mutant pool to identify a product of directed evolution. For example, some embodiments provide a method wherein the product of directed evolution is a mutant nucleic acid comprising at least one mutation relative to the input nucleic acid, wherein the product of directed evolution is a protein or nucleic acid expressed from a mutant nucleic acid comprising at least one mutation relative to the input nucleic acid, and/or wherein the product of directed evolution is a cell or organism expressing a mutant nucleic acid comprising at least one mutation relative to the input nucleic acid or expressing a protein expressed from a mutant nucleic acid comprising at least one mutation relative to the input nucleic acid. In some embodiments, the technology provides a method of directed evolution wherein the product of directed evolution is a eukaryotic cell or a eukaryotic organism expressing a mutant nucleic acid comprising at least one mutation relative to the input nucleic acid or expressing a protein expressed from a mutant nucleic acid comprising at least one mutation relative to the input nucleic acid or wherein the product of directed evolution is a mammalian cell or a mammalian organism expressing a mutant nucleic acid comprising at least one mutation relative to the input nucleic acid or expressing a protein expressed from a mutant nucleic acid comprising at least one mutation relative to the input nucleic acid.

In certain embodiments, the RNA, first protein, and second protein are expressed in a cell comprising the nucleic acid comprising the target site. In some embodiments, the target site is a genetic locus in a genome.

In some embodiments, the mutant pool comprises at least 10³ mutants, at least 10⁴ mutants, at least 10⁵ mutants, at least 10⁶ mutants, or at least 10⁷ mutants.

In some embodiments, multiple rounds of mutant production and screening/selection are performed, e.g., to enrich the mutant population for nucleic acids and/or expression products of nucleic acids and/or cells or organisms comprising nucleic acids having desirable (e.g., improved) characteristics. Accordingly, the technology provides a method for producing a product of directed evolution, the method comprising repeating the above described method multiple times, e.g., a method wherein the product of directed evolution of a first cycle (e.g., cycle N) is used to provide the input nucleic acid of a subsequent cycle (e.g., cycle N+1).

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a schematic drawing of an embodiment of the technology. The drawing shows a dCas9 protein, a sgRNA comprising a plurality (e.g., 2) of MS2-binding hairpins, and a plurality of MS2-AID (e.g., AIDΔ) fusion proteins that specifically interact with the MS2-binding hairpins. The dCas9/sgRNA directs the AIDΔ to a specific genetic locus, where the deaminase induces local DNA damage, which in turn introduces mutations in the nucleic acid.

FIG. 2 is schematic drawing of three AID variants: 1) wild-type AID; 2) a truncated version lacking the last three amino acids (AIDΔ), which is a mutant protein without a functional nuclear export signal (NES) and having increasing SHM activity; and 3) a catalytically inactive truncated version (AIDΔDead). The NLS, NES, deaminase domain, truncations, and inactivating mutations H56R and E58Q are indicated.

FIG. 3 is a plot showing the enrichment of mutations in GFP. K562 cells containing dCas9, GFP, and mCherry were transfected with indicated combinations of MS2-AID, MS2-AIDΔ, or MS2-AIDΔDead and either sgGFP.1 or sgNegCtrl. GFP and mCherry fluorescence of the cells were measured by flow cytometry as a proxy for mutation rate. Cells were sorted for low GFP expression and the GFP locus was sequenced to identify mutations. MS2-AIDΔ sgNegCtrl and MS2-AIDΔDead; sgGFP.1 were essentially at baseline in the plot; MS2-AIDΔ; sgGFP.1 showed enrichment levels up to over 500× at particular mutational hotspots.

FIG. 4 shows plots indicating that the technology produces on-target mutations with minimized off-target effects. Cells were infected with indicated combinations of MS2-AIDΔ or MS2-34 AIDΔDead and sgGFP.1 or sgNegCtrl and the GFP and mCherry fluorescence of the cells was measured by flow cytometry as a proxy for mutation rate. Plots show the percentage of non-fluorescent cells resulting from the mutagenesis.

FIG. 5 shows plots indicating the locations of mutations in the experiments described in FIG. 4. Cells were infected with indicated combinations of MS2-AIDΔ or MS2-34 AIDΔDead and sgGFP.1 or sgNegCtrl. GFP and mCherry loci of the infected cells were sequenced and the enrichment of mutation was calculated at each base position for three replicate experiments. Error bars represent standard error.

FIG. 6 is a schematic map of sgRNAs tiling the GFP locus.

FIG. 7 shows data from experiments in which 12 guides targeting GFP (FIG. 6) were infected into cells expressing dCas9, MS2-AIDΔ, GFP, and mCherry. The targeting locations of the guides in the GFP locus are shown in the schematic drawing in FIG. 6. The GFP locus was sequenced for each sample. Enrichment of mutation relative to the position of the PAM of the sgRNAs is shown on the lower panel. The direction of transcription was defined as the positive direction as indicated by the arrow. The data indicate that the technology generates targeted mutations.

FIG. 8 is a series of plots showing the mutation enrichment for a series of sgRNA tiled across GFP (FIG. 6). sgRNAs targeting GFP were integrated into cells expressing dCas9, MS2-AIDΔ, GFP, and mCherry, and the GFP locus was sequenced. Enrichment of mutations at each base position is shown for three replicates of each sgRNA.

FIG. 9 is box plot indicating the frequency of mutated reads observed in the respective hotspot of each sgRNA shown in FIG. 6. The median value for the conditions is listed above each box.

FIG. 10 shows data for the directed evolution of bortezomib resistant mutations in PSMB5. Libraries targeting the exons of PSMB5 or control safe harbor regions were designed and synthesized on an oligonucleotide array and cloned into an sgRNA expressing vector. This vector was integrated into cells expressing dCas9 and MS2-AIDΔ to generate mutations. Cells were pulsed with bortezomib, after which the PSMB5 exonic loci were sequenced. Plots of the enrichment of mutation at each base position are shown for the PSMB5 locus in both PSMB5 and safe harbor targeted libraries for one biological replicate.

FIG. 11 shows plots of the enrichment of mutations for individual PSMB5 exons in the experiments described above for FIG. 10. Positions that were above 20-fold enriched (black dashed line) in both replicates were identified as possible candidates.

FIG. 12 is a bar plot showing the density of live cells having a PSMB5 mutation after selection with bortezomib. Mutations were installed into K562 cells and selected with bortezomib. Error bars indicate standard error.

FIG. 13 shows data from experiments testing the knock-in and validation of novel bortezomib-resistant PSMB5 variants. Bortezomib resistant mutations observed in PSMB5 (FIG. 10-12) were knocked-in to K562 cells and populations were selected with bortezomib. The corresponding PSMB5 exons for the five most viable mutations were amplified, cloned into pCR-Blunt, and sequenced individually. Results for three replicates are shown in the table for 5 mutations. The sequences of individual colonies with mutations or insertions/deletions are shown; the targeted base is in bold.

FIG. 14 shows improved mutagenesis using AID*Δ. sgRNAs targeting either GFP (sgGFP.3 and sgGFP.10) or a safe harbor locus (sgSafe.2) were integrated into cells expressing dCas9, MS2-AID*Δ, GFP, and mCherry. The GFP and mCherry loci were sequenced. Enrichment of mutation at each base position is shown for three replicates of the experiment. The average number of mutations per sequence was calculated and are provided below in Table 8.

FIG. 15 shows data from experiments testing the enhanced mutagenesis of genes, promoters, and multiple loci with hyperactive AID*Δ. sgGFP.3, sgGFP.10, and sgSafe.2 were infected into cells expressing dCas9, MS2-733 AID*Δ, GFP, and mCherry. The GFP and mCherry loci were sequenced. Enrichment of mutations at positions relative to the sgRNA PAM is shown for 2 GFP-targeting sgRNAs, sgGFP.3 and sgGFP.10, using either AIDΔ (top plot) or hyperactive AID*Δ(bottom plot). The shaded rectangles highlight the respective hotspot regions. (right)

FIG. 16 is a bar plot showing the frequencies of mutated sequences in the respective hotspots identified in the experiment described for FIG. 15 above.

FIG. 17 shows data collected from experiments in which sgRNAs were designed to target six endogenous loci. Gene diagrams for each locus are shown indicating the position of the respective guides. Cells expressing dCas9 and MS2-AID*Δ were infected with the sgRNAs, and the loci were sequenced. The plots show the enrichment of mutations at positions relative to the PAM at each of the loci. Some samples with sgRNAs targeting upstream of the transcription start site were tested (grey points).

FIG. 18 shows data collected from experiments testing the simultaneous mutation of two loci. sgGFP.10 and sgmCherry.1 were integrated either individually or in combination into cells expressing dCas9, MS2-AID*Δ, GFP, and mCherry. The GFP and mCherry fluorescence were measured by flow cytometry. The percentage of GFP negative or mCherry negative cells are shown in the top panel. The bottom panel is a plot displaying the percentage of cells that have neither GFP nor mCherry. Error bars indicate standard error.

FIG. 19 is a bar plot showing the mutation frequency provided by recruitment to a target site by MS2 (approximately 0.23, left bar) and the mutation frequency provided by recruitment to a target site by a fusion comprising a hyperactive AID and dCas9 (approximately 0.58; left bar).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology related to producing mutagenic diversity at specific genomic targets, e.g., for use in the directed evolution of biomolecules such as nucleic acids and proteins. In particular embodiments, a hyperactive AID (e.g., producing more mutated nucleotides than wild-type AID) targeted with dCas9 is used to generate localized diversity within a genome (e.g., a mammalian genome, e.g., a human genome) or other target nucleic acid with minimized (e.g., insignificant, undetectable) off-target effects. The subsequent mutagenized populations produced by the AID-dCas9 provide a mutant pool for selection and directed evolution of new protein function. This system can simultaneously mutagenize multiple genomic loci, and preserves reading frame by avoiding insertions/deletions observed with native, active Cas9 used in extant technologies. While the activity of AID in antibody maturation has been shown to require transcription (12), experiments conducted during the development of the technology described herein produced mutations above background for sgRNAs targeting both upstream and downstream of the transcription start site (TSS), indicating that the present technology functions independently from transcription. Although regions upstream of the TSS may be transcribed at lower levels, these findings indicated that use of the technology is not bound to regions downstream of annotated transcription start sites and thus allows for the engineering and investigation of promoters, enhancers, and other regulatory elements.

Several directed evolution experiments were conducted during the development of the technology to illustrate this function. First, experiments were conducted and data were collected indicating that GFP is readily evolved to EGFP with the simple addition of an appropriately designed sgRNA. In addition, experiments were conducted and data were collected indicating that mutagenesis of the target of the chemotherapeutic bortezomib (PSMB5) revealed both known and novel mechanisms of resistance to bortezomib (Table 7). In particular, directed evolution of PSMB5 using the technology produced the canonical A108V/T mutation, which was identified in bortezomib resistant cell lines (38, 40) and observed in colorectal cancer patient samples (41), along with many other mutations that are consistent with the disruption of the binding pocket of bortezomib. Interestingly, the technology also produced a mutation located in exon 4 (G242D), which had not been previously connected to bortezomib resistance, and is located on the side of the protein opposite the bortezomib pocket. This indicates additional mechanisms of resistance, and may inform study of PSMB5 function as well as future drug design. Additionally, synonymous and intronic mutations were identified which require further study.

Recent work has shown that deaminases efficiently convert cytidines to thymidines as a method of correcting individual base changes (24). Experiments were conducted during the development of embodiments of the present technology using a hyperactive AID variant to create dense point mutations within a region of 100 bp surrounding an sgRNA. As in antibody somatic hypermutation, a large variety of transitions and transversions of CG bases were observed, and a low level of all base transitions was observed, which can be enriched by selection.

The present technology presents a number of significant advantages over existing methods used to engineer proteins. First, the specific targeting of AID allows continuous mutagenesis and evolution of protein function as is observed in antibody affinity maturation, as opposed to using a synthetic library of defined size. Previous efforts to use AID for mutagenesis used overexpression of both AID and the target protein. In those studies, the target was present at non-physiological levels, and cells had significant genome instability and potentially confounding off-target mutations due to promiscuous AID activity (42, 43). While advances have been made to understand the targeting of somatic hypermutation to the Ig locus (10,44), the known control elements are difficult to install systematically throughout the genome. The present technology overcomes both of these limitations by using dCas9 to target somatic hypermutation, which should facilitate both engineering of new biomolecules as well as provide a research tool to study the SHM process itself. Repeated rounds of mutagenesis using the present technology allow exploration of a virtually limitless sequence space, since combinations of mutations observed with single sgRNAs can be multiplied by simultaneously targeting multiple genomic locations. This system makes it possible to study the co-evolution of two or more interacting proteins expressed at endogenous levels, and provides a streamlined strategy for selection of enhanced antibody and enzyme function via mutagenesis in a native context.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

“Peptide nucleic acid” means a DNA mimic that incorporates a peptide-like polyamide backbone.

As used herein, the term “% sequence identity” refers to the percentage of nucleotides or nucleotide analogs in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including blastn, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

“Mismatch” means a nucleobase of a first nucleic acid that is not capable of pairing with a nucleobase at a corresponding position of a second nucleic acid.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see e.g., Anderson and Young, Quantitative Filter Hybridization, in Nucleic Acid Hybridization (1985). Other references (e.g., Allawi and SantaLucia, Biochemistry 36: 10581-94 (1997) include more sophisticated computations which account for structural, environmental, and sequence characteristics to calculate Tₘ. For example, in some embodiments these computations provide an improved estimate of Tₘ for short nucleic acid probes and targets.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded”. In some embodiments, any base-paired nucleic acid is a “double-stranded nucleic acid”

The term “gene” refers to a DNA sequence that comprises control and coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or function is retained.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprising two or more deoxyribonucleotides or ribonucleotides, preferably at least 5 nucleotides, more preferably at least about 10 to 15 nucleotides and more preferably at least about 15 to 30 nucleotides. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

As used herein, the terms “subject” and “patient” refer to any organisms including plants, microorganisms, and animals (e.g., mammals such as dogs, cats, livestock, and humans).

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

The term “label” as used herein refers to any atom or molecule that can be used to provide a detectable (preferably quantifiable) effect, and that can be attached to a nucleic acid or protein. Labels include, but are not limited to, dyes (e.g., fluorescent dyes or moieties); radiolabels such as ³²P; binding moieties such as biotin; haptens such as digoxgenin; luminogenic, phosphorescent, or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein, “moiety” refers to one of two or more parts into which something may be divided, such as, for example, the various parts of an oligonucleotide, a molecule, a chemical group, a domain, a probe, etc.

The terms “protein” and “polypeptide” refer to compounds comprising amino acids joined via peptide bonds and are used interchangeably. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine Val, V. As used herein, the codes Xaa and X refer to any amino acid.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. Codes for degenerate positions in a nucleotide sequence are: R (G or A), Y (T/U or C), M (A or C), K (G or T/U), S (G or C), W (A or T/U), B (G or C or T/U), D (A or G or T/U), H (A or C or T/U), V (A or G or C), or N (A or G or C or T/U), gap (-).

As used herein, the term “deaminase” refers to an enzyme that catalyzes a deamination reaction. In some embodiments, the deaminase is a cytidine deaminase, catalyzing the hydrolytic deamination of cytidine or deoxycytidine to uracil or deoxyuracil, respectively.

As used herein, the term “effective amount” refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a recombinase may refer to the amount of the recombinase that is sufficient to induce recombination at a target site specifically bound and recombined by the recombinase. As will be appreciated by the skilled artisan, the effective amount of an agent, e.g., a nuclease, a recombinase, a hybrid protein, a fusion protein, a protein dimer, a complex of a protein (or protein dimer) and a polynucleotide, or a polynucleotide, may vary depending on various factors as, for example, on the desired biological response, the specific allele, genome, target site, cell, or tissue being targeted, and the agent being used.

As used herein, the term “linker” refers to a chemical group or a molecule linking two molecules or moieties. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

As used herein, the term “mutation” refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The term “target site” refers to a sequence within a nucleic acid molecule that is deaminated by a deaminase or a fusion protein comprising a deaminase, (e.g., a dCas9-deaminase fusion protein provided herein).

### DESCRIPTION

Extant technologies related to the engineering and study of protein function by directed evolution utilizes DNA libraries having a defined size or using non-specific, global mutagenesis methods. Provided herein is a technology that modifies the components and processes of somatic hypermutation involved in, for example, antibody affinity maturation to provide a technology for in situ protein engineering. In particular, some embodiments of the technology provided herein comprise use of a catalytically inactive Cas9 (dCas9) and variants of a deaminase (e.g., activation-induced cytidine deaminase (AID)). In some embodiments, the technology provides methods for specific mutagenesis of endogenous targets with limited (e.g., minimized, reduced, insignificant, and/or undectable) off-target mutagenesis. In some embodiments, the technology produces diverse libraries of localized point mutations and the technology finds use to mutagenize multiple genomic locations simultaneously. This technology is an improvement over extant technologies that produce insertions and deletions, e.g., technologies comprising use of an active Cas9.

During the development of embodiments of this technology, experiments were conducted to test the specific mutagenesis of defined targets. For example, experiments were conducted in which the technology was used to mutagenize green fluorescent protein (GFP) to provide a pool of mutant GFP proteins that were tested for spectral shifts relative to the wild-type GFP protein. Data collected during analysis of the mutant GFP proteins identified spectrum-shifted variants, included enhanced GFP (EGFP).

In addition, experiments were conducted during the development of embodiments of the technology in which mutations were introduced into the gene encoding a target of the cancer therapeutic bortezomib (proteasome subunit beta type-5 (PSMB5)), and both known and novel mutations were identified in the PSMB5 mutant pool that confer resistance to treatment.

Finally, during the development of embodiments of the technology provided herein, a hyperactive AID variant was produced and tested. Data collected indicated that the mutant AID has an increased mutagenesis activity relative to the wild-type AID. Further, data collected during the experiments indicated that the mutant AID mutagenized endogenous loci both upstream and downstream of transcriptional start sites. In sum, the data collected from experiments conducted during the development of the technology indicated that the technology finds use in producing highly complex libraries of genetic variants in a native biological context, which can be broadly applied to investigate and improve protein and/or nucleic acid function. Applications include, but are not limited to, directed evolution (e.g., protein, peptide, nucleic acid), generation of antibodies and enzymes, co-evolution of protein surfaces, engineering of binding site specificities, mutagenesis and selections systems, methods, and kits, multiplex mutagenesis of several sites within a target (e.g., a genome) at once, and increased diversity of mutations in mutagenesis applications compared to available technique (e.g., rather than conversion of just C to T or G to A, provided herein is the ability to convert to any base). Although the disclosure herein refers to certain illustrated embodiments, it is to be understood that these embodiments are presented by way of example and not by way of limitation.

### Nucleic Acid Editing Enzymes

Embodiments comprise use of a nucleic acid editing enzyme. For example, some embodiments comprise use of an enzyme from the apolipoprotein B mRNA-editing complex (APOBEC) family of cytosine deaminase enzymes, which encompasses eleven proteins that serve to initiate mutagenesis in a controlled and beneficial manner.

Particular embodiments comprise use of the APOBEC family member known as activation-induced cytidine deaminase (known variously as, e.g., AICDA, AID, ARP2, CDA2, HIGM2, and HEL-S-284; UniProt accession Q9GZX7; NCBI RefSeq (mRNA) accession NM_020661 and NCBI RefSeq (protein) accession NP_065712.1) is a 24-kDa enzyme encoded in humans by the AICDA gene (located on human chromosome 12 and at positions 8,602,166 to 8,612,888). The AID protein is involved in producing antibody diversity in B cells of the immune system, e.g., by the processes of somatic hypermutation, gene conversion, and class-switch recombination of immunoglobulin genes.

AID is a DNA-editing deaminase that is a member of the cytidine deaminase family. In particular, the AID protein creates mutations in DNA by deamination of cytosine, which converts the cytosine base to a uracil base. That is, the AID protein changes a C:G base pair into a U:G mismatch. Then, during DNA replication, the replication enzymes recognize the uracil as a thymidine, thus resulting in the conversion of the C:G base pair to a TA base pair. AID is also known to generate other types of mutations (e.g., C:G to A:T), e.g., during B lymphocyte somatic hypermutation processes. While the mechanism by which these other types of mutations are created is not completely understood, an understanding of the mechanism is not required to practice the technology provided herein.

AID activity in B cells is controlled by modulating AID expression. AID is induced by transcription factors, e.g., E47, HoxC4, Irf8 and Pax5; AID is inhibited by other factors, e.g., Blimp1 and Id2. At the post-transcriptional level of regulation, AID expression is silenced by mir-155, a small non-coding microRNA controlled by IL-10 cytokine B cell signaling.

Some embodiments comprise use of an enzyme from the apolipoprotein B mRNA-editing complex (APOBEC) family of cytosine deaminase enzymes, which encompasses eleven proteins that serve to initiate mutagenesis in a controlled and beneficial manner.

In some embodiments, the nucleic acid editing enzyme is an adenosine deaminase. For example, some embodiments comprise use of an ADAT family adenosine deaminase as a replacement for an AID enzyme as the technology is described for use of an AID enzyme (e.g., an adenosine deaminase is fused to an MS2 protein).

dCas9

The technology comprises use of a sequence-specific nucleic acid binding component (e.g., molecule, biomolecule, or complex of one or more molecules and/or biomolecules) to target specific genetic loci for mutagenesis. In exemplary embodiments, the sequence-specific nucleic acid binding component comprises an enzymatically inactive, or “dead”, Cas9 protein (“dCas9”) and a guide RNA (“gRNA”). While nucleic acid-binding molecules such as the clustered regularly interspaced short palindromic repeats (CRISPR) and CRISPR-associated proteins (Cas) (CRISPR/Cas) system have been used extensively for genome editing in cells of various types and species, recombinant and engineered nucleic acid-binding proteins find use in the present technology to provide sequence specificity.

The Cas9 protein was discovered as a component of the bacterial adaptive immune system (see, e.g., Barrangou et al. (2007) “CRISPR provides acquired resistance against viruses in prokaryotes” Science 315: 1709-1712). Cas9 is an RNA-guided endonuclease that targets and destroys foreign DNA in bacteria using RNA:DNA base-pairing between the gRNA and foreign DNA to provide sequence specificity. Recently, Cas9/gRNA complexes have found use in genome editing (see, e.g., Doudna et al. (2014) “The new frontier of genome engineering with CRISPR-Cas9” Science 346: 6213).

Accordingly, some Cas9/RNA complexes comprise two RNA molecules: (1) a CRISPR RNA (crRNA), possessing a nucleotide sequence complementary to the target nucleotide sequence; and (2) a trans-activating crRNA (tracrRNA). In this mode, Cas9 functions as an RNA-guided nuclease that uses both the crRNA and tracrRNA to recognize and cleave a target sequence. Recently, a single chimeric guide RNA (sgRNA) mimicking the structure of the annealed crRNA/tracrRNA has become more widely used than crRNA/tracrRNA because the gRNA approach provides a simplified system with only two components (e.g., the Cas9 and the sgRNA). Thus, sequence-specific binding to a nucleic acid can be guided by a natural dual-RNA complex (e.g., comprising a crRNA, a tracrRNA, and Cas9) or a chimeric single-guide RNA (e.g., a sgRNA and Cas9). (see, e.g., Jinek et al. (2012) “A Programmable Dual-RNA-Guided DNA Endonuclease in Adaptive Bacterial Immunity” Science 337:816-821).

As used herein, the targeting region of a crRNA (2-RNA system) or a sgRNA (single guide system) is referred to as the “guide RNA” (gRNA). In some embodiments, the gRNA comprises, consists of, or essentially consists of 10 to 50 bases, e.g., 15 to 40 bases, e.g., 15 to 30 bases, e.g., 15 to 25 bases (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases). Methods are known in the art for determining the length of the gRNA that provides the most efficient target recognition for a Cas9. See, e.g., Lee et al. (2016) “The Neisseria meningitidis CRISPR-Cas9 System Enables Specific Genome Editing in Mammalian Cells” Mol Ther 24(3): 645-54.

Accordingly, in some embodiments the gRNA is a short synthetic RNA comprising a “scaffold” sequence for Cas9-binding and a user-defined approximately 20-nucleotide “targeting” sequence that is complementary to the nucleic acid target (e.g., complementary to the target site). In some embodiments, the gRNA further comprises a “binding” sequence that specifically interacts with another biomolecule, e.g., a sequence that forms a secondary structure specifically bound by an MS2 protein.

In some embodiments, DNA targeting specificity is determined by two factors: 1) a DNA sequence matching the gRNA targeting sequence and a protospacer adjacent motif (PAM) directly downstream of the target sequence. Some Cas9/gRNA complexes recognize a DNA sequence comprising a protospacer adjacent motif (PAM) sequence and the adjacent approximately 20 bases complementary to the gRNA. Canonical PAM sequences are NGG or NAG for Cas9 from Streptococcus pyogenes and NNNNGATT for the Cas9 from Neisseria meningitidis. Following DNA recognition by hybridization of the gRNA to the DNA target sequence, native Cas9 cleaves the DNA sequence via an intrinsic nuclease activity. For genome editing and other purposes, the CRISPR/Cas system from S. pyogenes has been used most often. Using this system, one can target a given target nucleic acid (e.g., for editing or other manipulation) by designing a gRNA having nucleotide sequence complementary to an approximately 20-base DNA sequence 5′-adjacent to the PAM. Methods are known in the art for determining the PAM sequence that provides the most efficient target recognition for a Cas9. See, e.g., Zhang et al. (2013) “Processing-independent CRISPR RNAs limit natural transformation in Neisseria meningitidis” Molecular Cell 50: 488-503; Lee et al., supra.

In contrast to extant genome editing technologies in which the Cas9 protein cleaves a nucleic acid, the present technology comprises use of a catalytically inactive form of Cas9 (“dead Cas9” or “dCas9”), in which point mutations are introduced that disable the nuclease activity. In some embodiments, the dCas9 protein is from S. pyogenes. In some embodiments, the dCas9 protein comprises mutations at, e.g., D10, E762, H983, and/or D986; and at H840 and/or N863, e.g., at D10 and H840, e.g., D10A or DION and H840A or H840N or H840Y. In some embodiments, the dCas9 is provided as a fusion protein comprising a functional domain for attaching the dCas9 to a solid surface (e.g., an epitope tag, linker peptide, etc.).

For example, in some embodiments, the dCas9 protein has less than 50%, less than 40%, less than 30%, less than 20%, less than 10%, less than 5%, or less than 1% of the nuclease activity of the corresponding wild-type Cas9 polypeptide. In some embodiments, the modified form of the Cas9/Csn1 polypeptide has no substantial nuclease activity (e.g., insignificant and/or undetectable nuclease activity).

The dCas9/gRNA complex binds to a target nucleic acid with a sequence specificity provided by the gRNA, but does not cleave the nucleic acid (see, e.g., Qi et al. (2013) “Repurposing CRISPR as an RNA-guided platform for sequence-specific control of gene expression” Cell 152(5): 1173-83). In this form, the dCas9/gRNA provides sequence specificity for the mutagenic technology provided herein.

Furthermore, while the Cas9/gRNA system and dCas9/gRNA system initially targeted sequences adjacent to a PAM, the dCas9/gRNA system as used herein has been engineered to target any nucleotide sequence for binding. Also, Cas9 and dCas9 orthologs encoded by compact genes (e.g., Cas9 from Staphylococcus aureus) are known (see, e.g., Ran et al. (2015) “In vivo genome editing using Staphylococcus aureus Cas9” Nature 520: 186-191), which improves the cloning and manipulation of the Cas9 components in vitro.

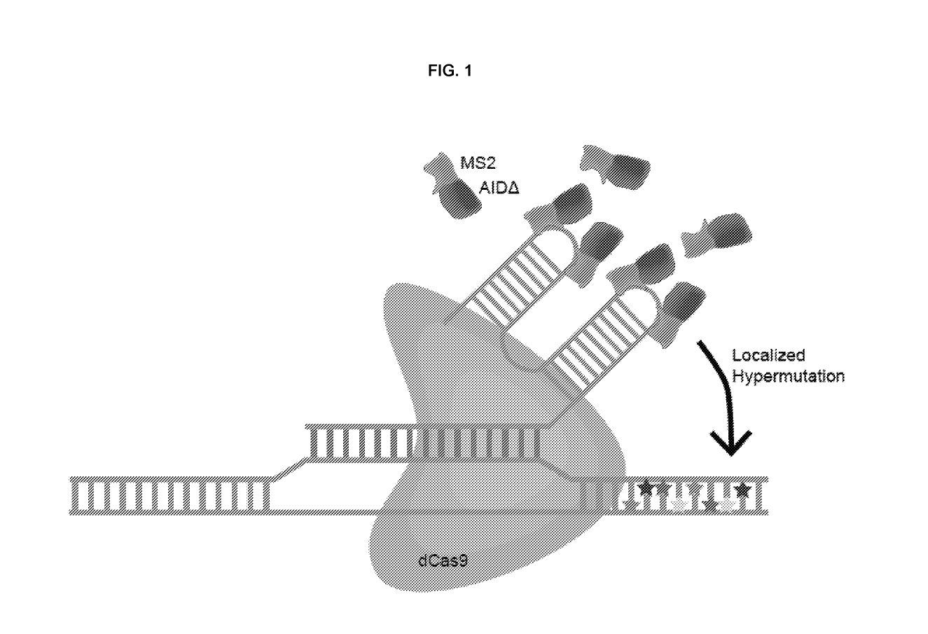

A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013), and a number of Cas9 proteins are listed in supplementary FIG. 1 and supplementary table 1 thereof, which are incorporated by reference herein. Additional Cas9 proteins are described in Esvelt et al., Nat Methods. 2013 November; 10(11)1116-21 and Fonfara et al. (2014) “Phylogeny of Cas9 determines functional exchangeability of dual-RNA and Cas9 among orthologous type II CRISPR-Cas systems.” Nucleic Acids Res. 42 (4): 2577-2590.

Cas9, and thus dCas9, molecules of a variety of species find use in the technology described herein. While the S. pyogenes and S. thermophilus Cas9 molecules are widely used, Cas9 (and dCas9) molecules of, derived from, or based on the Cas9 proteins (and dCas9 proteins) of other species listed herein find use in embodiments of the technology. Accordingly, the technology provides for the replacement of S. pyogenes and S. thermophilus Cas9 and dCas9 molecules with Cas9 and dCas9 molecules from other species, e.g:

| GenBank | |
|---|---|
| Acc No. | Bacterium |
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJ010A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp-B510 |
| 92109262 | Nitrobacter hamburgensis X14 |
| 148255343 | Bradyrhizobium sp-BTAil |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

The technology described herein encompasses the use of a dCas9 derived from any Cas9 protein (e.g., as listed above) and their corresponding guide RNAs or other guide RNAs that are compatible. The Cas9 from Streptococcus thermophilus LMD-9 CRISPR1 system has been shown to function in human cells (see, e.g., Cong et al. (2013) Science 339: 819). Additionally, Jinek showed in vitro that Cas9 orthologs from S. thermophilus and L. innocua, can be guided by a dual S. pyogenes gRNA to cleave target plasmid DNA.

In some embodiments, the present technology comprises the Cas9 protein from S. pyogenes, either as encoded in bacteria or codon-optimized for expression in mammalian cells, containing mutations at D10, E762, H983, or D986 and H840 or N863, e.g., D10A/D10N and H840A/H840N/H840Y, to render the nuclease portion of the protein catalytically inactive; substitutions at these positions are, in some embodiments, alanine (Nishimasu (2014) Cell 156: 935-949) or, in some embodiments, other residues, e.g., glutamine, asparagine, tyrosine, serine, or aspartate, e.g., E762Q, H983N, H983Y, D986N, N863D, N863S, or N863H. The sequence of one S. pyogenes dCas9 protein that finds use in the technology provided herein is described in US20160010076, which is incorporated herein by reference in its entirety.

For example, in some embodiments, the dCas9 used herein is at least about 50% identical to the amino acid sequence of S. pyogenes Cas9, e.g., at least 50%, 55%, 60%, 65%, 70%, 75%, 80%, 85%, 90%, 95%, 97%, 98%, or 99% or more identical to the following amino acid sequence of dCas9 comprising the D10A and H840A substitutions (SEQ ID NO: 1):

```
Met Asp Lys Lys Tyr Ser Ile Gly Leu Ala Ile Gly Thr Asn Ser Val
1 5 10 15
Gly Trp Ala Val Ile Thr Asp Glu Tyr Lys Val Pro Ser Lys Lys Phe
                        20 25 30
Lys Val Leu Gly Asn Thr Asp Arg His Ser Ile Lys Lys Asn Leu Ile
                35 40 45
Gly Ala Leu Leu Phe Asp Ser Gly Glu Thr Ala Glu Ala Thr Arg Leu
        50 55 60
Lys Arg Thr Ala Arg Arg Arg Tyr Thr Arg Arg Lys Asn Arg Ile Cys
65 70 75 80
Tyr Leu Gln Glu Ile Phe Ser Asn Glu Met Ala Lys Val Asp Asp Ser
                                85 90 95
Phe Phe His Arg Leu Glu Glu Ser Phe Leu Val Glu Glu Asp Lys Lys
                        100 105 110
His Glu Arg His Pro Ile Phe Gly Asn Ile Val Asp Glu Val Ala Tyr
                115 120 125
His Glu Lys Tyr Pro Thr Ile Tyr His Leu Arg Lys Lys Leu Val Asp
        130 135 140
Ser Thr Asp Lys Ala Asp Leu Arg Leu Ile Tyr Leu Ala Leu Ala His
145 150 155 160
Met Ile Lys Phe Arg Gly His Phe Leu Ile Glu Gly Asp Leu Asn Pro
                                165 170 175
Asp Asn Ser Asp Val Asp Lys Leu Phe Ile Gln Leu Val Gln Thr Tyr
                        180 185 190
Asn Gln Leu Phe Glu Glu Asn Pro Ile Asn Ala Ser Gly Val Asp Ala
                195 200 205
Lys Ala Ile Leu Ser Ala Arg Leu Ser Lys Ser Arg Arg Leu Glu Asn
        210 215 220
Leu Ile Ala Gln Leu Pro Gly Glu Lys Lys Asn Gly Leu Phe Gly Asn
225 230 235 240
Leu Ile Ala Leu Ser Leu Gly Leu Thr Pro Asn Phe Lys Ser Asn Phe
                                245 250 255
Asp Leu Ala Glu Asp Ala Lys Leu Gln Leu Ser Lys Asp Thr Tyr Asp
                        260 265 270
Asp Asp Leu Asp Asn Leu Leu Ala Gln Ile Gly Asp Gln Tyr Ala Asp
                275 280 285
Leu Phe Leu Ala Ala Lys Asn Leu Ser Asp Ala Ile Leu Leu Ser Asp
        290 295 300
Ile Leu Arg Val Asn Thr Glu Ile Thr Lys Ala Pro Leu Ser Ala Ser
305 310 315 320
Met Ile Lys Arg Tyr Asp Glu His His Gln Asp Leu Thr Leu Leu Lys
                                325 330 335
Ala Leu Val Arg Gln Gln Leu Pro Glu Lys Tyr Lys Glu Ile Phe Phe
                        340 345 350
Asp Gln Ser Lys Asn Gly Tyr Ala Gly Tyr Ile Asp Gly Gly Ala Ser
                355 360 365
Gln Glu Glu Phe Tyr Lys Phe Ile Lys Pro Ile Leu Glu Lys Met Asp
        370 375 380
Gly Thr Glu Glu Leu Leu Val Lys Leu Asn Arg Glu Asp Leu Leu Arg
385 390 395 400
Lys Gln Arg Thr Phe Asp Asn Gly Ser Ile Pro His Gln Ile His Leu
                                405 410 415
Gly Glu Leu His Ala Ile Leu Arg Arg Gln Glu Asp Phe Tyr Pro Phe
                        420 425 430
Leu Lys Asp Asn Arg Glu Lys Ile Glu Lys Ile Leu Thr Phe Arg Ile
                435 440 445
Pro Tyr Tyr Val Gly Pro Leu Ala Arg Gly Asn Ser Arg Phe Ala Trp
        450 455 460
Met Thr Arg Lys Ser Glu Glu Thr Ile Thr Pro Trp Asn Phe Glu Glu
465 470 475 480
Val Val Asp Lys Gly Ala Ser Ala Gln Ser Phe Ile Glu Arg Met Thr
                                485 490 495
Asn Phe Asp Lys Asn Leu Pro Asn Glu Lys Val Leu Pro Lys His Ser
                        500 505 510
Leu Leu Tyr Glu Tyr Phe Thr Val Tyr Asn Glu Leu Thr Lys Val Lys
                515 520 525
Tyr Val Thr Glu Gly Met Arg Lys Pro Ala Phe Leu Ser Gly Glu Gln
        530 535 540
Lys Lys Ala Ile Val Asp Leu Leu Phe Lys Thr Asn Arg Lys Val Thr
545 550 555 560
Val Lys Gln Leu Lys Glu Asp Tyr Phe Lys Lys Ile Glu Cys Phe Asp
                                565 570 575
Ser Val Glu Ile Ser Gly Val Glu Asp Arg Phe Asn Ala Ser Leu Gly
                        580 585 590
Thr Tyr His Asp Leu Leu Lys Ile Ile Lys Asp Lys Asp Phe Leu Asp
                595 600 605
Asn Glu Glu Asn Glu Asp Ile Leu Glu Asp Ile Val Leu Thr Leu Thr
        610 615 620
Leu Phe Glu Asp Arg Glu Met Ile Glu Glu Arg Leu Lys Thr Tyr Ala
625 630 635 640
His Leu Phe Asp Asp Lys Val Met Lys Gln Leu Lys Arg Arg Arg Tyr
                                645 650 655
Thr Gly Trp Gly Arg Leu Ser Arg Lys Leu Ile Asn Gly Ile Arg Asp
                        660 665 670
Lys Gln Ser Gly Lys Thr Ile Leu Asp Phe Leu Lys Ser Asp Gly Phe
                675 680 685
Ala Asn Arg Asn Phe Met Gln Leu Ile His Asp Asp Ser Leu Thr Phe
        690 695 700
Lys Glu Asp Ile Gln Lys Ala Gln Val Ser Gly Gln Gly Asp Ser Leu
705 710 715 720
His Glu His Ile Ala Asn Leu Ala Gly Ser Pro Ala Ile Lys Lys Gly
                                725 730 735
Ile Leu Gln Thr Val Lys Val Val Asp Glu Leu Val Lys Val Met Gly
                        740 745 750
Arg His Lys Pro Glu Asn Ile Val Ile Glu Met Ala Arg Glu Asn Gln
                755 760 765
Thr Thr Gln Lys Gly Gln Lys Asn Ser Arg Glu Arg Met Lys Arg Ile
        770 775 780
Glu Glu Gly Ile Lys Glu Leu Gly Ser Gln Ile Leu Lys Glu His Pro
785 790 795 800
Val Glu Asn Thr Gln Leu Gln Asn Glu Lys Leu Tyr Leu Tyr Tyr Leu
                                805 810 815
Gln Asn Gly Arg Asp Met Tyr Val Asp Gln Glu Leu Asp Ile Asn Arg
                        820 825 830
Leu Ser Asp Tyr Asp Val Asp Ala Ile Val Pro Gln Ser Phe Leu Lys
                835 840 845
Asp Asp Ser Ile Asp Asn Lys Val Leu Thr Arg Ser Asp Lys Asn Arg
        850 855 860
Gly Lys Ser Asp Asn Val Pro Ser Glu Glu Val Val Lys Lys Met Lys
865 870 875 880
Asn Tyr Trp Arg Gln Leu Leu Asn Ala Lys Leu Ile Thr Gln Arg Lys
                                885 890 895
Phe Asp Asn Leu Thr Lys Ala Glu Arg Gly Gly Leu Ser Glu Leu Asp
                        900 905 910
Lys Ala Gly Phe Ile Lys Arg Gln Leu Val Glu Thr Arg Gln Ile Thr
                915 920 925
Lys His Val Ala Gln Ile Leu Asp Ser Arg Met Asn Thr Lys Tyr Asp
        930 935 940
Glu Asn Asp Lys Leu Ile Arg Glu Val Lys Val Ile Thr Leu Lys Ser
945 950 955 960
Lys Leu Val Ser Asp Phe Arg Lys Asp Phe Gln Phe Tyr Lys Val Arg
                                965 970 975
Glu Ile Asn Asn Tyr His His Ala His Asp Ala Tyr Leu Asn Ala Val
                        980 985 990
Val Gly Thr Ala Leu Ile Lys Lys Tyr Pro Lys Leu Glu Ser Glu Phe
                995 1000 1005
Val Tyr Gly Asp Tyr Lys Val Tyr Asp Val Arg Lys Met Ile Ala
        1010 1015 1020
Lys Ser Glu Gln Glu Ile Gly Lys Ala Thr Ala Lys Tyr Phe Phe
        1025 1030 1035
Tyr Ser Asn Ile Met Asn Phe Phe Lys Thr Glu Ile Thr Leu Ala
        1040 1045 1050
Asn Gly Glu Ile Arg Lys Arg Pro Leu Ile Glu Thr Asn Gly Glu
        1055 1060 1065
Thr Gly Glu Ile Val Trp Asp Lys Gly Arg Asp Phe Ala Thr Val
        1070 1075 1080
Arg Lys Val Leu Ser Met Pro Gln Val Asn Ile Val Lys Lys Thr
        1085 1090 1095
Glu Val Gln Thr Gly Gly Phe Ser Lys Glu Ser Ile Leu Pro Lys
        1100 1105 1110
Arg Asn Ser Asp Lys Leu Ile Ala Arg Lys Lys Asp Trp Asp Pro
        1115 1120 1125
Lys Lys Tyr Gly Gly Phe Asp Ser Pro Thr Val Ala Tyr Ser Val
        1130 1135 1140
Leu Val Val Ala Lys Val Glu Lys Gly Lys Ser Lys Lys Leu Lys
        1145 1150 1155
Ser Val Lys Glu Leu Leu Gly Ile Thr Ile Met Glu Arg Ser Ser
        1160 1165 1170
Phe Glu Lys Asn Pro Ile Asp Phe Leu Glu Ala Lys Gly Tyr Lys
        1175 1180 1185
Glu Val Lys Lys Asp Leu Ile Ile Lys Leu Pro Lys Tyr Ser Leu
        1190 1195 1200
Phe Glu Leu Glu Asn Gly Arg Lys Arg Met Leu Ala Ser Ala Gly
        1205 1210 1215
Glu Leu Gln Lys Gly Asn Glu Leu Ala Leu Pro Ser Lys Tyr Val
        1220 1225 1230
Asn Phe Leu Tyr Leu Ala Ser His Tyr Glu Lys Leu Lys Gly Ser
        1235 1240 1245
Pro Glu Asp Asn Glu Gln Lys Gln Leu Phe Val Glu Gln His Lys
        1250 1255 1260
His Tyr Leu Asp Glu Ile Ile Glu Gln Ile Ser Glu Phe Ser Lys
        1265 1270 1275
Arg Val Ile Leu Ala Asp Ala Asn Leu Asp Lys Val Leu Ser Ala
        1280 1285 1290
Tyr Asn Lys His Arg Asp Lys Pro Ile Arg Glu Gln Ala Glu Asn
        1295 1300 1305
Ile Ile His Leu Phe Thr Leu Thr Asn Leu Gly Ala Pro Ala Ala
        1310 1315 1320
Phe Lys Tyr Phe Asp Thr Thr Ile Asp Arg Lys Arg Tyr Thr Ser
        1325 1330 1335
Thr Lys Glu Val Leu Asp Ala Thr Leu Ile His Gln Ser Ile Thr
        1340 1345 1350
Gly Leu Tyr Glu Thr Arg Ile Asp Leu Ser Gln Leu Gly Gly Asp
        1355 1360 1365
```

In some embodiments, the technology comprises use of a nucleotide sequence that is approximately 50%, 55%, 60%, 65%, 70%, 75%, 80%, 85%, 90%, 95%, 99% or 100% identical to a nucleotide sequence that encodes a protein described by SEQ ID NO: 1.

In some embodiments, the dCas9 used herein is at least about 50% identical to the sequence of the catalytically inactive S. pyogenes Cas9, i.e., at least 50%, 55%, 60%, 65%, 70%, 75%, 80%, 85%, 90%, 95%, 99% or 100% identical to SEQ ID NO: 1, wherein the mutations at D10 and H840, e.g., D10A/D10N and H840A/H840N/H840Y are maintained.

In some embodiments, any differences from SEQ ID NO: 1 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11)1116-21 and Fonfara et al., Nucl. Acids Res. (2014) 42 (4): 2577-2590. [Epub ahead of print 2013 Nov. 22] doi:10.1093/nar/gkt1074, and wherein the mutations at D10 and H840, e.g., D10A/D10N and H840A/H840N/H840Y are maintained.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence) is aligned. The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For purposes of the present application, the percent identity between two amino acid sequences is determined using the Needleman and Wunsch ((1970) J. Mol. Biol. 48:444-453) algorithm which has been incorporated into the GAP program in the GCG software package, using a Blosum 62 scoring matrix with a gap penalty of 12, a gap extend penalty of 4, and a frameshift gap penalty of 5.

Accordingly, as used herein the term “Cas9” refers to an RNA-guided nuclease comprising a Cas9 protein, or a fragment thereof (e.g., a protein comprising an active or inactive DNA cleavage domain of Cas9 (a “dCas9”), and/or the gRNA binding domain of Cas9). Suitable Cas9 and/or dCas9 nucleases and sequences will be apparent to those of skill in the art based on this disclosure, and such Cas9 and/or dCas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### Bacteriophage MS2 RNA and MS2 Protein

MS2 bacteriophage coat protein interacts specifically with a stem-loop structure from the MS2 phage genome to form an RNA-protein complex (Johansson et al (1997) “RNA Recognition by the MS2 Phage Coat Protein” Seminars in VIROLOGY 8: 176). The nucleotide sequence promoting binding of the MS2 protein to a nucleic acid is a hairpin comprising the Shine-Dalgarno sequence and the initiation codon of the replicase gene (e.g., AAACAUGAGGAUUACCCAUGUCG (SEQ ID NO: 843)). However, experiments have indicated that tight binding of MS2 to the MS2 nucleic acid is not solely sequence-specific, but is mediated by a combination of sequence and specific structure elements. In particular, MS2 coat protein binds to a nucleic acid comprising four specific single-stranded residues held in place by a characteristic secondary structure of the MS2 stem-loop (Romaniuk et al (1987) “RNA binding site of R17 coat protein” Biochemistry 26: 1563-1568; Schneider et al (1992) “Selection of high affinity RNA ligands to the bacteriophage R17 coat protein” J. Mol. Biol. 288: 862-869). In some embodiments, the stem loop has a primary structure of:

(SEQ ID NO: 844)
N₁N₂N₃N₄ - A - N₅N₆ - AN₇YA - N₆, N₅, -
N₄, N₃, N₂, N₁,,

wherein N denotes any nucleotide, Y denotes a pyrimidine (e.g., T or C), and subscripted nucleotides are complementary to their primed counterparts (e.g., N₁ is complementary to N₁, N₂ is complementary to N_{2′}, etc.) to form the duplex stem of the structure. AN₇YA forms the loop and the A in the fifth nucleotide position is an unmatched, bulged nucleotide.

In some embodiments, the technology comprises use of an MS2 coat protein comprising an amino acid sequence of:

(SEQ ID NO: 845)
MASNFTQFVLVDNGGTGDVTVAPSNFANGVAEWISSNSRSQAYKVTCSVR
QSSAQNRKYTIKVEVPKVATQTVGGVELPVAAWRSYLNMELTIPIFATNS
DCELIVKAMQGLLKDGNPIPSAIAANSGIY

In some embodiments, the technology comprises use of an MS2 coat protein comprising an amino acid sequence that is at least about 50% identical to the amino acid sequence of SEQ ID NO: 845, e.g., at least 50%, 55%, 60%, 65%, 70%, 75%, 80%, 85%, 90%, 95%, 97%, 98%, or 99% identical to SEQ ID NO: 845. In some embodiments, the technology comprises use of an MS2 coat protein comprising an amino acid sequence that is a subsequence of SEQ ID NO: 845 that is at least about 50% of the length of the amino acid sequence of SEQ ID NO: 845, e.g., at least 50%, 55%, 60%, 65%, 70%, 75%, 80%, 85%, 90%, 95%, 97%, 98%, or 99% as long as the length of SEQ ID NO: 845. In some embodiments, the coat protein comprises the sequence of SEQ ID NO: 845 without the first methionine, e.g., a protein comprising a sequence provided by:

(SEQ ID NO: 846)
ASNFTQFVLVDNGGTGDVTVAPSNFANGVAEWISSNSRSQAYKVTCSVRQ
SSAQNRKYTIKVEVPKVATQTVGGVELPVAAWRSYLNMELTIPIFATNSD
CELIVKAMQGLLKDGNPIPSAIAANSGIY

In some embodiments, the technology comprises use of an MS2 coat protein comprising an amino acid sequence that is at least about 50% identical to the amino acid sequence of SEQ ID NO: 846, e.g., at least 50%, 55%, 60%, 65%, 70%, 75%, 80%, 85%, 90%, 95%, 97%, 98%, or 99% identical to SEQ ID NO: 846. In some embodiments, the technology comprises use of an MS2 coat protein comprising an amino acid sequence that is a subsequence of SEQ ID NO: 846 that is at least about 50% of the length of the the amino acid sequence of SEQ ID NO: 846, e.g., at least 50%, 55%, 60%, 65%, 70%, 75%, 80%, 85%, 90%, 95%, 97%, 98%, or 99% as long as the length of SEQ ID NO: 846.

The nucleotide sequence of the gene encoding the MS2 coat protein is known (see, e.g., Nature 237: 82-88(1972)). Further, amino acid substitutions that are deleterious for RNA stem-loop binding are known (Peabody, EMBO J 12: 595, 1993). Thus, variants of SEQ ID NO: 845 that retain stem-loop binding are provided herein, e.g., variants of SEQ ID NO: 845 or 846 that have substitutions relative to the wild-type but that do not include known substitutions that negatively affect stem-loop binding.

RNA binding by MS2 coat protein is very specific and is not disrupted other RNAs in the presence of the RNA hairpin. Thus, nucleic acids (e.g., RNA, DNA) comprising the MS2 RNA hairpin (e.g., a structure provided by SEQ ID NO: 844 or a variant thereof) specifically bind to proteins comprising the MS2 coat protein or variants of the MS2 coat protein that retain the capability to bind the MS2 stem-loop structure specifically.

While embodiments of the technology are exemplified with MS2 coat protein, it should be understood that other RNA binding proteins and associated RNAs may be employed, including but not limited to PP7 coat protein (see e.g., Lim and Peabody, Nucleic Acids Res., 30(19): 4138-4144 (2002), herein incorporated by reference in its entirety).

dCas9-Targeted Deaminase

Some aspects of the technology provide herein relate to protein-RNA complexes that comprise a RNA-guided component (e.g., a dCas9) that recruits a DNA-editing protein (e.g., an AID) to a target site, e.g., to create mutations at or near the target site (e.g., within 1 to 10, e.g., within 10 to 100 (e.g., within 10, 15, 20, 25, 30, 35, 40, 45, 50, 55, 60, 65, 70, 75, 80, 85, 90, 95, or 100) bases of the target site). The RNA-guided component comprises an RNA-binding domain that binds to a guide RNA (also referred to as gRNA or sgRNA), which, in turn, binds a target nucleic acid sequence via strand hybridization. In some embodiments, the DNA-editing protein is a deaminase that deaminates a nucleobase, such as, for example, cytidine. The deamination of a nucleobase by a deaminase leads to a point mutation at the respective residue (e.g., nucleic acid editing). Protein-RNA complexes comprising a Cas9 variant or domain (e.g., a dCas9) and a DNA editing domain can thus be used for the targeted mutagenesis of nucleic acid sequences. Such protein-RNA complexes are useful for the generation of mutant nucleic acids, mutant proteins, mutant cells, or mutant organisms to provide materials for directed evolution. Typically, the Cas9 domain does not have any nuclease activity but instead is a Cas9 fragment or a dCas9 protein or domain.

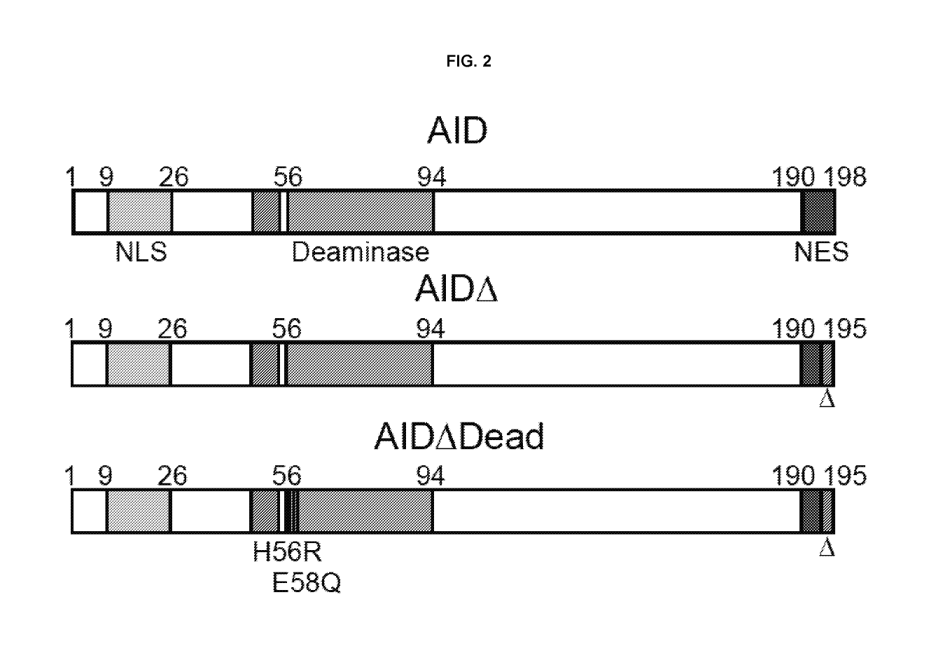

Accordingly, particular embodiments relate to a dCas9-targeted deaminase. For example, in some embodiments the technology provides a dCas9 and guide RNA (e.g., an sgRNA) that provide sequence specificity to embodiments of the technology. In some embodiments, the sgRNA comprises one or more MS2-binding hairpins. Accordingly, some embodiments provide a dCas9 bound to an sgRNA, wherein the sgRNA comprises one or more MS2-binding hairpins. Furthermore, the technology comprises one or more MS2 proteins that specifically bind to the one or more MS2-binding hairpins. In exemplary embodiments, the MS2 proteins are fused to a deaminase (e.g., an AID, e.g., an AID lacking a NES (e.g., AIDΔ), e.g., an AID lacking a NES and comprising enhanced mutagenic activity (e.g., a hyperactive AID such as AID*Δ)) (FIG. 1 and FIG. 2). The technology is not limited to these particular components or arrangements of components. For example, embodiments are contemplated in which a dCas9/sgRNA recruits a deaminase (e.g., an AID, e.g., an AID lacking a NES (e.g., AIDΔ), e.g., an AID lacking a NES and comprising enhanced mutagenic activity (e.g., a hyperactive AID such as AID*Δ)) to a particular sequence by other mechanisms. In exemplary embodiments, the dCas9 and deaminase (e.g., an AID, e.g., an AID lacking a NES (e.g., AIDΔ), e.g., an AID lacking a NES and comprising enhanced mutagenic activity (e.g., a hyperactive AID such as AID*Δ)) are expressed as a fusion protein or linked by a chemical linker (Example 8; FIG. 19). The technology also contemplates other enzymes (e.g., other deaminases) that have mutagenic capability.

As described herein, the technology provides for the creation of numerous targeted mutations. Accordingly, the technology is distinct from other technologies comprising use of a RNA-guided nuclease (or a nuclease-inactive variant thereof) that recruits a DNA-editing protein to a specific genetic locus to correct genetic defects in cells. The technology is further described in the following examples.

### EXAMPLES

### Example 1—Materials and Methods

dCas9-Targeted Deaminase Constructs and Fluorescent Protein Plasmids

The plasmids and primers used are listed in Tables 1-5.

TABLE 1
Plasmids

| Name | Description |
|---|---|
| pGH125 | dCas9-Blast |
| pGH153 | MS2-AIDΔ-Hygro |
| pGH156 | MS2-AID-Hygro |
| pGH183 | MS2-AIDΔDead-Hygro |
| pGH224 | sgRNA_2xMS2_Puro |
| pGH044 | mCherry |
| pGH045 | GFP |
| pGH220 | wtGFP |
| pGH311 | wtGFP S65T |
| pGH312 | wtGFP Q80H |
| pGH314 | wtGFP S65T, Q80H |
| pGH335 | MS2-AID*Δ-Hygro |
| pGH020 | sgRNA_G418-GFP |

TABLE 2
oligonucleotides

| Vector | Name | Sequence (5′-3′) | SEQ ID NO: |
|---|---|---|---|
| dCas9 | dCas9-Blast For | AAAAAGAGGAAGGTGGCGGCCGCTGGATCCGAGGGC | 4 |
| | (oGH255) | AGAGGAAGTCTGCTAACAT | |
| | dCas9-Blast Rev | AGGTTGATTACCGATAAGCTTGATATCGAATTC | 5 |
| | (oGH256) | | |
| MS2-AID | MS2-AID For | AAGAGGAAGGTGGCGGCCGCTGGATCCATGGACAGC | 6 |
| | (oGH272) | CTCTTGATGAACCG | |
| | MS2-AID Rev | TTCCTCTGCCCTCTCCACTGCCTGTACAAAGTCCCA | 7 |
| | (oGH273) | AAGTACGAAATGCGTC | |
| | MS2-AIDΔ Rev | TTCCTCTGCCCTCTCCACTGCCTGTACAAGTACGAA | 8 |
| | (oGH274) | ATGCGTCTCGTAAGTC | |
| | AIDΔDead Mut For | GAACGGCTGCCGCGTGCAATTGCTCTTCCTCCGCTA | 9 |
| | (oGH315) | CATCTCG | |
| | AIDΔDead Mut Rev | AAGAGCAATTGCACGCGGCAGCCGTTCTTATTGCGA | 10 |
| | (oGH316) | AGATAAC | |
| | AID*Δ K10E For | AAGAGGAAGGTGGCGGCCGCTGGATCCATGGACAGC | 11 |
| | (oGH456) | CTCTTGATGAACCGGAGGGAGTTTCTTTACCAA | |
| | AID*Δ E156G For | TACTGCTGGAATACTTTTGTAGAAAACCACGGAAGA | 12 |
| | (oGH457) | ACTTTCAAAGCCTGGGAAGG | |
| | AID*Δ E156G Rev | CCTTCCCAGGCTTTGAAAGTTCTTCCGTGGTTTTCT | 13 |
| | (oGH458) | ACAAAAGTATTCCAGCAGTA | |
| | AID*Δ T82I For | GCTGCTACCGCGTCACCTGGTTCATCTCCTGGAGCC | 14 |
| | (oGH459) | CCTGCTACGAC | |
| | AID*Δ T82I Rev | GTCGTAGCAGGGGCTCCAGGAGATGAACCAGGTGAC | 15 |
| | (oGH460) | GCGGTAGCAGC | |
| Fluorescent | GFP/mCherry For | CATTTCAGGTGTCGTGAGCTAGCCCACCATGGTGAG | 16 |
| Proteins | (oGH144) | CAAGGGCGAGGAG | |
| | GFP/mCherry Rev | CTGGCTTACTAGTCGGTTCAACTCTAGATTACTTGT | 17 |
| | (oGH146) | ACAGCTCGTCCATGCCG | |
| | wtGFP Mut For | GTGACCACCTTCAGCTACGGCGTGCAGTGC | 18 |
| | (oGH363) | | |
| | wtGFP Mut Rev | GCACTGCACGCCGTAGCTGAAGGTGGTCAC | 19 |
| | (oGH364) | | |
| | wtGFP Q80H For | ACCCCGACCACATGAAGCACCACGACTTCTTCAAGT | 20 |
| | (oGH447) | CC | |
| | wtGFP Q80H Rev | GGACTTGAAGAAGTCGTGGTGCTTCATGTGGTCGGG | 21 |
| | (oGH448) | GT | |
| | wtGFP S65T For | CCTCGTGACCACCTTCACCTACGGCGTGCAGTGCT | 22 |
| | (oGH449) | | |
| | wtGFP S65T Rev | AGCACTGCACGCCGTAGGTGAAGGTGGTCACGAGG | 23 |
| | (oGH450) | | |
| Puromycin | Puro For | TTTCTTCCATTTCAGGTGTCGTGATGTACAATGACC | 24 |
| Resistance | (oGH375) | GAGTACAAGCCCACGG | |
| | Puro Rev | ATTACCGATAAGCTTGATATCGAATTCTCAGGCACC | 25 |
| | (oGH376) | GGGCTTGCGGGTCATG | |
| | Puro BsmBI For | TCCTGGCCACCGTCGGCGTATCGCCCGACC | 26 |
| | (oGH377) | | |
| | Puro BsmBI Rev | GGTCGGGCGATACGCCGACGGTGGCCAGGA | 27 |
| | (oGH378) | | |

TABLE 3
sgRNA sequences

| Name | sgRNA Sequence (5′-3′) | Genomic Position | SEQ ID NO: |
|---|---|---|---|
| sgGFP. 1 | GGCGAGGGCGATGCCACCTA | | 28 |
| sgNegCtrl | GCTCAAGAACGCCTTCCCCAGTC | | 29 |
| sgGFP.2 | GGCACGGGCAGCTTGCCGG | | 30 |
| sgGFP.3 | AAGGGCATCGACTTCAAGG | | 31 |
| sgGFP.4 | CGATGCCCTTCAGCTCGATG | | 32 |
| sgGFP.5 | CTCGTGACCACCCTGACCTA | | 33 |
| sgGFP.6 | CAAGTTCAGCGTGTCTGGCG | | 34 |
| sgGFP.7 | CAACTACAAGACCCGCGCCG | | 35 |
| sgGFP.8 | GGTGAACCGCATCGAGCTGA | | 36 |
| sgGFP.9 | CGGCCATGATATAGACGTTG | | 37 |
| sgGFP.10 | CGTCGCCGTCCAGCTCGACC | | 38 |
| sgGFP.11 | AGCACTGCACGCCGTAGGTC | | 39 |
| sgGFP.12 | TCAGCTCGATGCGGTTCACC | | 40 |
| sgwtGFP.1 | CCGGCAAGCTGCCCGTGCCC | | 41 |
| sgwtGFP.2 | GCTTCATGTGGTCGGGGTAG | | 42 |
| sgwtGFP.3 | CGTGCTGCTTCATGTGGTCG | | 43 |
| sgwtGFP.4 | GTCGTGCTGCTTCATGTGGT | | 44 |
| sgSafe.2 | TCCCCCTCAGCCGTATT | chr12: 114129110-114129129 | 45 |
| sgSafe.4 | GATTGATATTGCCTTCT | chr12: 17350231-17350250 | 46 |
| sgSafe.5 | TCTGACTCCTAATGGAG | chr12: 114127368-114127387 | 47 |
| sgSafe.6 | ATTACTTTAGAGTAAGA | chr13: 105390313-105390332 | 48 |
| sgHBG2.1 | GGTCCATGGGTAGACAACC | chr11: 5249566-5249584 | 49 |
| sgHBG2.2 | GTGAGATTGACAAGAACAGT | chr11: 5249593-5249612 | 50 |
| sgHBG2.3 | AGGTCGCTTCTCAGGATTTG | chr11: 5249633-5249652 | 51 |
| sgHBG2.4 | GAGATCATCCAGGTGCTTTG | chr11: 5249437-5249456 | 52 |
| sgHBG2.5 | GCTACTATCACAAGCCTGTG | chr11: 5249758-5249777 | 53 |
| sgGSTP1.1 | GGAGATGTATTTGCAGCGG | chr11: 67585205-67585223 | 54 |
| sgGSTP1.2 | GGACATGGTGAATGACGGCG | chr11: 67585175-67585194 | 55 |
| sgGSTP1.3 | AGCCACCTGAGGGGTAAGGG | chr11: 67585310-67585329 | 56 |
| sgGSTP1.4 | CTGCACCCTGACCCAAGAAG | chr11: 67585341-67585360 | 57 |
| sgGSTP1.5 | TGATCAGGCGCCCAGTCACG | chr11: 67585090-67585109 | 58 |
| sgFTL.1 | GCCGAGGAGAAGCGCGA | chr19: 48965833-48965849 | 59 |
| sgFTL.2 | GCGCGAGGAGCCTTGATTTG | chr19: 48965963-48965982 | 60 |
| sgFTL.3 | CTCTATTTCCAGCGGTTAAG | chr19: 48966038-48966057 | 61 |
| sgFTL.4 | TAGCGGGAGGCGAGGCCAAG | chr19: 48965721-48965740 | 62 |
| sgFTL.5 | ACGCGCCAGCCTTCTTTGTG | chr19: 48965673-48965692 | 63 |
| sgPTPRC.1 | GTTTGTTCTTAGGGTAACAG | chr1: 198639077-198639096 | 64 |
| sgPTPRC.2 | TATCCTTGTGAAGCTAGGAG | chr1: 198638504-198638523 | 65 |
| sgPTPRC.3 | TGTTCTTGGCGCTACTGATG | chr1: 198638409-198638428 | 66 |
| sgPTPRC.4 | GGCGAGTGTGTATAGATCAG | chr1: 198697174-198697193 | 67 |
| sgPTPRC.5 | TAATGCATGTTGTTAGGGAG | chr1: 198697085-198697104 | 68 |
| sgPTPRC.6 | TGGGGAGTTAGTATACTGGG | chr1: 198696623-198696642 | 69 |
| sgPTPRC.7 | ATACACACTATAGTGGACTG | chr1: 198696605-198696624 | 70 |
| sgCD274.1 | AACTCCCACAGCATTTATCC | chr9: 5447248-5447267 | 71 |
| sgCD274.2 | ATGGGAAAATGAATGGCTGA | chr9: 5448598-5448617 | 72 |
| sgCD274.3 | CACCACCAATTCCAAGAGAG | chr9: 5462979-5462998 | 73 |
| sgCD274.4 | CAATGCAGGCTGGTTCTCAG | chr9: 5462727-5462746 | 74 |
| sgCD274.5 | TTTCATAGCCGGGAAACCTG | chr9: 5463466-5463485 | 75 |
| sgCD14.1 | TCAGGGAGGGGGACCGTAAC | chr5: 140633319-140633338 | 76 |
| sgCD14.2 | GGAGGGGGACCGTAACAGGA | chr5: 140633323-140633342 | 77 |
| sgCD14.3 | ATTCAGGGACTTGGATTTGG | chr5: 140633606-140633625 | 78 |
| sgCD14.4 | CCTCATCTGTTGGCACCAAG | chr5: 140633670-140633689 | 79 |
| sgCD14.5 | AGGAGAGAGCAACGTGCAAG | chr5: 140634212-140634231 | 80 |
| sgmCherry.1 | GCGGTCTGGGTGCCCTCGTA | | 81 |

TABLE 4
genomic amplification primers

| Locus | Direction | Sequence (5′-3′) | SEQ ID NO: |
|---|---|---|---|
| GFP | For (oGH072) | AGGCCAGCTTGGCACTTGATGT | 82 |
| | Rev (oGH046) | TGTTGTGGCGGATCTTGAAGTTC | 83 |
| mCherry | For (oGH072) | AGGCCAGCTTGGCACTTGATGT | 84 |
| | Rev (oGH343) | GCTTCAGCCTCTGCTTGATCTC | 85 |
| Safe.2 | For (oGH371) | CACTATGACCACAGCCACTCAC | 86 |
| | Rev (oGH372) | CTTTCTGAAAAGTAACCCAGCCTCA | 87 |
| Safe.4 | For (oGH397) | GAACTGTGAATAATAAGCAATCATCCAG | 88 |
| | Rev (oGH398) | GCTTGCCAAAAATTGTGTACCCTTTCC | 89 |
| Safe.5 | For (oGH399) | TAGGTAACCCATCTGAGGTTTTCAAATAT | 90 |
| | Rev (oGH400) | GAGAAAAGAACATGACTTCCAGCAGC | 91 |
| Safe.6 | For (oGH401) | CCAAATTGCAGCCACACTTGAAAACC | 92 |
| | Rev (oGH402) | TAGGAAGCAGTGTAGGAGGATTGG | 93 |
| wtGFP | For (oGH072) | AGGCCAGCTTGGCACTTGATGT | 94 |
| | Rev (oGH029) | AAGCAGCGTATCCACATAGCGT | 95 |
| PSMB5 | For (oGH468) | GCAAGGGGGCTGGCTCCACAC | 96 |
| Exon 1 | Rev (oGH469) | TTAGTTCTTTCTGCCCACACTAGAC | 97 |
| PSBM5 | For (oGH470) | CATGTGGTTGCAGCTTAACTCAC | 98 |
| Exon 2 | Rev (oGH471) | GTGTTTTTGTGGTCTTATGTGGCC | 99 |
| PSMB5 | For (oGH472) | ACAACATACCACCCCATCTCACC | 100 |
| Exon 3 | Rev (oGH473) | CAAAGTGCTGGGATTACGGGTTTG | 101 |
| PSMB5 | For (oGH474) | CAAGCAGCTGCATCCACCCTCTT | 102 |
| Exon 4 | Rev (oGH475) | CTGCTAACCTCATCTCCCTTTCCAG | 103 |
| HBG2 | For (oGH440) | GTATCTTCAAACAGCTCACACCC | 104 |
| | Rev (oGH441) | GTCTTAGAGTATCCAGTGAGGCC | 105 |
| GSTP1 | For (oGH442) | CACTGAGGTTACGTAGTTTGCCC | 106 |
| | Rev (oGH443) | CGACAAATCCTCCTCCACCTCT | 107 |
| FTL | For (oGH454) | TTCCTCTCCGCTTGCAACCTCC | 108 |
| | Rev (oGH455) | CGGCACATAGAACTAAACCTACATTTC | 109 |
| PTPRC | For (oGH500) | GCCAGTAAGCATTTTCCTAATAGATGGAC | 110 |
| Locus 1 | Rev (oGH501) | GCCAAATGCCAAGAGTTTAAGCC | 111 |
| PTPRC | For (oGH502) | TCATCCTTCTGAACTCAATTGCTTTG | 112 |
| Locus 2 | Rev (oGH503) | CAATGATGCAAATGCTCTTAAAAGAAACTC | 113 |
| CD274 | For (oGH504) | GGTGACTATTTCATTTGTGTGACACTC | 114 |
| Locus 1 | Rev (oGH505) | GAAAGCAGTGTTCAGGGTCTACC | 115 |
| CD274 | For (oGH508) | GAAAACCTGAACAAATGGAGAGGG | 116 |
| Locus 2 | Rev (oGH509) | GCTTGCTCAGTAGATTATAATCCTACAGG | 117 |
| CD14 | For (oGH510) | GGTCGATAAGTCTTCCGAACCTC | 118 |
| | Rev (oGH511) | GCGAAACTGGTGAGTTACTAATTAATCC | 119 |

TABLE 5
PSMB5 variant installation
sgRNAs

| Mutation | sgRNA sequence (5′-3′) | SEQ ID NO: |
|---|---|---|
| L11L, Exon 1 Control | CCGCGCTGGTTCACCGGTAG | 120 |
| Intronic | CTGCAACTATGACTCCATGG | 121 |
| R78N, A79TG | TCATAGTTGCAGCTGACTCC | 122 |
| (Exon 2 Control) | | |
| G82D | AGCTGACTCCAGGGCTACAG | 123 |
| A108V | CTGCTAGGCACCATGGCTGG | 124 |
| G242D | CAACCTCTACCACGTGCGGG | 125 |
| Exon 4 Control | TGAAGGGAACCGGATTTCAG | 126 |

ssDNA donor oligonucleotides

| Mutation | Donor oligonucleotide sequence (5′-3′) | SEQ ID NO: |
|---|---|---|
| L11L (oGH512) | CAGATCTGCACGACCCCCAAGTCCGAAAAACCCGCGCTGGTT | 127 |
| | CACCGGTAACGGTCTCTCCAACACGCTGGCAAGCGCCATGTC | |
| | TAGTGTGGGCAGAAAG | |
| Exon 1 Control (oGH513) | CTCCCTGGACCTAGATCCAGCAGATCTGCAcGAccccCAAGT | 128 |
| | CCGAAAAATCCGCGCTGGTTCACCGGTAGCGGTCTCTCCAAC | |
| | ACGCTGGCAAGCGCCAT | |
| Intronic (oGH520) | ACCCGCTGTAGCCCTGGAGTCAGCTGCAAcTATGAcTcCATG | 129 |
| | GCGGAACTATTAAGATCAGAGGAAAACACAAAACAGGCCACA | |
| | TAAGACCACAAAAACAC | |
| R78N (oGH518) | CTATCACCTTCTTCACCGTCTGGGAGGCAATGTAAGcACCCG | 130 |
| | CTGTAGCCTTGGAGTCAGCTGCAACTATGACTCCATGGCGGA | |
| | ACTGTTAAGATCAGAGG | |
| A79T (oGH517) | CTCTATCACCTTCTTCACCGTCTGGGAGGCAATGTAAGCACC | 131 |
| | CGCTGTAGTCCTGGAGTCAGCTGCAACTATGACTCCATGGCG | |
| | GAACTGTTAAGATCAGA | |
| A79G (oGH516) | TCTCTATCACCTTCTTCACCGTCTGGGAGGcAATGTAAGCAC | 132 |
| | CCGCTGTACCCCTGGAGTCAGCTGCAACTATGACTCCATGGC | |
| | GGAACTGTTAAGATCAG | |
| G82D (oGH515) | ATGGGTTGATCTCTATCACCTTCTTCACcGTcTGGGAGGCAA | 133 |
| | TGTAAGCATCCGCTGTAGCCCTGGAGTCAGCTGCAACTATGA | |
| | CTCCATGGCGGAACTGT | |
| A108V (oGH514) | AGATTCGACATTGCCGAGCCAACAGCCGTTcccAGAAGCTGC | 134 |
| | AATCCGCTACGCCCCCAGCCATGGTGCCTAGCAGGTATGGGT | |
| | TGATCTCTATCACCTTC | |
| Exon 2 Control (oGH519) | ATCTCTATCACCTTCTTCACCGTCTGGGAGGcAATGTAAGCA | 135 |
| | CCCGCTGTCGCCCTGGAGTCAGCTGCAACTATGACTCCATGG | |
| | CGGAACTGTTAAGATCA | |
| G242D (oGH521) | TATACTTCTCATGTAGATCAGCCACATTGTcAcTGGAGACTC | 136 |
| | GGATCCAGTCATCCTCCCGCACGTGGTAGAGGTTGACTGCAC | |
| | CTCCTGAGTAGGCATCT | |
| Exon 4 Control (oGH523) | TCCATGACCCCATATGCATACACAGAGCCAGAAccTACAGAG | 137 |
| | AAGGTGGCACCTGAAATCCGGTTCCCTTCACTGTCCACGTAG | |
| | TAGAGGCCTGGAAAGGG | |

Lenti dCAS-VP64_Blast, lenti MS2-P65-HSF1_Hygro, and lenti sgRNA(MS2)_zeo backbone were a gift from Feng Zhang (Addgene plasmids #61425-61427). The VP64 effector was removed from the dCas9 construct by digesting with BamHI and EcoRI followed by Gibson assembly to re-insert PCR amplified blasticidin resistance marker (pGH125). For MS2 fusions, P65-HSF1 was removed using restriction digest with BamHI and BsrGI. AID (pGH156) and AIDΔ (pGH153) were PCR amplified from a FLAG-AID expressing plasmid, courtesy of the Cimprich Lab, and Gibson assembled into the digested vector. Catalytically inactive (pGH183) and hyperactive mutants (pGH335) were generated using PCR primers containing the desired mutations. Subunits of AID were amplified using those primers and then joined using overlapping PCR. The mutant AID PCR product was Gibson assembled into the digested MS2 expression vector. GFP, mCherry, and wtGFP expressing plasmids driven by an Ef1α promoter were generated using pMCB246 digested with Nhe1 and Xba1, removing a puromycin resistance-T2A-mCherry cassette. GFP (pGH045) and mCherry (pGH044) were PCR amplified and inserted into the digested vector using Gibson assembly. Variants of GFP (wtGFP (pGH220)) and identified mutants (pGH311-565T, pGH312-Q80H, pGH314-S65T+Q80H) were constructed using the previously described overlapping PCR method followed by Gibson assembly. For dual guide experiments, a second sgRNA expressing plasmid was constructed by removing the zeocin resistance (digestion of lenti sgRNA(MS2)_zeo with BsrGI and EcoRI) and replaced with puromycin resistance with a removed BsmBI cut site by Gibson assembly (pGH224). sgRNA vectors were generated by digesting either lenti sgRNA(MS2)_zeo or pGH224 with BsmBI. Oligonucleotides with overhangs compatible with subsequent ligation were designed and annealed followed by ligation into the digested vector. The sequences for the sgRNAs are listed in the Tables, e.g., Tables 3, 5, and 6A. All plasmid sequences were verified using Sanger sequencing. All oligonucleotides were ordered from Integrated DNA Technologies (IDT).

### Cell Culture and Generating Parent Cell Lines

Lentiviral production as well as infection and culturing of K562 cells (ATCC) were performed as described (45). Parental K562 cell lines were generated by infecting dCas9-Blast (pGH125) followed by blasticidin selection (10 μg/mL, Gibco) for 7 days. Cells were subsequently infected with both GFP (pGH045) and mCherry (pGH044) expression vectors or with a wtGFP (pGH220) expression vector and sorted via FACS for fluorescence. These cell lines were used as the parental samples in the sequencing assays. For experiments using an integrated construct, cells were infected with MS2-AID (pGH153, 156, 183, and 335) expressing vectors followed by selection with hygromycin B (200 μg/mL, Life Technologies) for 7 days. All cell lines were maintained in a humidified incubator (37° C., 5% CO₂), and checked regularly for mycoplasma contamination.

### Fluorescence Microscopy of MS2-A1D Localization

K562 cells were lentivirally infected by constructs expressing an MS2-AID (pGH153 and pGH156) and selected with hygromycin B for 7 days. 1 million cells were harvested and fixed in 4% paraformaldehyde for 15 min at room temperature. Cells were washed 3 times with PBS and then permeabilized with 0.1% Triton-X in PBS for 10 minutes at 4° C. Cells were incubated in blocking solution (3% BSA in PBS) for 1 hour at room temperature. They were centrifuged at 500×g for 5 minutes and resuspended in 1:500 dilution of rabbit anti-MS2 antibody (Millipore, cat no. ABE76) in blocking solution for 2 hours at room temperature. The cells were washed 3 times with PBS and resuspended in 1:1000 dilution of Alexa Fluor 488 conjugated goat anti-rabbit antibody (Life Technologies) in blocking solution and incubated for 2 hours at room temperature. Cells were washed in PBS 3 times and resuspended in Vectashield (Vector Laboratories) containing DAPI. The samples were deposited on a glass coverslip and imaged using an inverted Nikon Eclipse Ti confocal microscope with 488 nm (AlexaFluor488) and 405 nm (DAPI) lasers, an oil immersion objective (Plan Apo λ, N.A.=1.5, 100×, Nikon), and an Andor Ixon3 EMCCD camera. Images were processed using ImageJ (National Institutes of Health).

### Transfection of K562 Cells and Testing MS2-AID Variants

Nucleofection of K562 cells was performed as described (46). 1 million K562 cells were harvested for each electroporation. Cells were centrifuged at 300×g for 5 minutes and resuspended in 100 μL of nucleofection solution and mixed with plasmid DNA (5 μg MS2-AID expressing plasmid and 5 μg sgRNA expression vector) and loaded into a 2 mm cuvette (VWR). Electroporations were performed using the T-016 program on the Lonza Nucleofector 2b. After electroporation, cells were rescued in warm, supplemented RPMI media. Cells were grown for 10 days and the GFP and mCherry fluorescence were measured using the BD Accuri C6 flow cytometer. Scatter plots were generated in FlowJo. The cells were sorted for low GFP fluorescence and the cells were grown before preparation of sequencing.

Generating Mutations from Individual and Dual sgRNA Experiments

For experiments using integrated constructs, three days after infection, selection was applied and continued for 11 days using blasticidin for dCas9, hygromycin B for MS2-AID variants, and zeocin (200 μg/mL, Life Technologies) for sgRNA. For dual sgRNA experiments, the sgGFP.10 plasmid was further selected using puromycin (1 μg/mL, Sigma-Aldrich). For GFP and mCherry targeting sgRNAs, the GFP and mCherry fluorescence were measured after selection using a BD Accuri C6 flow cytometer. Scatter plots were generated in FlowJo. Experiments targeting GFP or mCherry were performed with 3 biological replicates while endogenous loci were performed with 2 biological replicates.

### Preparation of Sequencing Samples

To sequence targeted loci, genomic DNA was extracted from 0.5-1.5 million cells using the QiaAmp DNA mini kit (Qiagen). The targeted loci were PCR amplified from 0.5-1.0 μg of genomic DNA using primers shown in Table 4. The product was purified on a 0.8-1% TAE agarose gel. The concentration was measured by Qubit (Life Technologies) and then prepared for sequencing following the Nextera XT kit protocol (Illumina). For PSMB5 experiments, DNA was extracted from 20 million cells and PCR amplification was performed on 5 μg of genomic DNA. After individual gel purification of PCR product from each exon, PCR products were mixed in equimolar amounts before beginning the Nextera XT preparation. Sequences were measured on a NextSeq 500 (Illumina) with paired end reads of length 76 or 151 bp. Every sequencing run included a parental sample for each locus that was being sequenced.

### Analysis of Sequencing Data—Sample Sequencing and Alignment

A number of 4.5 million reads was produced on average over all sequenced samples. Sequencing adapters (5′ adapter: CTGTCTCTTATACACATCTCCGAGCCCACGAGAC (SEQ ID NO: 2); 3′ adapter: CTGTCTCTTATACACATCTGACGCTGCCGACGA (SEQ ID NO: 3)) were trimmed using cutadapt (version 1.8.1 (47)), also discarding reads under 30 bp and nucleotides flanking the adapters with Illumina quality score lower than 30 (leaving only flanking sequences for which the base call accuracy is over 99.9%). Alignment on respective reference loci was performed using bwa aln (v0.7.7) and bwa samse (48). A maximum number of 3 or 5 mismatches was allowed for samples with read length of 76 bp and 151 bp respectively. Aligned files were then sorted using samtools (v0.1.19 (49))

Reads aligned to their respective references with mapping quality over 30 were kept for further analysis. On average, 90% of sequenced reads (Standard Deviation 16%) were successfully mapped to the provided reference genome. From these aligned reads, 96% (Standard Deviation 5.7%) were remaining after filtering on mapping quality.

### Analysis of Sequencing Data—Tabulation of Mutations Per Base

Allelic counts at each position were calculated with a custom script applied to data after filtering for nucleotides with Illumina base quality score over 30 using samtools mpileup (version 1.2). The parental sample was used to estimate the mutations introduced through sample preparation and sequencing. Using the parental as a reference, the mutation enrichment was calculated at each base by taking the percentage of reads with alternative alleles in comparison to the same proportion calculated in the parental sample. The first and last 50 bases of each locus were excluded from these enrichments because the ends had lower read coverage that was a byproduct of the Nextera XT preparation. Transitions, transversions, and indels observed in hotspots were determined by evaluating the distribution of frequencies of every possible alternative nucleotide at each position. Parental cell line respective frequencies in the hotspots were then subtracted to account for background noise. Negative values were set to 0. The standard deviation of the frequency of alternative alleles in all parental samples from the studied batch was used to estimate the remaining noise resulting from sequencing and variability between samples. Reported medians, maximums, and distributions result from this calculation.

### Calculation of Mutation Frequency in Hotspot Regions

The number of mutations per read was limited during the alignment step (see above). Mutation counts were performed using the filtered aligned data to compute the enrichment of reads carrying mutations within the hotspot. After selecting all reads overlapping the hotspot using samtools view (version 1.2 (49)), each read was screened for mutations with their respective positions. These results were then summarized for each sample by calculating the ratio between the number of reads with mutations spanning the hotspot and the total number of reads spanning the hotspot. The frequency of mutations enrichment was calculated by subtracting the results from the parental cell line as background.

Evolution of wtGFP to EGFP

For transfected wtGFP experiments, K562 cells expressing dCas9 and wtGFP were nucleofected as described earlier with 5 μg of MS2-AIDΔ and either 1.25 μg for each of wtGFP.1-4 or Safe.2,4-6 sgRNA expressing vectors. Cells were grown for 10 days after electroporation before sorting. For integrated experiments, K562 cells expressing dCas9, MS2-AIDΔ, and wtGFP were infected with either wtGFP.1 or Safe.2 sgRNA expressing vectors. After 3 days, cells were selected with blasticidin, hygromycin B, and zeocin for 11 days. Cells were sorted via FACS to obtain spectrum-shifted GFP variants. For the electroporation experiments, cells were grown for 7 days between sorting rounds. Samples were prepared for sequencing as described previously.

Flow Cytometry of wtGFP Variants

HEK293T (ATCC) cells were cultured in DMEM with 10% FBS, penicillin/streptomycin, and L-glutamine. For each transfection, 1 million HEK293T cells were plated in 2 mL of supplemented DMEM media. 1.5 μg of wtGFP expressing plasmid (pGH045, 220, 311, 312, and 314) was mixed with 200 μL serum-free DMEM and 10 μL of polyethylenimine (PEI, 1 mg/mL, pH 7.0, PolySciences Inc.) and incubated at room temperature for 30 minutes. The mixture was added to the cells and grown for 72 hours with an additional 3 mL of DMEM supplemented media added after 24 hours. The samples were trypsinized and analyzed using a FACScan flow cytometer (BD Biosciences). Additional analysis of the data was performed using FlowJo.

### Design and Construction of PSMB5 Tiling Libraries

The PSMB5 tiling library was generated using CHOPCHOP online tool (50) for the three PSMB5 isoforms (NCBI accession NM_0011449632, NM_00130725, and NM_002797). sgRNAs for each isoform were combined. sgRNAs having any genomic off-target matches, more than 1 off-target when allowing one mismatch in the sgRNA sequence, or 5 or more off-targets when allowing one or two mismatches within the sgRNA sequence were removed. The sgRNAs were further filtered by removing any containing a BsmBI cut site, which interferes with the library cloning strategy. The final library contained 143 sgRNAs (Table 6A). Safe harbor sgRNAs were designed to target genomic loci that have not been annotated to include gene exons or UTRs, have signal in biochemical assays (DNaseI, CHIP-Seq, etc.), or have signal in sequence-based analyses (conserved elements, transcription factor motif searches, etc.). 705 sgRNAs targeting safe harbor regions were selected to serve as a control library. The sgRNA sequences for both libraries are included in Tables 6A and 6B.

Oligonucleotide libraries were synthesized by Agilent and cloned into the sgRNA expression vector as previously described (51-53). Vector and sgRNA inserts were digested with BsmBI. Large scale lentivirus production and infection of K562 cells were performed as described (51, 52). Three days after infection, selection began with blasticidin, hygromycin B, and zeocin for 11 days. Cells were expanded to 20 million cells for each treatment (safe harbor and PSMB5 libraries in duplicate) and were pulsed with 20 nM bortezomib (Fisher Scientific) for three days followed by recovery until log growth was restored (5-10 days) before the next pulse. The cells were pulsed a total of three times. After the final pulse, cells were harvested and prepared for sequencing as described earlier.

### Installation and Validation of Bortezomib Resistant PSMB5 Mutations

sgRNAs were designed to target near the location of the installed SNP and 101-nt donor oligos were designed to be centered around the installed mutation. Oligonucleotides with proper overhangs were ordered from IDT and annealed before ligation into BbsI digested pGH020, a hu6 driven sgRNA expression vector. All plasmids were verified by Sanger sequencing. The sgRNA and ssDNA donor oligo sequences are listed in Table 5.

K562 cells expressing Cas9 were electroporated with 5 μg of sgRNA expressing vector and 100 picomoles of donor oligo. Cells were grown for 6 days before 300,000 cells were placed under selection with 20 nM bortezomib for 14 days. The viability of the cells was measured by flow cytometry using a live cell gate (FSC/SSC). After selection, 750,000 cells were harvested and genomic DNA was extracted using the QiaAmp DNA Mini Kit (Qiagen). The PSMB5 exonic locus containing the mutation was PCR amplified, gel purified, and ligated into the pCR-Blunt vector using the Zero-Blunt cloning kit (Life Technologies). 8-15 colonies were Sanger sequenced for each sample.

### Example 2—Targeted Mutagenesis Through dCas9 Recruitment of AID

To recruit the AID protein to a genetic locus, a dCas9 (28) protein and a single guide RNA (sgRNA) comprising one or more MS2 hairpin binding sites was used (FIG. 1) (18). In this system, the sgRNA contains two MS2 hairpins that each recruit two MS2 proteins (four in total) fused to AID. However, the technology is not limited to this particular arrangement and embodiments comprise an sgRNA comprising 1 or more (e.g., 1, 2, 3, 4, 5, 6 or more) hairpins for recruiting MS2 protein fusions to a genetic locus.

For the initial test, MS2 was fused to three AID variants (FIG. 2): 1) wild-type AID; 2) a truncated version without the last three amino acids (AIDΔ), which is a mutant protein lacking a functional nuclear export signal (NES) and having increasing SHM activity (30); and 3) a catalytically inactive truncated version (AIDΔDead) (31). Fluorescence microscopy was used to visualize the MS2-AID and MS2-AIDΔ constructs in K562 cells. Cells were fixed and stained with an MS2 antibody and the nuclear stain DAPI. Images indicated that the deletion of the NES resulted in primarily nuclear localization of the MS2 fusion protein as observed by immunofluorescence staining in K562 cells.

K562 cells were generated that stably expressed dCas9 along with GFP and mCherry, which, when used together with sgRNAs targeting GFP, served as a phenotypic readout for on-target (GFP) and off-target mutations (mCherry). These cells were transfected with plasmids coding for either a GFP-targeting sgRNA (sgGFP.1) or a scrambled non-targeting sgRNA (sgNegCtrl) paired with plasmids coding for MS2-AID, MS2-AIDΔ, or MS2-AIDΔDead. After 10 days, cells were analyzed by flow cytometry to measure GFP and mCherry fluorescence. GFP and mCherry fluorescence of the cells were measured by flow cytometry as a proxy for mutation rate. As expected for on-target mutations resulting in non-fluorescent protein, an increase in the GFP negative population was observed for MS2-AIDΔ treatment when comparing sgGFP.1 to sgNegCtrl (1.64% vs. 0.55%). However, this effect was not observed with MS2-AID (0.71% vs. 0.78%). At the same time, the mCherry negative population showed little change (1.02% vs. 0.91%), indicating that targeting AIDΔ to GFP resulted in specific mutagenesis.

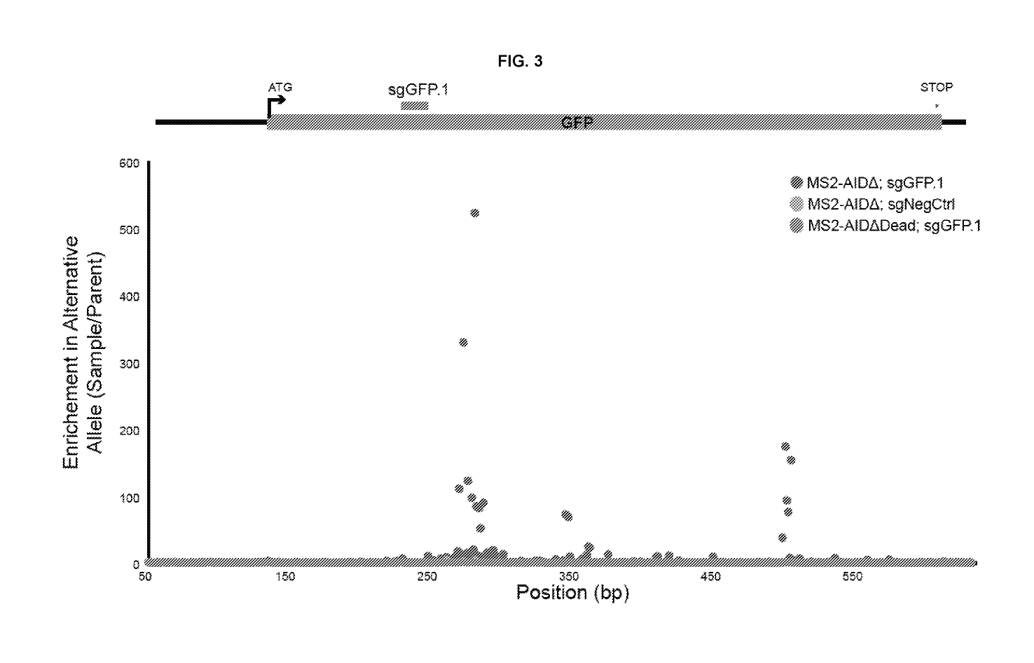

Based on the observed change in fluorescence, a more detailed analysis of the population was performed by sequencing the locus. To quantify mutations in the GFP negative population, the GFP low population was collected from the AIDΔ:sgGFP.1, AIDΔ:sgNegCtrl, and AIDΔ-Dead:sgGFP.1 samples via FACS and the GFP locus was sequenced. Enrichment of mutations was calculated by comparing collected samples to parental cells that had not been exposed to a mutagenic agent. Enrichment of mutations was observed only in the AIDΔ:sgGFP.1 (FIG. 3). The most enriched position for mutations was base pair 280 which had over 500-fold enrichment in mutations and 41.2% of sequences at that base showed a G>A transition (FIG. 3). This transition resulted in the introduction of a tyrosine in place of cysteine in GFP at amino acid 48. Reduced fluorescence of GFP due to this alteration is consistent with previous work showing that cysteine thiol binding by dTNB quenches GFP fluorescence (32).

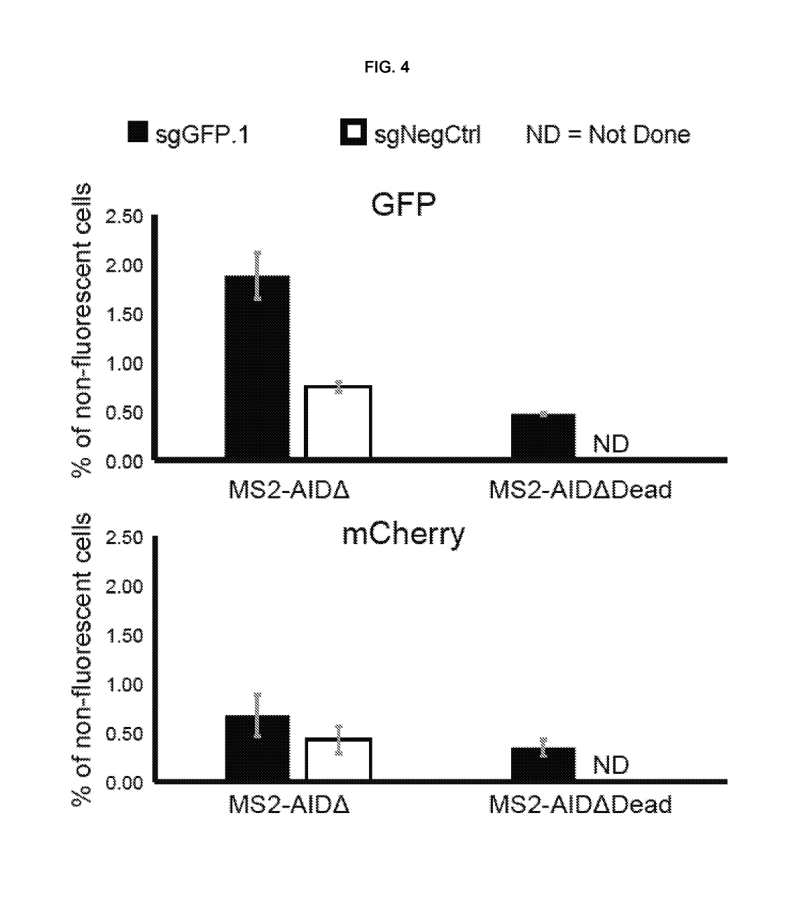

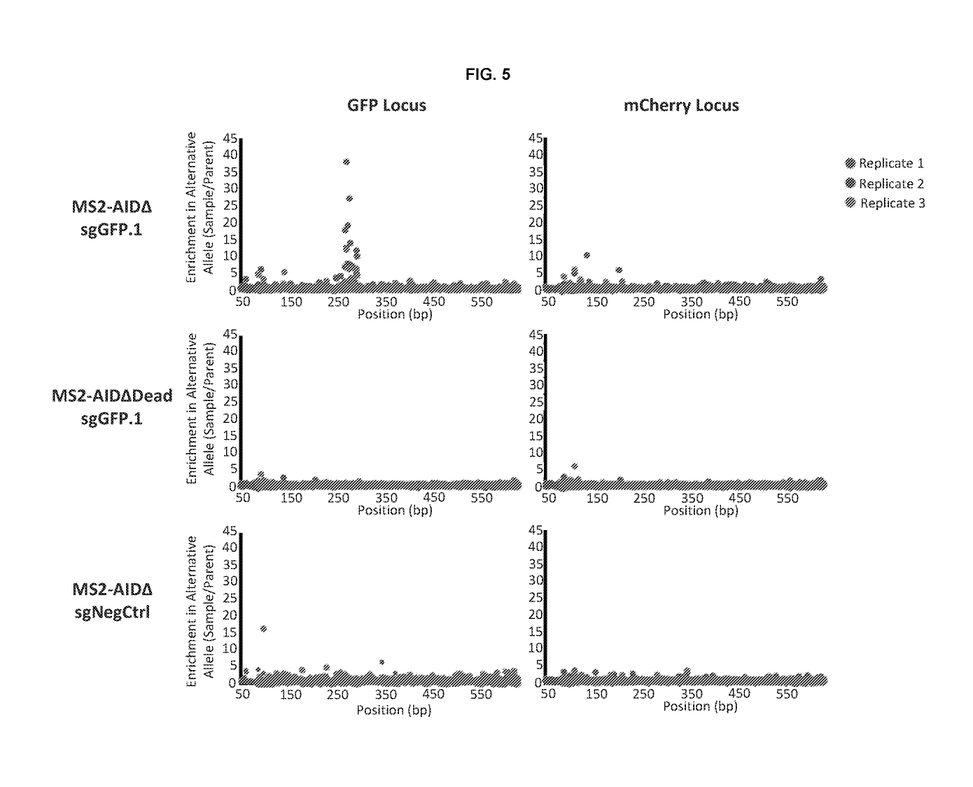

Given the superior performance of AIDΔ, experiments were continued with this AID variant. The mutation rate was estimated by integrating the constructs into reporter cells, which minimized experimental variation due to transfection efficiency. MS2-AIDΔ or MS2-AIDΔDead was stably integrated in cells together with sgGFP.1 or sgNegCtrl, and GFP and mCherry negative populations were monitored 14 days after infection. GFP and mCherry fluorescence of the cells was measured by flow cytometry as a proxy for mutation rate. As before, in the presence of MS2-AIDΔ, an increase in the GFP negative population was observed (1.88%) when compared to either the sgNegCtrl (0.75%) or MS2-AIDΔDead (0.47%). By contrast, the mCherry low population was minimally changed (0.67% MS2-AIDΔ:sgGFP.1, 0.34% MS2-AIDΔ:sgNegCtrl, 0.43% MS2-AIDΔDead:sgGFP.1) (FIG. 4). Both GFP and mCherry loci from these cells were sequenced (FIG. 5), and an enrichment of mutations was observed in the 270-290 bp region of GFP only in cells expressing MS2-AIDΔ:sgGFP.1. Enrichment of mutations in the mCherry locus was not detected.

### Example 3—Defining the Region of Mutagenesis

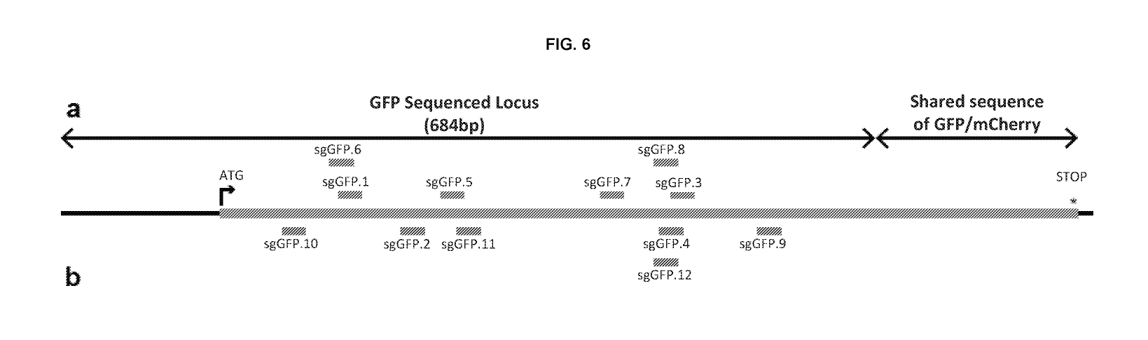

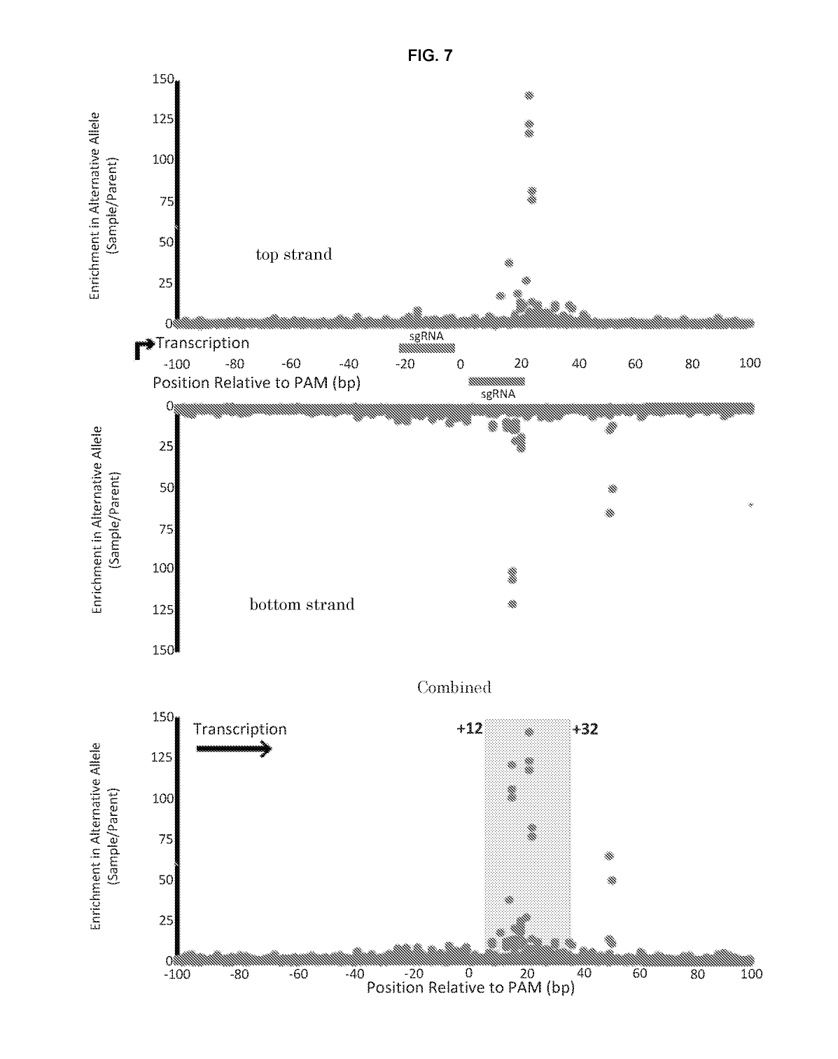

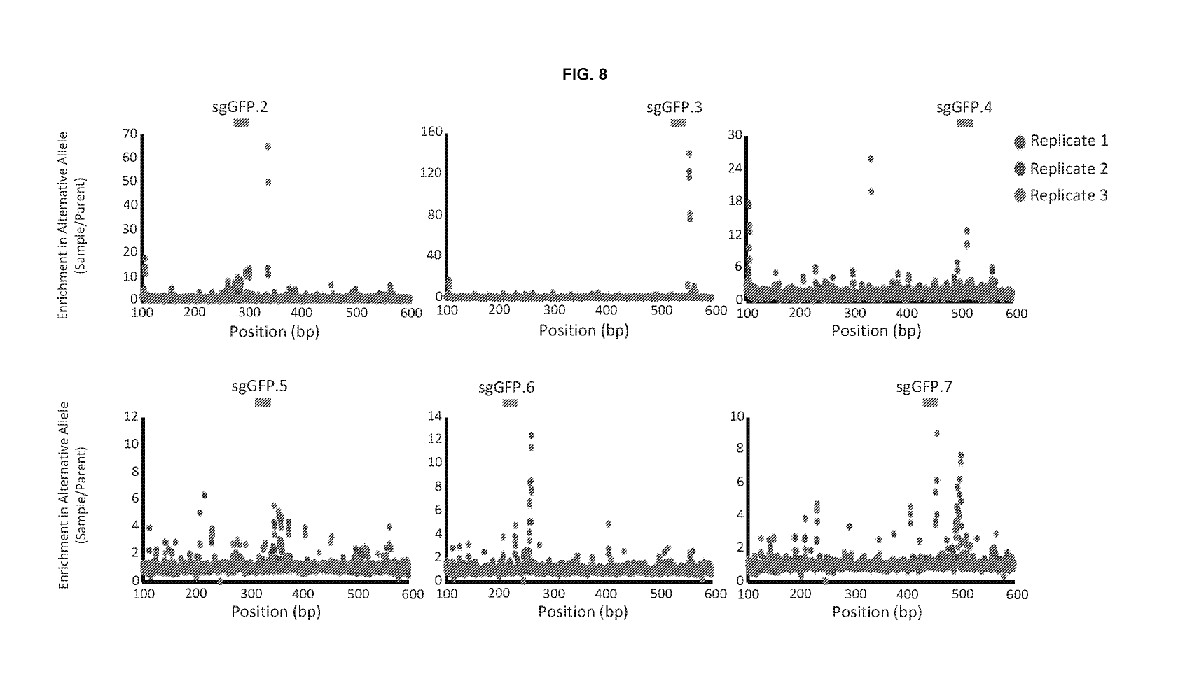

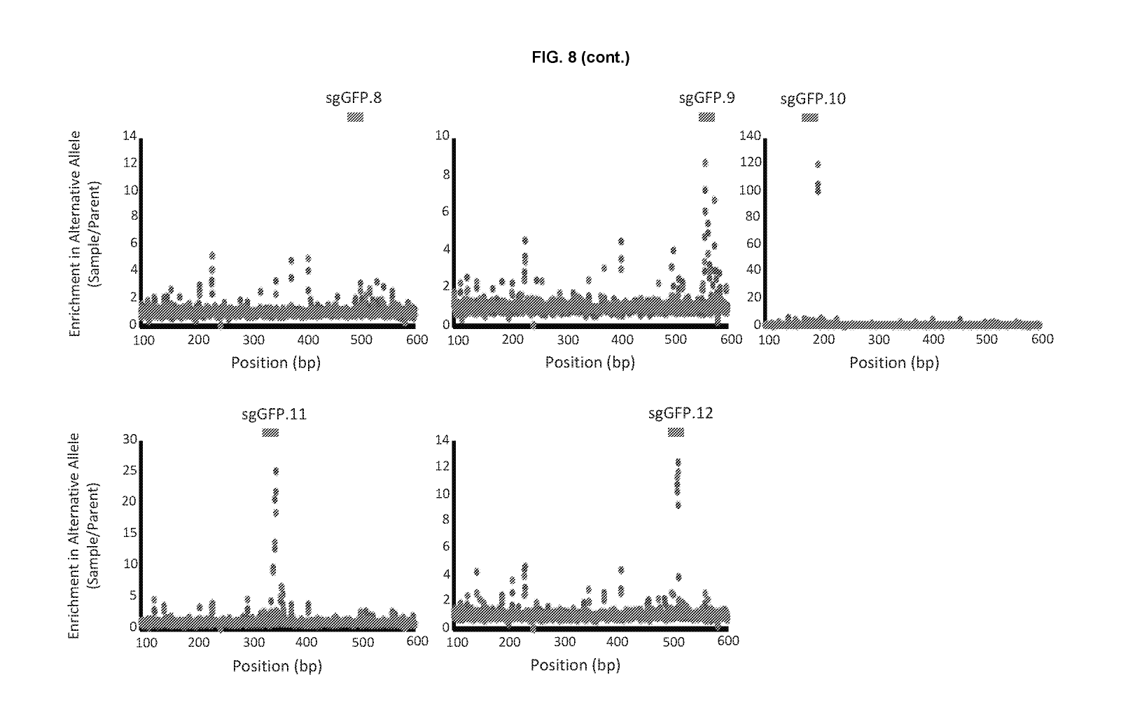

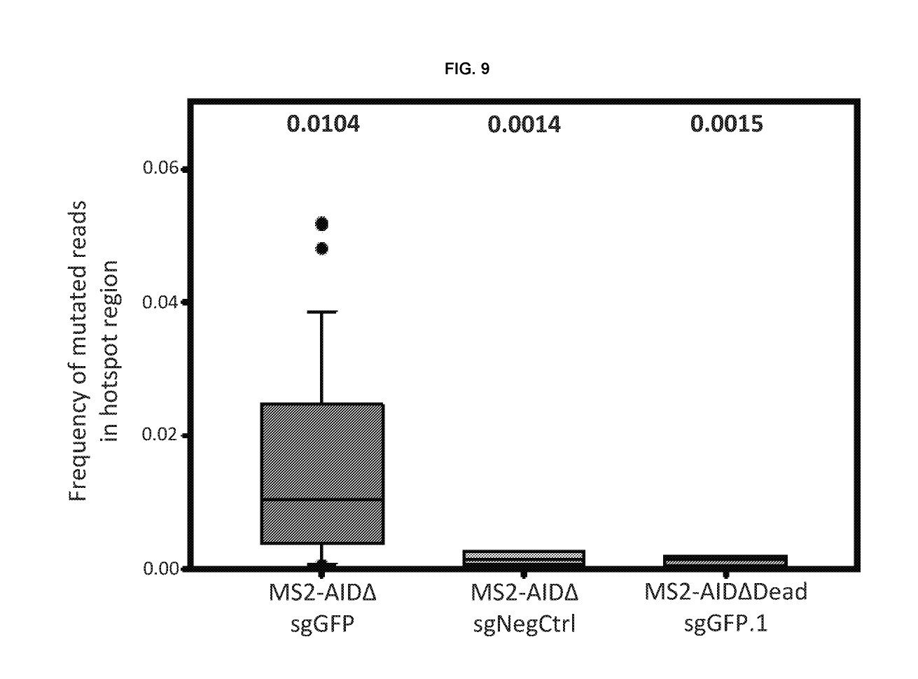

To determine the region of mutagenesis with respect to the sgRNA, an additional 11 sgRNAs (sgGFP.2-12) were selected that tiled the GFP locus on both strands (FIG. 6). Since AID mutagenesis has been shown to require transcription (12), it was contemplated that the strand of the guide relative to the direction of transcription may change the targeting of mutations. The GFP locus was sequenced in each of these samples and mutations were mapped relative to the end of the PAM sequence of each sgRNA (FIG. 7). While different sgRNAs exhibited a range of mutation efficiencies (FIG. 8), a mutational hotspot region was observed from +12 to +32 bp downstream of the PAM relative to the direction of transcription that was independent of the strand targeting (FIG. 7). The mutational hotspot was defined to include any base with at least 10-fold increased mutation over all three biological replicates for a given sgRNA. Mutations in this region were measured for the 12 sgGFP guides, and a mutation frequency of 0.0104 was observed (FIG. 9). This translates to a mutation rate of ˜1/2000 bp, which is similar to that observed for somatic hypermutation, and is an order of magnitude higher than the observed frequency of 0.0014 for a negative control sgRNA (M52-AIDΔ:sgNegCtrl) and 0.0015 for catalytically inactive AID (MS2-AIDΔDead:sgGFP.1). Given the ability of this system to generate targeted point mutations, additional experiments were conducted in which the technology was tested for directed evolution.

### Example 4—Evolution of wtGFP to EGFP

Experiments were conducted to alter an integrated copy of wild-type GFP (wtGFP) from Aequorea victoria (excitation 395 nm/emission 509 nm) to produce EGFP (excitation 490/emission 509 nm) (33). EGFP has two substituted residues relative to wtGFP: S65T, which shifts the excitation/emission spectrum, and F64L, which improves the folding kinetics of GFP (33-35). Four guides were designed (sgwtGFP.1-4) that target this region and the guides and MS2-AIDΔ were transfected into K562 cells expressing dCas9 and wtGFP. As a negative control, four “safe harbor” sgRNAs were also transfected that target regions of the genome that are annotated as non-functional. Cells were grown for 10 days to allow for mutations to be introduced, and then cells were sorted by FACS to collect cells expressing spectrum-shifted GFP. In biological replicate experiments, a population was observed with decreased signal in the Pacific Blue channel and increased GFP signal (0.076% replicate 1, 0.025% replicate 2), which was not observed in the safe harbor samples (0.002%, 0.002%). After another round of sorting, the safe harbor samples did not have any cells pass the sorting gates, while the spectrum-shifted population had increased to 2.29% and 1.16% in the GFP-targeted replicates.

The GFP locus was sequenced to identify mutations enriched by the sorting process, revealing enrichment of mutations at positions 331 (G>C) and 377 (G>C). The former mutation introduces the known S65T mutation from EGFP. The latter mutation generated a Q80H substitution, which was suspected to be a passenger mutation since the majority of sequences containing the mutation also showed the S65T transition. Each mutation was introduced into GFP separately, and it was confirmed that the S65T mutation alters the fluorescence spectrum of GFP while Q80H does not, either alone or in conjunction with S65T. A similar selection experiment that was performed with the integrated constructs and a single integrated guide (sgwtGFP.1 or sgSafe.2) recovered the same S65T transition but did not observe the Q80H mutation.

### Example 5—Identification of Bortezomib-Resistant PSMB5 Variants

Another potential application of the technology is the investigation of mechanisms of drug resistance. Mutations are a common escape pathway for cancer cells to develop resistance to drug treatment (36), and understanding which mutations can arise is important for the design of new drugs or drug combinations. To test this, PSMB5 was mutagenized. PSMB5 is a core subunit of the 20S proteasome, which is the target of the proteasome inhibitor bortezomib (37). A library of 143 guides was generated tiling all coding exons of PSMB5 (Table 6A). A control library of 705 safe harbor guides was also generated (Table 6B).

TABLE 6A
PSMB5 tiling library

| | | SEQ |
|---|---|---|
| | | ID |
| sgRNA Name | sgRNA sequence | NO: |
| PSMB5_001144932.23 | AAAAACCCGCGCTGGTTCAC | 847 |
| PSMB5_001144932.36 | AACAACCACCCTGGCCTTCA | 848 |
| PSMB5_00130725.83 | AACATGGTGTATCAGTACAA | 849 |
| PSMB5_001144932.101 | AAGGTAGTTATTATAATATA | 850 |
| PSMB5_001144932.107 | AAGTACATTCCAAATGACTT | 851 |
| PSMB5_00130725.84 | AATCTATGAGCTTCGAAATA | 852 |
| PSMB5_00130725.60 | ACCACGTGCGGGAGGATGGC | 853 |
| PSMB5_00130725.47 | ACCTGCTAGGCACCATGGCT | 854 |
| PSMB5_00130725.29 | ACGTAGTAGAGGCCTGGAAA | 855 |
| PSMB5_00130725.52 | ACGTGGACAGTGAAGGGAAC | 856 |
| PSMB5_00130725.36 | AGAAGGTGGCCCCTGAAATC | 857 |
| PSMB5_001144932.29 | AGACCATCACTGAGACTCCC | 858 |
| PSMB5_00130725.78 | AGAGCCAGAACCTACAGAGA | 859 |
| PSMB5_001144932.59 | AGAGGATCGGCAACATGGCA | 860 |
| PSMB5_001144932.97 | AGCCTGGCCGCGCCAGGCTG | 861 |
| PSMB5_001144932.27 | AGCGCGGGTTTTTCGGACTT | 862 |
| PSMB5_001144932.9 | AGCTGACTCCAGGGCTACAG | 863 |
| PSMB5_00130725.61 | AGCTGCATCCACCCTCTTTC | 864 |
| PSMB5_00130725.67 | AGGCATCTCTGTAGGTGGCT | 865 |
| PSMB5_00130725.44 | AGTCAACCTCTACCACGTGC | 866 |
| PSMB5_00130725.34 | AGTGAAGGGAACCGGATTTC | 867 |
| PSMB5_00130725.80 | AGTGGAGCAGGCCTATGATC | 868 |
| PSMB5_00130725.19 | ATCCGCTGCGCCCCCAGCCA | 869 |
| PSMB5_001144932.90 | ATCTGCTGGATCTAGGTCCA | 870 |
| PSMB5_00130725.70 | ATCTGTGGCTGGGATAAGAG | 871 |
| PSMB5_00130725.39 | ATGCATATGGGGTCATGGAT | 872 |
| PSMB5_001144932.33 | ATTTCGATTCCTGGCTCTTC | 873 |
| PSMB5_00130725.24 | CAAAGGCATGGGGCTGTCCA | 874 |
| PSMB5_00130725.9 | CAACCTCTACCACGTGCGGG | 875 |
| PSMB5_001144932.25 | CAAGTCCGAAAAACCCGCGC | 876 |
| PSMB5_00130725.2 | CACCATGGCTGGGGGCGCAG | 877 |
| PSMB5_00130725.50 | CACCATGTTGGCAAGCAGTT | 878 |
| PSMB5_001144932.99 | CACCCCAGCCTGGCGCGGCC | 879 |
| PSMB5_001144932.10 | CACCTTCTTCACCGTCTGGG | 880 |
| PSMB5_00130725.30 | CACGTAGTAGAGGCCTGGAA | 881 |
| PSMB5_001144932.26 | CAGCGCGGGTTTTTCGGACT | 882 |
| PSMB5_001144932.39 | CAGCTGCAACTATGACTCCA | 883 |
| PSMB5_00130725.23 | CAGCTTCTGGGAACGGCTGT | 884 |
| PSMB5_00130725.8 | CAGTCAACCTCTACCACGTG | 885 |
| PSMB5_00130725.79 | CATAGGCCTGCTCCACTTCC | 886 |
| PSMB5_001144932.70 | CATAGTTGCAGCTGACTCCA | 887 |
| PSMB5_00130725.16 | CATCCTCCCGCACGTGGTAG | 888 |
| PSMB5_001144932.19 | CATGGCGCTTGCCAGCGTGT | 889 |
| PSMB5_00130725.3 | CATGTTGGCAAGCAGTTTGG | 890 |
| PSMB5_001144932.6 | CCACACCTTGAAGGCCAGGG | 891 |
| PSMB5_00130725.76 | CCACATTGTCACTGGAGACT | 892 |
| PSMB5_001144932.34 | CCATGAAGCATTTCGATTCC | 893 |
| PSMB5_00130725.18 | CCATGGTGCCTAGCAGGTAT | 894 |
| PSMB5_00130725.48 | CCCCAGCCATGGTGCCTAGC | 895 |
| PSMB5_001144932.2 | CCGCGCTGGTTCACCGGTAG | 896 |
| PSMB5_00130725.21 | CGCAGCGGATTGCAGCTTCT | 897 |
| PSMB5_001144932.4 | CGCGGGTTTTTCGGACTTGG | 898 |
| PSMB5_001144932.22 | CGCTACCGGTGAACCAGCGC | 899 |
| PSMB5_00130725.22 | CGGATTGCAGCTTCTGGGAA | 900 |
| PSMB5_001144932.28 | CGTGCAGATCTGCTGGATCT | 901 |
| PSMB5_001144932.21 | CGTGTTGGAGAGACCGCTAC | 902 |
| PSMB5_00130725.64 | CTAACCTCATCTCCCTTTCC | 903 |
| PSMB5_001144932.45 | CTATCACCTTCTTCACCGTC | 904 |
| PSMB5_00130725.56 | CTATGACCTGGAAGTGGAGC | 905 |
| PSMB5_00130725.14 | CTATTCCTATGACCTGGAAG | 906 |
| PSMB5_00130725.59 | CTCTACCACGTGCGGGAGGA | 907 |
| PSMB5_00130725.11 | CTCTACCCCCTGAAAGAGGG | 908 |
| PSMB5_00130725.32 | CTCTACTACGTGGACAGTGA | 909 |
| PSMB5_001144932.8 | CTGCAACTATGACTCCATGG | 910 |
| PSMB5_00130725.13 | CTGCATCCACCCTCTTTCAG | 911 |
| PSMB5_00130725.1 | CTGCTAGGCACCATGGCTGG | 912 |
| PSMB5_00130725.55 | CTGCTCCACTTCCAGGTCAT | 913 |
| PSMB5_00130725.65 | CTGGCTCTGTGTATGCATAT | 914 |
| PSMB5_00130725.31 | CTGTCCACGTAGTAGAGGCC | 915 |
| PSMB5_00130725.26 | CTTATCCCAGCCACAGATCA | 916 |
| PSMB5_00130725.5 | CTTCACTGTCCACGTAGTAG | 917 |
| PSMB5_00130725.4 | CTTTCCAGGCCTCTACTACG | 918 |
| PSMB5_001144932.17 | CTTTCTGCCCACACTAGACA | 919 |
| PSMB5_001144932.72 | GAGATCAACCCATACCTGCT | 920 |
| PSMB5_001144932.102 | GAGCCTGGCCGCGCCAGGCT | 921 |
| PSMB5_00130725.85 | GATCTACATGAGAAGTATAG | 922 |
| PSMB5_001144932.94 | GATCTGCTGGATCTAGGTCC | 923 |
| PSMB5_001144932.18 | GCAAGCGCCATGTCTAGTGT | 924 |
| PSMB5_00130725.7 | GCATATGGGGTCATGGATCG | 925 |
| PSMB5_00130725.63 | GCCACAGATCATGGTGCCCA | 926 |
| PSMB5_00130725.37 | GCCACCTTCTCTGTAGGTTC | 927 |
| PSMB5_00130725.71 | GCCAGAACCTACAGAGAAGG | 928 |
| PSMB5_00130725.62 | GCCATGGTGCCTAGCAGGTA | 929 |
| PSMB5_00130725.20 | GCGCAGCGGATTGCAGCTTC | 930 |
| PSMB5_001144932.3 | GCGCGGGTTTTTCGGACTTG | 931 |
| PSMB5_001144932.69 | GCTCCACACCTTGAAGGCCA | 932 |
| PSMB5_001144932.71 | GCTGACTCCAGGGCTACAGC | 933 |
| PSMB5_00130725.46 | GCTGCATCCACCCTCTTTCA | 934 |
| PSMB5_001144932.35 | GCTTCATGGAACAACCACCC | 935 |
| PSMB5_001144932.1 | GGCAAGCGCCATGTCTAGTG | 936 |
| PSMB5_001144932.7 | GGCGGAACTGTTAAGATCAG | 937 |
| PSMB5_001144932.95 | GGCTCCACACCTTGAAGGCC | 938 |
| PSMB5_00130725.41 | GGCTCGACGGGCCAGATCAT | 939 |
| PSMB5_00130725.75 | GGCTGGGATAAGAGAGGCCC | 940 |
| PSMB5_00130725.42 | GGCTTGGTAGATGGCTCGAC | 941 |
| PSMB5_001144932.37 | GGGCTGGCTCCACACCTTGA | 942 |
| PSMB5_001144932.67 | GGTCCAGGGAGTCTCAGTGA | 943 |
| PSMB5_001144932.30 | GGTCTGAGCCTGGCCGCGCC | 944 |
| PSMB5_00130725.51 | GGTGTATCAGTACAAAGGCA | 945 |
| PSMB5_00130725.27 | GGTTGCAGCTTAACTCACCA | 946 |
| PSMB5_001144932.41 | GTAAGCACCCGCTGTAGCCC | 947 |
| PSMB5_001144932.24 | GTGAACCAGCGCGGGTTTTT | 948 |
| PSMB5_00130725.35 | GTGAAGGGAACCGGATTTCA | 949 |
| PSMB5_00130725.10 | GTGGCTCTACCCCCTGAAAG | 950 |
| PSMB5_00130725.73 | GTGTATCAGTACAAAGGCAT | 951 |
| PSMB5_00130725.58 | GTTGACTGCACCTCCTGAGT | 952 |
| PSMB5_00130725.77 | TAGATCAGCCACATTGTCAC | 953 |
| PSMB5_001144932.20 | TAGCGGTCTCTCCAACACGC | 954 |
| PSMB5_001144932.44 | TATCACCTTCTTCACCGTCT | 955 |
| PSMB5_001144932.40 | TCATAGTTGCAGCTGACTCC | 956 |
| PSMB5_00130725.17 | TCCAGCCATCCTCCCGCACG | 957 |
| PSMB5_00130725.25 | TCCATGGGCACCATGATCTG | 958 |
| PSMB5_00130725.54 | TCGGGGCTATTCCTATGACC | 959 |
| PSMB5_00130725.33 | TCTACTACGTGGACAGTGAA | 960 |
| PSMB5_001144932.81 | TCTCAGTGATGGTCTGAGCC | 961 |
| PSMB5_00130725.53 | TCTGGCTCTGTGTATGCATA | 962 |
| PSMB5_00130725.49 | TCTGGGAACGGCTGTTGGCT | 963 |
| PSMB5_00130725.57 | TCTGTAGGTGGCTTGGTAGA | 964 |
| PSMB5_001144932.31 | TCTTCTGGGACACCCCAGCC | 965 |
| PSMB5_00130725.6 | TGAAGGGAACCGGATTTCAG | 966 |
| PSMB5_001144932.68 | TGAGCCTGGCCGCGCCAGGC | 967 |
| PSMB5_00130725.15 | TGAGTAGGCATCTCTGTAGG | 968 |
| PSMB5_001144932.38 | TGATCTTAACAGTTCCGCCA | 969 |
| PSMB5_00130725.40 | TGCATATGGGGTCATGGATC | 970 |
| PSMB5_00130725.12 | TGCATCCACCCTCTTTCAGG | 971 |
| PSMB5_001144932.43 | TGCCTCCCAGACGGTGAAGA | 972 |
| PSMB5_001144932.58 | TGCTGAGAGGATCGGCAACA | 973 |
| PSMB5_001144932.42 | TGCTTACATTGCCTCCCAGA | 974 |
| PSMB5_001144932.104 | TGCTTGAAACCTAAGTCATT | 975 |
| PSMB5_00130725.45 | TGGCTCTACCCCCTGAAAGA | 976 |
| PSMB5_00130725.38 | TGGCTCTGTGTATGCATATG | 977 |
| PSMB5_00130725.43 | TGGCTTGGTAGATGGCTCGA | 978 |
| PSMB5_001144932.5 | TGGGACACCCCAGCCTGGCG | 979 |
| PSMB5_001144932.80 | TGGGGGTCGTGCAGATCTGC | 980 |
| PSMB5_001144932.82 | TGGGGTGTCCCAGAAGAGCC | 981 |
| PSMB5_00130725.28 | TGGTTGCAGCTTAACTCACC | 982 |
| PSMB5_001144932.57 | TGTGGGTGTGCTGAGAGGAT | 983 |
| PSMB5_00130725.66 | TGTGTATGCATATGGGGTCA | 984 |
| PSMB5_001144932.78 | TGTTTTGTGGGTGTGCTGAG | 985 |
| PSMB5_001144932.105 | TTGGAATGTACTTGTTTTGT | 986 |
| PSMB5_001144932.32 | TTTCGATTCCTGGCTCTTCT | 987 |
| PSMB5_001144932.98 | TTTGGAATGTACTTGTTTTG | 988 |
| PSMB5_00130725.82 | TTTGTACTGATACACCATGT | 989 |

TABLE 6B
safe harbor sgRNA sequences

| sgRNA Name | sgRNA sequence | SEQ ID NO: |
|---|---|---|
| SafeHarbor.1 | GGCTAAATTCCTCTTATTCA | 138 |
| SafeHarbor.2 | GTAACCAAGAGTCAGGACTG | 139 |
| SafeHarbor.3 | GGGATAATATAAGGCATTCT | 140 |
| SafeHarbor.4 | GGATCTTATAATCTAGTTAT | 141 |
| SafeHarbor.5 | GTTAATGCCTTGGTCAAATG | 142 |
| SafeHarbor.6 | GTGTAAACTAAGACCTAAGT | 143 |
| SafeHarbor.7 | GCTAAAGTTGTCATTGATTT | 144 |
| SafeHarbor.8 | GTGCTTCCGACAAACTACAA | 145 |
| SafeHarbor.9 | GGAACGTAGGTAATAAGGTC | 146 |
| SafeHarbor.10 | GATTCTTCATATCTTTCTCA | 147 |
| SafeHarbor.11 | GCTCATGAGACACTTCACAG | 148 |
| SafeHarbor.12 | GTCAGCATTAAACATGCTTA | 149 |
| SafeHarbor.13 | GTGAAAGTTCTCATCTTCTT | 150 |
| SafeHarbor.14 | GCATGAGAAGAGGAGATTGA | 151 |
| SafeHarbor.15 | GACTGTTCATAGGACCCTAA | 152 |
| SafeHarbor.16 | GCCCTGTCTGTATCCAGTCC | 153 |
| SafeHarbor.17 | GGGATCTTTCAGTGTAGGTA | 154 |
| SafeHarbor.18 | GATTCTGTATAATGGAAATC | 155 |
| SafeHarbor.19 | GACATGTCCTAATTGTATGG | 156 |
| SafeHarbor.20 | GTGTGCTTTGAAGAATAATG | 157 |
| SafeHarbor.21 | GCAATATGATCTCATTTGTG | 158 |
| SafeHarbor.22 | GAGTTTAGAGGTTTGAGATT | 159 |
| SafeHarbor.23 | GTGGTCCTGGACTGGTCTCA | 160 |
| SafeHarbor.24 | GTTATGCCAACACATTTGTA | 161 |
| SafeHarbor.25 | GTTACATACAAAAATTGGAT | 162 |
| SafeHarbor.26 | GCATATTATCACTCCAGTGA | 163 |
| SafeHarbor.27 | GACATTGGGATTAAATTTGG | 164 |
| SafeHarbor.28 | GGTGGCCGCCATCATGGCTG | 165 |
| SafeHarbor.29 | GGCAGATCAGAATGTGAGCT | 166 |
| SafeHarbor.30 | GAGGAAGGAGTTATATTGAC | 167 |
| SafeHarbor.31 | GAGCCAAAGATAAGCATGAG | 168 |
| SafeHarbor.32 | GGCTACTCAGATATAGTCAT | 169 |
| SafeHarbor.33 | GTTATTTGATGAGCAGCTAT | 170 |
| SafeHarbor.34 | GACGTAGTAAGGTAGAGACA | 171 |
| SafeHarbor.35 | GTGATGAAGAGTGCTACAGC | 172 |
| SafeHarbor.36 | GCTAGGGACTTCAAAGTTAT | 173 |
| SafeHarbor.37 | GATATCTTCCCAATGATGAC | 174 |
| SafeHarbor.38 | GAGTAGTTTCTGACGTCCGA | 175 |
| SafeHarbor.39 | GAGCATAATGAAGGTTCTTG | 176 |
| SafeHarbor.40 | GCGTTTCCAATCCCAGAGAG | 177 |
| SafeHarbor.41 | GGCCTAATAGCTTTGGTAGA | 178 |
| SafeHarbor.42 | GACAGGAGGAACTTGTAACC | 179 |
| SafeHarbor.43 | GAGAGCACTCAGCAAAATCA | 180 |
| SafeHarbor.44 | GCGTTGGTGAAATTACAATT | 181 |
| SafeHarbor.45 | GTTAATGATCAAAAGTTACA | 182 |
| SafeHarbor.46 | GAGAGAATTGCTATTCTGAG | 183 |
| SafeHarbor.47 | GATTGTATGAAAACATAGAT | 184 |
| SafeHarbor.48 | GGCTACCTGTCTATTGGCAC | 185 |
| SafeHarbor.49 | GGCATGTGTGTCTGAATACA | 186 |
| SafeHarbor.50 | GCTGAAGCTCTGGCAAGAGC | 187 |
| SafeHarbor.51 | GTACCTTAATCACACCTTTG | 188 |
| SafeHarbor.52 | GTTCACATAGCAGTACTTGT | 189 |
| SafeHarbor.53 | GACTGACCTTTCTTTGAGAG | 190 |
| SafeHarbor.54 | GACTTGAATGATCAATTACT | 191 |
| SafeHarbor.55 | GTTCTGAGTTACTGGAACCC | 192 |
| SafeHarbor.56 | GCAAGATCAGGTAAGTATCT | 193 |
| SafeHarbor.57 | GTCGTGAAGCTGTGTTTGAC | 194 |
| SafeHarbor.58 | GGTCTTGAAATAAAATTTAG | 195 |
| SafeHarbor.59 | GACTGCTTCTTAGTTAGGTA | 196 |
| SafeHarbor.60 | GGAAATCCTTGAGTTTCAGG | 197 |
| SafeHarbor.61 | GCCCAAGCAGGCTACATTGC | 198 |
| SafeHarbor.62 | GAGGTGGCAAAGAATGTGCC | 199 |
| SafeHarbor.63 | GTTCAAATAATAGGGTGCAT | 200 |
| SafeHarbor.64 | GAGGGGATACTCAAGCTAGG | 201 |
| SafeHarbor.65 | GGGTATCAGCTCACCTCCTC | 202 |
| SafeHarbor.66 | GAAGTACTGGCAATGCAACT | 203 |
| SafeHarbor.67 | GACATAGCCTGCAATTGTTT | 204 |
| SafeHarbor.68 | GGGCAGATTGGAAGAGCCCT | 205 |
| SafeHarbor.69 | GTGTACAACATCACAGCATA | 206 |
| SafeHarbor.70 | GGGTGGTTCTGAATGGGAGC | 207 |
| SafeHarbor.71 | GCTATCCTTAAATTGGCCTG | 208 |
| SafeHarbor.72 | GCCTGAATATAGTGAAAGTC | 209 |
| SafeHarbor.73 | GGGAAGTCCTGGGGTTTGAT | 210 |
| SafeHarbor.74 | GTCAGTTATTCTTTCCTCTA | 211 |
| SafeHarbor.75 | GCATGGTCACAATAATCTTG | 212 |
| SafeHarbor.76 | GGGAGGATAAGAGACACTTT | 213 |
| SafeHarbor.77 | GCTTATTTAGTTTGGTTCAA | 214 |
| SafeHarbor.78 | GTCTCTACTAGAACTCAATC | 215 |
| SafeHarbor.79 | GGAGCTTGGTATCTAAAATT | 216 |
| SafeHarbor.80 | GATGTTCACTGTTAATTGAT | 217 |
| SafeHarbor.81 | GCTACTTAAATCATTGCCAT | 218 |
| SafeHarbor.82 | GCACTTCACCTGAGAAAAAC | 219 |
| SafeHarbor.83 | GCTTGCTTGTCTCTGTTTCG | 220 |
| SafeHarbor.84 | GTCAACAGCAAGGCTACTGA | 221 |
| SafeHarbor.85 | GACAGAAGAAGCTAGAAGTC | 222 |
| SafeHarbor.86 | GTACAACCCAAAGTATATGG | 223 |
| SafeHarbor.87 | GAATCCCGGGCTTTCTCTGT | 224 |
| SafeHarbor.88 | GATAATTTCAGGAGTGAGAT | 225 |
| SafeHarbor.89 | GTATTGTGATCAAGTAATTT | 226 |
| SafeHarbor.90 | GAACCTAAAAATATAGTTGT | 227 |
| SafeHarbor.91 | GCATTGGTGCCCAGTAGGAG | 228 |
| SafeHarbor.92 | GAATACTGTGAGAAATTTCA | 229 |
| SafeHarbor.93 | GTCAAGATATACCTAGCAAA | 230 |
| SafeHarbor.94 | GACCTCACTTACTGTTGCCA | 231 |
| SafeHarbor.95 | GCATACCATAGGGTAAAGGC | 232 |
| SafeHarbor.96 | GGTGACAATCAAACTGGCAA | 233 |
| SafeHarbor.97 | GGTATTGTCAATGTAAAAAG | 234 |
| SafeHarbor.98 | GCACAGTAAATATACGTGTG | 235 |
| SafeHarbor.99 | GTGTGCCCCTCCAAAAGAGA | 236 |
| SafeHarbor.100 | GACATATGCTATGCAGAGTT | 237 |
| SafeHarbor.101 | GTAAGAATCAAATCATCATG | 238 |
| SafeHarbor.102 | GGAAATTGCTTCTGGTTTAT | 239 |
| SafeHarbor.103 | GTAGATGAGCTCTTATCAGT | 240 |
| SafeHarbor.104 | GGCTTTGTTCATGACTTTGA | 241 |
| SafeHarbor.105 | GCACCAGTCTATGCCACCAC | 242 |
| SafeHarbor.106 | GTAATGACTTGGGGGAGATA | 243 |
| SafeHarbor.107 | GAGTCTGTCTCTAATGAGAC | 244 |
| SafeHarbor.108 | GTGGTCCACAGACAATGCAT | 245 |
| SafeHarbor.109 | GGTTAAGAAAAGACACTCAG | 246 |
| SafeHarbor.110 | GGTAATCATAAGTTGTATAA | 247 |
| SafeHarbor.111 | GGCCCTCCTTAGAAGTTGCA | 248 |
| SafeHarbor.112 | GAAATTGGTCCCCACCTTCA | 249 |
| SafeHarbor.113 | GTCCAAGAACAAAGCAAAGA | 250 |
| SafeHarbor.114 | GATGAGCCAATCTTTAGCAA | 251 |
| SafeHarbor.115 | GTGAATCAAGAAGCAATGTC | 252 |
| SafeHarbor.116 | GAAAGGCAGACATGGCTAAA | 253 |
| SafeHarbor.117 | GACAAAAGCAGAATACCAGA | 254 |
| SafeHarbor.118 | GCACACAAAATATCGTTATT | 255 |
| SafeHarbor.119 | GAGAAAGGCCCAGCTCTGAT | 256 |
| SafeHarbor.120 | GCCAGTCTACCCACTGTCCC | 257 |
| SafeHarbor.121 | GCAGGGTGAAGGTCCTCCTC | 258 |
| SafeHarbor.122 | GAAGAGACTACAATTATTCT | 259 |
| SafeHarbor.123 | GATATCCTTTGTGTTAACTT | 260 |
| SafeHarbor.124 | GAATGACTCGCATGACTTTA | 261 |
| SafeHarbor.125 | GGATGTTCAAACCTTCAAAA | 262 |
| SafeHarbor.126 | GAGAATATATGTTTCCATTA | 263 |
| SafeHarbor.127 | GGAAAAGTAATGAATCATAC | 264 |
| SafeHarbor.128 | GTTACACGAAGCACAGGGTG | 265 |
| SafeHarbor.129 | GAACTAGGTGCTCAAGGAAT | 266 |
| SafeHarbor.130 | GGCAAAGACCAGTCTGATAC | 267 |
| SafeHarbor.131 | GTCTAGTTTCACAATAATTT | 268 |
| SafeHarbor.132 | GCTTTATATAAGATATGAGA | 269 |
| SafeHarbor.133 | GCATAGGATATTATATTTCG | 270 |
| SafeHarbor.134 | GACCTTGACTGCTCCTGAAC | 271 |
| SafeHarbor.135 | GCAGCTCCCTAGTTCACAGA | 272 |
| SafeHarbor.136 | GTCTGACCAGAGGTGGAGAG | 273 |
| SafeHarbor.137 | GAATCACATTGTACCACAAA | 274 |
| SafeHarbor.138 | GACAAAATTGATACAACAGC | 275 |
| SafeHarbor.139 | GAATTCCAAGACTTCACATT | 276 |
| SafeHarbor.140 | GACAGGGACCGCCATCCACT | 277 |
| SafeHarbor.141 | GTTGTATGGTTCCTAAGGAT | 278 |
| SafeHarbor.142 | GAATATCCACTACTAGCTTT | 279 |
| SafeHarbor.143 | GCCATTAATCATGATCTGGA | 280 |
| SafeHarbor.144 | GGTGAATAGGTAGGTATTGA | 281 |
| SafeHarbor.145 | GCTCATCAAAGGTAGTAAAC | 282 |
| SafeHarbor.146 | GGGACCCAGCCCTTGGGCTG | 283 |
| SafeHarbor.147 | GTGCACCTTTCTATAAATGT | 284 |
| SafeHarbor.148 | GACTTCATTAAAAGCAGTCT | 285 |
| SafeHarbor.149 | GTTGAACTTGTGAACACAAA | 286 |
| SafeHarbor.150 | GGGTCCTCACCAGGAAATTT | 287 |
| SafeHarbor.151 | GTAGCCTATTGGCAATTGGC | 288 |
| SafeHarbor.152 | GCATAAATAAAATCGATTCC | 289 |
| SafeHarbor.153 | GAAGGGCAATAATTGGTACA | 290 |
| SafeHarbor.154 | GAGTTCTTAATAACATTCTA | 291 |
| SafeHarbor.155 | GCTTTCTACTTGCCTTAGAT | 292 |
| SafeHarbor.156 | GCTTCTTATTTCTCTCCAGT | 293 |
| SafeHarbor.157 | GCATTCTGTCCTAATAAGAA | 294 |
| SafeHarbor.158 | GCTTAAGCTAGTTTAAAGAA | 295 |
| SafeHarbor.159 | GGTTTCCAGTGTTTATCTGT | 296 |
| SafeHarbor.160 | GAGAGTCTAGGTACGTTCTC | 297 |
| SafeHarbor.161 | GCTTTCAAGTTAACATAGCT | 298 |
| SafeHarbor.162 | GTAAAATGAACCGAGCTTTA | 299 |
| SafeHarbor.163 | GTAAGATTATTAACCCCTTC | 300 |
| SafeHarbor.164 | GGGTCCTCACGATAGAAGAA | 301 |
| SafeHarbor.165 | GATTACACTCAAGAAAGCGA | 302 |
| SafeHarbor.166 | GATGTAGACGTAGAAGTGAT | 303 |
| SafeHarbor.167 | GTGAGTTACAGAAATTAGCA | 304 |
| SafeHarbor.168 | GCAGGGGGACACGGGCACAT | 305 |
| SafeHarbor.169 | GACAATTGTGTTGCAGACAA | 306 |
| SafeHarbor.170 | GTCAATGGGAAATTATAAAC | 307 |
| SafeHarbor.171 | GAGTTATAGCACACTTAGAA | 308 |
| SafeHarbor.172 | GATTGAAACCAGAAAATAAG | 309 |
| SafeHarbor.173 | GGAGTCTAGTGATAGGGGTA | 310 |
| SafeHarbor.174 | GGGATAGTCTTAGAAGGCTT | 311 |
| SafeHarbor.175 | GTCAATTGATTCACTGGAAT | 312 |
| SafeHarbor.176 | GTATTCCTGCAAGATAATTC | 313 |
| SafeHarbor.177 | GGTCAAGCAACAGGCATAAT | 314 |
| SafeHarbor.178 | GACATCCATAACTTCCTAAC | 315 |
| SafeHarbor.179 | GTCAAACAAAAGCGTCTATA | 316 |
| SafeHarbor.180 | GCTAGATTAATATGAATGAG | 317 |
| SafeHarbor.181 | GAACCCCATAGGAGGTTTAG | 318 |
| SafeHarbor.182 | GCCTCTTTCCCCTGCCGGCA | 319 |
| SafeHarbor.183 | GGTAAGGGCTGCTTATCTTT | 320 |
| SafeHarbor.184 | GTATTCAGTATAATCAAGGA | 321 |
| SafeHarbor.185 | GTTGTCTTATGGGACTGCAT | 322 |
| SafeHarbor.186 | GTATACGATATGATTGACTC | 323 |
| SafeHarbor.187 | GGTAGAGACAAAATATATTT | 324 |
| SafeHarbor.188 | GTACCTATGTCCTTGAGGCT | 325 |
| SafeHarbor.189 | GGCAAAAGAACGTCTGTAAT | 326 |
| SafeHarbor.190 | GGACTAGTTTACCTAGGGAG | 327 |
| SafeHarbor.191 | GGAGGGTGGAGCAAAGAAAG | 328 |
| SafeHarbor.192 | GAGCCATATTATGTCCTTTA | 329 |
| SafeHarbor.193 | GTGCACTCTATGCACCAAAG | 330 |
| SafeHarbor.194 | GGTCTCCCGAGTCATTGTTG | 331 |
| SafeHarbor.195 | GCAATCATTCTGGTTCAGGC | 332 |
| SafeHarbor.196 | GCACAGGTTCCCCTCCTAAC | 333 |
| SafeHarbor.197 | GATCAGGGAATCTTTGAGAA | 334 |
| SafeHarbor.198 | GAACCCAGCTGTCCTCGCTG | 335 |
| SafeHarbor.199 | GCTAACTGTGTTACAAGCAG | 336 |
| SafeHarbor.200 | GTGATCAAAGAGAGAGGTGT | 337 |
| SafeHarbor.201 | GGAAAGCCCGTTGTATTTAT | 338 |
| SafeHarbor.202 | GGTCCCCCACTTTCTCCTTG | 339 |
| SafeHarbor.203 | GCCAGATGACCATAGAAACT | 340 |
| SafeHarbor.204 | GGTGCAATCCAAAGGTGGGC | 341 |
| SafeHarbor.205 | GTGTAAAATCACTTTAAACT | 342 |
| SafeHarbor.206 | GTCACATGTTCAAGTTTAAC | 343 |
| SafeHarbor.207 | GAAGCTTAGTCCTGAATTGT | 344 |
| SafeHarbor.208 | GGGTCTGTTTCCTTGTGTTA | 345 |
| SafeHarbor.209 | GATAGAGACTGGATGAAGTT | 346 |
| SafeHarbor.210 | GCAACAAGGCAAATGTGGTA | 347 |
| SafeHarbor.211 | GCTATTTAGCTCAACCTTGT | 348 |
| SafeHarbor.212 | GTGCCATTATCATTTCCTCA | 349 |
| SafeHarbor.213 | GCAAATAGAAGAGACAATCT | 350 |
| SafeHarbor.214 | GAAAATATATGGACTGGGAT | 351 |
| SafeHarbor.215 | GAATAGAACTCCTGCCATCA | 352 |
| SafeHarbor.216 | GCTTTCTACCTGGATGTTTA | 353 |
| SafeHarbor.217 | GCTAACTTGAGGGCAAAAGA | 354 |
| SafeHarbor.218 | GTGGTAAAAATGTGCTTTGT | 355 |
| SafeHarbor.219 | GAGCCTCAGCTGGTGCATGG | 356 |
| SafeHarbor.220 | GCCTATGCCGCAATACCCTC | 357 |
| SafeHarbor.221 | GACCTGTGTAAACCAGCTAA | 358 |
| SafeHarbor.222 | GACCTCATTCCTGAGTGTGT | 359 |
| SafeHarbor.223 | GTGTTTGCCTCATAATAACC | 360 |
| SafeHarbor.224 | GACTGGGCATACAGCCATTT | 361 |
| SafeHarbor.225 | GGCATACTACATTGGCTTTA | 362 |
| SafeHarbor.226 | GCAAACATATTGGAGTACTG | 363 |
| SafeHarbor.227 | GGGGAGTAGGGAAGAGCTTA | 364 |
| SafeHarbor.228 | GGGCTCGTATGTCGTTCTTC | 365 |
| SafeHarbor.229 | GTGCCTTATCTATTTCCACA | 366 |
| SafeHarbor.230 | GGTAATTACCTGCTCTCTGC | 367 |
| SafeHarbor.231 | GTCTGATAACTTGTGTTACT | 368 |
| SafeHarbor.232 | GACTGAGTTAATAATAGCGG | 369 |
| SafeHarbor.233 | GAATATTGTGCACTGTATTT | 370 |
| SafeHarbor.234 | GTTTCTAAATGTGATCTGTG | 371 |
| SafeHarbor.235 | GCACACTGGCTAGTTAAGGA | 372 |
| SafeHarbor.236 | GGAGGAGTGTGCAATGAAGC | 373 |
| SafeHarbor.237 | GAGGACGGGTGGGAAGTTAG | 374 |
| SafeHarbor.238 | GATACTGTAGCAGTTACTGA | 375 |
| SafeHarbor.239 | GATTCTAAGCAAAGGACAGA | 376 |
| SafeHarbor.240 | GGAGCTTAGACCATATTTGG | 377 |
| SafeHarbor.241 | GTGTCCGTGGGTCTGTTCCC | 378 |
| SafeHarbor.242 | GCAATAGCTGTGAGCTCATA | 379 |
| SafeHarbor.243 | GGGATGGGCCATCCAGCTGT | 380 |
| SafeHarbor.244 | GACAGATTACTTAATAAAAG | 381 |
| SafeHarbor.245 | GTGGCAAGGTTAAGTACAAT | 382 |
| SafeHarbor.246 | GGAGGAAACAGAATAATGGC | 383 |
| SafeHarbor.247 | GTGAATTAATGTCATTTCAC | 384 |
| SafeHarbor.248 | GTGAACTAGAACACTGAGAG | 385 |
| SafeHarbor.249 | GATGCTGTGGCCAATGTGCA | 386 |
| SafeHarbor.250 | GACTGTAAGCATTCCTGACA | 387 |
| SafeHarbor.251 | GTCCTAATTCCATGCCTAAA | 388 |
| SafeHarbor.252 | GTGGGTTCGTTGTCTACTAC | 389 |
| SafeHarbor.253 | GAGACTATTAGATCGTATGT | 390 |
| SafeHarbor.254 | GGTGTAGTATCAAAAATTGA | 391 |
| SafeHarbor.255 | GATAGCTCTTAAGGATAAAT | 392 |
| SafeHarbor.256 | GATTCAGTCACATCACAATA | 393 |
| SafeHarbor.257 | GTCTAAGAAAGACTTCTAGG | 394 |
| SafeHarbor.258 | GATTTGGGTCTTTGCGCATC | 395 |
| SafeHarbor.259 | GACCTTAAAGTTATAGTTAA | 396 |
| SafeHarbor.260 | GCTCTGCATCTTTCCCCAGG | 397 |
| SafeHarbor.261 | GACCTAAGTTTGAGAATGAG | 398 |
| SafeHarbor.262 | GAAAGTACATTCATTAGCAT | 399 |
| SafeHarbor.263 | GGAGAACGTGGTGATAAAGC | 400 |
| SafeHarbor.264 | GGCAACATGGCAAAATAGTT | 401 |
| SafeHarbor.265 | GATAATAGCAGAGAGAGGTG | 402 |
| SafeHarbor.266 | GGACTTTAAGGAATTCAGCT | 403 |
| SafeHarbor.267 | GAATATTGGGGGGTGGATGG | 404 |
| SafeHarbor.268 | GGAGTAAGTATGTGTGTTGA | 405 |
| SafeHarbor.269 | GTATTGGATAAGGGAGCTCA | 406 |
| SafeHarbor.270 | GTGAGTTGGGAGATGTACTG | 407 |
| SafeHarbor.271 | GTTTACAATTTCATTTGTAC | 408 |
| SafeHarbor.272 | GTCCATTCAATTTGGACATG | 409 |
| SafeHarbor.273 | GAGTGCTTACTGGGAATGAG | 410 |
| SafeHarbor.274 | GCTAATTGTTCAAAAAGCCC | 411 |
| SafeHarbor.275 | GCTTTCAAGAGTTTATTTGA | 412 |
| SafeHarbor.276 | GATATTCTGTGCAATCTGTT | 413 |
| SafeHarbor.277 | GTGTAGGACTACGCTGGCAC | 414 |
| SafeHarbor.278 | GTCTTAAAGAGTAAAGTACA | 415 |
| SafeHarbor.279 | GTTAGACTGCAAACACCCAC | 416 |
| SafeHarbor.280 | GCCTAGGAGAAGCCCTGGCA | 417 |
| SafeHarbor.281 | GTCGAGTATTTCTAATCTTT | 418 |
| SafeHarbor.282 | GAATCTGAGACATCATTCAT | 419 |
| SafeHarbor.283 | GACAAAAGATTATGCTTCCC | 420 |
| SafeHarbor.284 | GAGAATTACATTCATGATCT | 421 |
| SafeHarbor.285 | GAACTGAGCTTCTACCATGC | 422 |
| SafeHarbor.286 | GGTAAGATTGTAATAGCTTG | 423 |
| SafeHarbor.287 | GTCAGAAATGATCTCGTCCT | 424 |
| SafeHarbor.288 | GACATATCTAAGAACTGAGC | 425 |
| SafeHarbor.289 | GCTTCAATATGACAGAACTC | 426 |
| SafeHarbor.290 | GGAGAGCAAATCAGCATATC | 427 |
| SafeHarbor.291 | GCAAAATAGCCGCACAGAAA | 428 |
| SafeHarbor.292 | GCATATTTCTATACAATACA | 429 |
| SafeHarbor.293 | GATGCAAATTCATGGTGGTA | 430 |
| SafeHarbor.294 | GAACTGTAATAGTCTTGAGC | 431 |
| SafeHarbor.295 | GAACTCACTACATTAAGGCT | 432 |
| SafeHarbor.296 | GAGGTAAATCAGTACAAACA | 433 |
| SafeHarbor.297 | GTTGTTTCTAAGATTAAAAG | 434 |
| SafeHarbor.298 | GTGGTAGTCAGTTTCACAAA | 435 |
| SafeHarbor.299 | GGTTTCAAATAGTTGGATCA | 436 |
| SafeHarbor.300 | GAATATGAAAGACATCATAA | 437 |
| SafeHarbor.301 | GAAGTAGGAAGGAGATTGCC | 438 |
| SafeHarbor.302 | GGAAAAGTGCTGTTTGCATT | 439 |
| SafeHarbor.303 | GAGCATTAGGCTGGGGCCTT | 440 |
| SafeHarbor.304 | GTCTAGGTATGATTAGAAGA | 441 |
| SafeHarbor.305 | GAGTTATAATCTTCAGAAAA | 442 |
| SafeHarbor.306 | GCTGTAATGAGACTTCAGCT | 443 |
| SafeHarbor.307 | GTGTGCAATCTGAAGGAAAT | 444 |
| SafeHarbor.308 | GTGATGAGGTCGCTGAAGTT | 445 |
| SafeHarbor.309 | GTGGAGCCCTTATAACCCTG | 446 |
| SafeHarbor.310 | GTTGGATTATTTCTTCTATA | 447 |
| SafeHarbor.311 | GGATTTCTACATTATATACT | 448 |
| SafeHarbor.312 | GCTAATGTAGATCAAGTTAT | 449 |
| SafeHarbor.313 | GATTGCAAGAGACTGAACTC | 450 |
| SafeHarbor.314 | GGGTGAACTTGAGTGAACTT | 451 |
| SafeHarbor.315 | GGGCTCAAATCCCTATAATT | 452 |
| SafeHarbor.316 | GATAGAAGGTATTAACTCCC | 453 |
| SafeHarbor.317 | GGCTATAAGCACAAATGTAA | 454 |
| SafeHarbor.318 | GATTCCCATTGCATGCCAGT | 455 |
| SafeHarbor.319 | GCAAATTACAATTATGTTTC | 456 |
| SafeHarbor.320 | GAATTAAATTCACTTTGAAC | 457 |
| SafeHarbor.321 | GAGCAGACAGGAAATAAAGC | 458 |
| SafeHarbor.322 | GCCCACCAGTCCTTCTCACT | 459 |
| SafeHarbor.323 | GTTAAGAAGTGAAAGAAATT | 460 |
| SafeHarbor.324 | GTTGAATTGAATGGGTCATT | 461 |
| SafeHarbor.325 | GTAGACACAAACTTGTGTAA | 462 |
| SafeHarbor.326 | GAGCGTACTATATTCTTAAA | 463 |
| SafeHarbor.327 | GGTGGTACATCGTTGAAGGA | 464 |
| SafeHarbor.328 | GATGAACTCCCAATCACAGG | 465 |
| SafeHarbor.329 | GTATAAATAAGGATAAGGTA | 466 |
| SafeHarbor.330 | GGAAATAATCTTGGAACATA | 467 |
| SafeHarbor.331 | GGTAGTTAATCTTCTACTTT | 468 |
| SafeHarbor.332 | GAGAAGAGAACATTCTAGTT | 469 |
| SafeHarbor.333 | GTCGGAGCTCAGTGTTGCAT | 470 |
| SafeHarbor.334 | GAAGAGACATGTTTCAGTGA | 471 |
| SafeHarbor.335 | GTCATATCTGACTTAAATTG | 472 |
| SafeHarbor.336 | GGAGAATATGCTAAAAGCGT | 473 |
| SafeHarbor.337 | GATTGTTGTAGTAGAATAAA | 474 |
| SafeHarbor.338 | GTAAGCAGCACCACCACTTA | 475 |
| SafeHarbor.339 | GTCTTGTGCTGACATGCTCA | 476 |
| SafeHarbor.340 | GCAGACTTTATTAGCTAGTG | 477 |
| SafeHarbor.341 | GAGGTATTTGATATGACTCA | 478 |
| SafeHarbor.342 | GCAGGTTGCCCATTCTCCCA | 479 |
| SafeHarbor.343 | GAGGGGACGTTGACCTGTGG | 480 |
| SafeHarbor.344 | GAACCCAAGGATTTATAAAG | 481 |
| SafeHarbor.345 | GTGTTCAGGACATGTACTCA | 482 |
| SafeHarbor.346 | GGTGATGATAGTCAAATACC | 483 |
| SafeHarbor.347 | GCTTTACAGCTAATTTCTAA | 484 |
| SafeHarbor.348 | GGTATCTACATTAACACTCA | 485 |
| SafeHarbor.349 | GACAGTTTGCTTACTATGGA | 486 |
| SafeHarbor.350 | GAAAAACTCTTAGCTTAATG | 487 |
| SafeHarbor.351 | GTCATCTTAACTTCAGTAGA | 488 |
| SafeHarbor.352 | GATCACTGGTAGGCCACAGT | 489 |
| SafeHarbor.353 | GAGAAAGGCAAGTGCATCAA | 490 |
| SafeHarbor.354 | GAACTGATAAAGATTCAGTA | 491 |
| SafeHarbor.355 | GCCATTCAAAAGCAGCTATA | 492 |
| SafeHarbor.356 | GACAGAACTTCTTTGAGCTA | 493 |
| SafeHarbor.357 | GGGTGACATTGAAATTTAAC | 494 |
| SafeHarbor.358 | GACTATAAACTGCACACTAT | 495 |
| SafeHarbor.359 | GCTATGGTGGGAAAGCTCAT | 496 |
| SafeHarbor.360 | GACTAACTTGCTAATGGCTA | 497 |
| SafeHarbor.361 | GAGAGTCACTTCAAAGTGTG | 498 |
| SafeHarbor.362 | GAGTGTATTTGTGGACAATA | 499 |
| SafeHarbor.363 | GAAGAATTAGGGTTCCATTT | 500 |
| SafeHarbor.364 | GAGGAGTGGCACTTTATACT | 501 |
| SafeHarbor.365 | GAAGGATGCAGTAGCCATTG | 502 |
| SafeHarbor.366 | GTGCATTGTTGGTGGTTGTG | 503 |
| SafeHarbor.367 | GAGAAGTTATGCAAATTTAT | 504 |
| SafeHarbor.368 | GAAATAGATTGGCAGAGTGT | 505 |
| SafeHarbor.369 | GTGGGGTGGGCTCCCTGCCT | 506 |
| SafeHarbor.370 | GTCTCTAACAAGACTGAAAT | 507 |
| SafeHarbor.371 | GCAGAGTAGATCTACATCTT | 508 |
| SafeHarbor.372 | GTGCCAGCTAAGATGAAATT | 509 |
| SafeHarbor.373 | GATGGTGATGCACCAACTTT | 510 |
| SafeHarbor.374 | GAAGTGTTGCCATTCAATTC | 511 |
| SafeHarbor.375 | GAGAGAGTTGGAATAAGCTA | 512 |
| SafeHarbor.376 | GAGGGTACTTATTTCAACTT | 513 |
| SafeHarbor.377 | GCTACATGTTCTAGAATACA | 514 |
| SafeHarbor.378 | GAGAAATCTCTTTGAGCTGG | 515 |
| SafeHarbor.379 | GGCTTTGTGTCTGACTTTCC | 516 |
| SafeHarbor.380 | GGATTAGATCAATTATTCTA | 517 |
| SafeHarbor.381 | GATTCTGGAAATAAGTACCT | 518 |
| SafeHarbor.382 | GAGATAAAATTGCGAGACCA | 519 |
| SafeHarbor.383 | GACAAAATTTAGCAACTCAG | 520 |
| SafeHarbor.384 | GCAGATACTCACCATTACCC | 521 |
| SafeHarbor.385 | GGTGATTGTTGCAGCTGTCA | 522 |
| SafeHarbor.386 | GATAGACTTGTGAAGGAAAC | 523 |
| SafeHarbor.387 | GAGTCACTGGATTGTTGTCC | 524 |
| SafeHarbor.388 | GGATTATATGGGAGGTACAC | 525 |
| SafeHarbor.389 | GCTTAAAAATACTATCTGCT | 526 |
| SafeHarbor.390 | GACAAGGAGGACCAAAGTTG | 527 |
| SafeHarbor.391 | GGCAGTGATTTACTCCTATC | 528 |
| SafeHarbor.392 | GATCTTCCAGGACTGTTAGA | 529 |
| SafeHarbor.393 | GAAACAAGCTAATATTATCA | 530 |
| SafeHarbor.394 | GTCAGTCTTTACAAATCACT | 531 |
| SafeHarbor.395 | GGCAGTTGAGTAAACGTAAG | 532 |
| SafeHarbor.396 | GCCTCTACTGCTAACTCTAT | 533 |
| SafeHarbor.397 | GTTGTAATTTAAAGCACTCA | 534 |
| SafeHarbor.398 | GCATAAAGAGAACAAGCAAT | 535 |
| SafeHarbor.399 | GGTAGTTGGTCTAATCAGTA | 536 |
| SafeHarbor.400 | GGCTAACACCTGCCAACTTT | 537 |
| SafeHarbor.401 | GTCTAATCTAGCATCAAACT | 538 |
| SafeHarbor.402 | GAGAGAGACTATTTCAGGAT | 539 |
| SafeHarbor.403 | GACCTAGACCAAGCTACGAA | 540 |
| SafeHarbor.404 | GTTACTGATACCAGTCCCTG | 541 |
| SafeHarbor.405 | GCCCTACTGTGGTAACTTTG | 542 |
| SafeHarbor.406 | GTGTAAAGGAATCTTAGCTT | 543 |
| SafeHarbor.407 | GGTGAGACTATTATATTTAT | 544 |
| SafeHarbor.408 | GCTTCAGAGAACTATTTGGT | 545 |
| SafeHarbor.409 | GATGTGTTCGTTGAGGCATA | 546 |
| SafeHarbor.410 | GTTGACTCTAACTATAGAGT | 547 |
| SafeHarbor.411 | GGACAGCCATTGAAGATATG | 548 |
| SafeHarbor.412 | GATGGAGAGCCTGGAGCATA | 549 |
| SafeHarbor.413 | GCATGATTAAAGGTGAGCAT | 550 |
| SafeHarbor.414 | GGAACCCACAGATATAGCTA | 551 |
| SafeHarbor.415 | GCATAGCTTCAGAGTTCAGA | 552 |
| SafeHarbor.416 | GAGAAAAGACGTGTATTTCC | 553 |
| SafeHarbor.417 | GCTAGAGCTTCCTTATGTTT | 554 |
| SafeHarbor.418 | GATGGGCAGTCAGGACTACG | 555 |
| SafeHarbor.419 | GTTCTGCATGAGAAGCACTA | 556 |
| SafeHarbor.420 | GACTCCACCTATCTCAAAAT | 557 |
| SafeHarbor.421 | GATATTTGACAGTGGATAAA | 558 |
| SafeHarbor.422 | GAAAGATTATGGATCATAGT | 559 |
| SafeHarbor.423 | GCATCAATGTACACTGTGGC | 560 |
| SafeHarbor.424 | GCAGCAAGCTATGGTCCATG | 561 |
| SafeHarbor.425 | GGTTGTTTGAATTAAAGACT | 562 |
| SafeHarbor.426 | GAACCCCTGGCTAGTTTCCC | 563 |
| SafeHarbor.427 | GGATAAAGAGTGAACCTGTA | 564 |
| SafeHarbor.428 | GTAGATTTCACTAAATTGTT | 565 |
| SafeHarbor.429 | GTGTAGTTAGAATAAGAAGG | 566 |
| SafeHarbor.430 | GTGGCAATGTCCTGGAGAAA | 567 |
| SafeHarbor.431 | GTGAAGTGCTTTATCTGTAC | 568 |
| SafeHarbor.432 | GAGTTTATATAGGTATGAAA | 569 |
| SafeHarbor.433 | GACCTCATAAACAAATCACT | 570 |
| SafeHarbor.434 | GAAACGTCTGTATGCAAAGC | 571 |
| SafeHarbor.435 | GGTGTGGTGCAAGGGTGAGT | 572 |
| SafeHarbor.436 | GAGAATCTGCTATTGCCAAT | 573 |
| SafeHarbor.437 | GTACTAAGTATCTTGAAATG | 574 |
| SafeHarbor.438 | GTCATGACATGAGTTGCATG | 575 |
| SafeHarbor.439 | GCAGTGATCAGAGACAGTTG | 576 |
| SafeHarbor.440 | GGCAAAATAACTTCATCTAT | 577 |
| SafeHarbor.441 | GCCTGGCCTTCTGTGGAATT | 578 |
| SafeHarbor.442 | GGTGGCCTTTGTTTGCAGGC | 579 |
| SafeHarbor.443 | GAGATGGTATATTTGTCAGA | 580 |
| SafeHarbor.444 | GGGACACCCAGCATCTCAAC | 581 |
| SafeHarbor.445 | GTATATGACAGTAGGGTTGG | 582 |
| SafeHarbor.446 | GGACCCCAGAACTGAAATCA | 583 |
| SafeHarbor.447 | GGGCACCACTGAGAATGTAT | 584 |
| SafeHarbor.448 | GGGACTACAAATATGAAAAA | 585 |
| SafeHarbor.449 | GTAAAATTATGAGCTCCAGT | 586 |
| SafeHarbor.450 | GATTGTGAGTGATGAGAATC | 587 |
| SafeHarbor.451 | GAGACTGAGGGTTGCTCTTA | 588 |
| SafeHarbor.452 | GCATAGAGTGAACACTTTGG | 589 |
| SafeHarbor.453 | GAAGTTCTCCTTTAACCAAT | 590 |
| SafeHarbor.454 | GACCTTGACCAAAGATATTA | 591 |
| SafeHarbor.455 | GTGTGGGCAAGAGACAGTCC | 592 |
| SafeHarbor.456 | GTTGGGGGCTCTCTTGCCAC | 593 |
| SafeHarbor.457 | GGATAAAACTCTAACAGAAC | 594 |
| SafeHarbor.458 | GGAAACATATTACCCCTCCA | 595 |
| SafeHarbor.459 | GCACTATTACTCCACTGAGA | 596 |
| SafeHarbor.460 | GTGAGCAGAGATCACCTTAG | 597 |
| SafeHarbor.461 | GGGTTCATATAGGTCGGAAT | 598 |
| SafeHarbor.462 | GTGCCCCCGATTCTTCCATG | 599 |
| SafeHarbor.463 | GGAACAAAATTTGCACATAA | 600 |
| SafeHarbor.464 | GAGAAAGTCCAAGGGTAAAA | 601 |
| SafeHarbor.465 | GCAATTAACTCTACAAGGAA | 602 |
| SafeHarbor.466 | GTTTCAACCATTAGGGGGCT | 603 |
| SafeHarbor.467 | GGCAGGGGTAGTAAGCTTAG | 604 |
| SafeHarbor.468 | GTACACATCTTCCCAATCAG | 605 |
| SafeHarbor.469 | GTTACTTGGAAAAATGACCA | 606 |
| SafeHarbor.470 | GTACCCGGTAAATCATAGAG | 607 |
| SafeHarbor.471 | GTGTATTATCCTGCATTCCA | 608 |
| SafeHarbor.472 | GGGTAAAACAAATGCATCAT | 609 |
| SafeHarbor.473 | GTGTGTTGGCCTAGGGATGA | 610 |
| SafeHarbor.474 | GGTGTGATAAAACCTCAGAG | 611 |
| SafeHarbor.475 | GAGCTAATTGGTCAGATTCT | 612 |
| SafeHarbor.476 | GTACCAGAGTACAGTGTCCG | 613 |
| SafeHarbor.477 | GGTCAGTGCTCTATCATTTA | 614 |
| SafeHarbor.478 | GTTGCCTATCTTCAGAGTAC | 615 |
| SafeHarbor.479 | GAAGATGCATGGACCTACCA | 616 |
| SafeHarbor.480 | GAATAGACACTGGTTCTCTG | 617 |
| SafeHarbor.481 | GTCAGCTCTTAACATCTGGT | 618 |
| SafeHarbor.482 | GATAACAAGGCTCAGAAGGC | 619 |
| SafeHarbor.483 | GTCAAAACACAGTGAGCTGT | 620 |
| SafeHarbor.484 | GAGAATATAGCTGAAGGTGG | 621 |
| SafeHarbor.485 | GGGATTGACCATCAATACAG | 622 |
| SafeHarbor.486 | GAAACCCCCATCTCAGTCTT | 623 |
| SafeHarbor.487 | GTACAGATACCACTATTTGG | 624 |
| SafeHarbor.488 | GAGTAGCTAGAGGCACTCTT | 625 |
| SafeHarbor.489 | GAGATTTGCAGTGCATGAAT | 626 |
| SafeHarbor.490 | GTTCAACTAAAGGTCTTATG | 627 |
| SafeHarbor.491 | GTGTTTCACTGTTCTCTTCA | 628 |
| SafeHarbor.492 | GTGAAGTAGAGATTATGTAA | 629 |
| SafeHarbor.493 | GTCAAACCAAGTTGAATTCA | 630 |
| SafeHarbor.494 | GATGCTAAAAATCTAAACCT | 631 |
| SafeHarbor.495 | GGCCCTTATTACCAGATTTG | 632 |
| SafeHarbor.496 | GTGGAGATTTGCTTACGAGC | 633 |
| SafeHarbor.497 | GAACCTTGGAGAATTGAATA | 634 |
| SafeHarbor.498 | GATAGAAAAGAGCAGCTACA | 635 |
| SafeHarbor.499 | GCAAGAAGAAACTGCTATTA | 636 |
| SafeHarbor.500 | GTAATGTTGCCGAAGCAATT | 637 |
| SafeHarbor.501 | GAATTTCATTACAGGAAGTA | 638 |
| SafeHarbor.502 | GAAAACACACCTTATCACAG | 639 |
| SafeHarbor.503 | GTTATCTTTGAGAGAACATT | 640 |
| SafeHarbor.504 | GAACTCTTAAGGTTAATAAG | 641 |
| SafeHarbor.505 | GAACCATCCATCCTCACCTG | 642 |
| SafeHarbor.506 | GGAGATGCACTGGTAAAAAG | 643 |
| SafeHarbor.507 | GCTCATCTCCACAGCCATCC | 644 |
| SafeHarbor.508 | GAGTGGCCGGTGCCATTTCT | 645 |
| SafeHarbor.509 | GCTACTAGCGAAGAAGAAGG | 646 |
| SafeHarbor.510 | GTAAGCTTAAAACATTAGTA | 647 |
| SafeHarbor.511 | GTTTACAGGAAGGAGAAGGA | 648 |
| SafeHarbor.512 | GTAATATTTGAGGTATGAAT | 649 |
| SafeHarbor.513 | GATGGCTCACACTTGCTGTA | 650 |
| SafeHarbor.514 | GAAACTGGGAACAAGCTTTA | 651 |
| SafeHarbor.515 | GCTAATGCTTTGCCTACCCC | 652 |
| SafeHarbor.516 | GCCTTACCCTCAGTAGTGAA | 653 |
| SafeHarbor.517 | GAACTGAAGTTTAGAAGTAA | 654 |
| SafeHarbor.518 | GAAATATCATGATGGTGAAG | 655 |
| SafeHarbor.519 | GTGTTGATTCTGAACAAGTT | 656 |
| SafeHarbor.520 | GGCCCTGTCCTGGACATAAA | 657 |
| SafeHarbor.521 | GCACATTCTAATTTGTGGAT | 658 |
| SafeHarbor.522 | GAAGTTAACATGGAATTAAA | 659 |
| SafeHarbor.523 | GTCCTTAGGCTTGCAATGCT | 660 |
| SafeHarbor.524 | GAGAGACAATTTGGGTCTAG | 661 |
| SafeHarbor.525 | GTTAAATCCAATGGATTCCT | 662 |
| SafeHarbor.526 | GTTCTCAATTTACTGGGATT | 663 |
| SafeHarbor.527 | GCAGCTGTGCTCAAAAGACC | 664 |
| SafeHarbor.528 | GAGGCTTAGTTGTAATAATG | 665 |
| SafeHarbor.529 | GCCCCTCAATTCCAGTGTAA | 666 |
| SafeHarbor.530 | GACTGGCAAATACAATTTGC | 667 |
| SafeHarbor.531 | GAATGCAATATAGTGATCTT | 668 |
| SafeHarbor.532 | GGAGAGGGTGGTTTAAAAGC | 669 |
| SafeHarbor.533 | GGGTATACCTTAGGAAAGCT | 670 |
| SafeHarbor.534 | GATGCATTCAATAGCTCTGT | 671 |
| SafeHarbor.535 | GGGCTAAATAAAGCAATGTT | 672 |
| SafeHarbor.536 | GTTATTCATAAATTGTAAGC | 673 |
| SafeHarbor.537 | GTGACATAGTGGGATAGCCC | 674 |
| SafeHarbor.538 | GGGAACATTTCTTCATAGGG | 675 |
| SafeHarbor.539 | GGTATGTGTCCATATGTGTC | 676 |
| SafeHarbor.540 | GAAGAATTAACACATTGTCT | 677 |
| SafeHarbor.541 | GATGCCTGGTTAACAATTCA | 678 |
| SafeHarbor.542 | GCCTTAAAGCTCCTATAGAA | 679 |
| SafeHarbor.543 | GGGCCCACATTTATCTCTAT | 680 |
| SafeHarbor.544 | GCAGGTGTCTAAATTCACTC | 681 |
| SafeHarbor.545 | GAACAATAAGTCAAGCAAGT | 682 |
| SafeHarbor.546 | GGGACAATCTAAATGTCCTA | 683 |
| SafeHarbor.547 | GGATATAAAAGCATACAAAA | 684 |
| SafeHarbor.548 | GAGTCACCCCAGGGACAAAC | 685 |
| SafeHarbor.549 | GGACCCTAAGGGAAGCTTGA | 686 |
| SafeHarbor.550 | GTACTCACTGATACACAGCT | 687 |
| SafeHarbor.551 | GTTTATAAATATTCCGACTA | 688 |
| SafeHarbor.552 | GGTGACTAGGAAGTTTCTGC | 689 |
| SafeHarbor.553 | GACTTAGAAACAGTTAATAA | 690 |
| SafeHarbor.554 | GTTATTATTGAGTTGGTATA | 691 |
| SafeHarbor.555 | GAACACTTTCACTGGGAATA | 692 |
| SafeHarbor.556 | GGGATTCTCCTAGAATAAAT | 693 |
| SafeHarbor.557 | GCCCACTTATGCAGTATAAG | 694 |
| SafeHarbor.558 | GTGCATACCAAATTAGTGTC | 695 |
| SafeHarbor.559 | GTATTCACAGCCAAAAAGTA | 696 |
| SafeHarbor.560 | GTTCTGCTTCTAACATAGTA | 697 |
| SafeHarbor.561 | GGAAAAGCTATGTTAAACCT | 698 |
| SafeHarbor.562 | GTATCTGCATATTAAACACA | 699 |
| SafeHarbor.563 | GGCCCTTAAAACATGGAACC | 700 |
| SafeHarbor.564 | GTAGCCTATGTCAGAATGAG | 701 |
| SafeHarbor.565 | GAGTTGCTAGACAGCTACCA | 702 |
| SafeHarbor.566 | GAAGCAACACAGATTCTCAC | 703 |
| SafeHarbor.567 | GGTTAGCAAAATTGCAAGAG | 704 |
| SafeHarbor.568 | GGAACCTGGAGAATGTTAAG | 705 |
| SafeHarbor.569 | GTGTTCTCATTCTTCACTCA | 706 |
| SafeHarbor.570 | GAGTCACGGTCAAACAGTCG | 707 |
| SafeHarbor.571 | GAGAACATACACATAATGAC | 708 |
| SafeHarbor.572 | GCTTCAAATGTGTGTGCTTC | 709 |
| SafeHarbor.573 | GAGAAATTAACTCACTTTAT | 710 |
| SafeHarbor.574 | GTATTTAGGCTATGCTTGAA | 711 |
| SafeHarbor.575 | GTCTTTGGAAACAACCATGT | 712 |
| SafeHarbor.576 | GCCCATCATGACAGGACAGG | 713 |
| SafeHarbor.577 | GGTAGAGCAGGGGTATTACT | 714 |
| SafeHarbor.578 | GGAAGTGCATGCATGACCTT | 715 |
| SafeHarbor.579 | GTTGAAATCAACATAAGGAA | 716 |
| SafeHarbor.580 | GGGGTGGCACTGGGTTAATT | 717 |
| SafeHarbor.581 | GGGCAGATCGACAACTGCCG | 718 |
| SafeHarbor.582 | GTTGAATTATGTTACCTCCA | 719 |
| SafeHarbor.583 | GAAAAATGACCCATGATTAA | 720 |
| SafeHarbor.584 | GGTAGAGGGATAATGCACTG | 721 |
| SafeHarbor.585 | GAAAGTCAAGCAGAGGGGCA | 722 |
| SafeHarbor.586 | GGAGAGAATTAATCTTATTT | 723 |
| SafeHarbor.587 | GGAGACACCAGTCACGGAGT | 724 |
| SafeHarbor.588 | GAGCCAAAGTGGCAAAGTGG | 725 |
| SafeHarbor.589 | GTGGGAGGACAGGCAGCAGA | 726 |
| SafeHarbor.590 | GATTAAAGACTTGCTTAGTT | 727 |
| SafeHarbor.591 | GAGCTTATTTGACATGTTAG | 728 |
| SafeHarbor.592 | GGATTAATGTAGCTGTAAAT | 729 |
| SafeHarbor.593 | GTAAGAGACCAAGCCCAAGT | 730 |
| SafeHarbor.594 | GGTTCACTGAGTATGTGCCC | 731 |
| SafeHarbor.595 | GGATGCAGCCACTCTCAGAG | 732 |
| SafeHarbor.596 | GAGGTACCTCACAATTTGAA | 733 |
| SafeHarbor.597 | GTATCAACAGAGTGTCAGAT | 734 |
| SafeHarbor.598 | GTACCTCAAAGTGTTCCCTG | 735 |
| SafeHarbor.599 | GGCCTCTGTAAGAGGGGAGT | 736 |
| SafeHarbor.600 | GATATATAAAGTAAGTGGAG | 737 |
| SafeHarbor.601 | GATCCTTATTGCTCCATTCT | 738 |
| SafeHarbor.602 | GAACTTATAAAGTGCCCACA | 739 |
| SafeHarbor.603 | GGTAGGGTTGGAAGGGTAAC | 740 |
| SafeHarbor.604 | GTGATGCATAGCATAGTTTC | 741 |
| SafeHarbor.605 | GGGAGGCAACCTGTCCCTGC | 742 |
| SafeHarbor.606 | GGTACAATAGATGCCTGAAA | 743 |
| SafeHarbor.607 | GGGAGTGACTCAGCTACATG | 744 |
| SafeHarbor.608 | GGTCATGATGCCACTGGGAG | 745 |
| SafeHarbor.609 | GACCAGTAAGATTAAAAATG | 746 |
| SafeHarbor.610 | GGCACTGGTTTGTGCACTTC | 747 |
| SafeHarbor.611 | GAAATATTCAAGTTTATGAG | 748 |
| SafeHarbor.612 | GTTTGCAGCACACAGGTAGA | 749 |
| SafeHarbor.613 | GTTTGGTACAGTATAACCAA | 750 |
| SafeHarbor.614 | GATCATAACAGAAGCTCCAA | 751 |
| SafeHarbor.615 | GCAAGAGCAATTCTCAGGCT | 752 |
| SafeHarbor.616 | GGGCCATGGAAAACAGCCCA | 753 |
| SafeHarbor.617 | GTGTTATGACTTTAAAGTTA | 754 |
| SafeHarbor.618 | GCAGGTCAAAAGCTCTAGAC | 755 |
| SafeHarbor.619 | GAAACCTAAACAATAGCTCC | 756 |
| SafeHarbor.620 | GCCAAGTGGACTAGAAGCCG | 757 |
| SafeHarbor.621 | GTGTCATCATGCTAAGTAAT | 758 |
| SafeHarbor.622 | GCTCTAGATTAGTTGGCTTA | 759 |
| SafeHarbor.623 | GACCTCTAATTCACAGAGAG | 760 |
| SafeHarbor.624 | GACTGAGGGTGGATAATCCA | 761 |
| SafeHarbor.625 | GAGTCGAATGTAAGAAATTC | 762 |
| SafeHarbor.626 | GATATGAGAGATAATTAAAG | 763 |
| SafeHarbor.627 | GAATACCTACCCATTAGTGA | 764 |
| SafeHarbor.628 | GTGTTAAGTAGGGAATATAC | 765 |
| SafeHarbor.629 | GAGAAATGAGGCGCTTGTTA | 766 |
| SafeHarbor.630 | GATTCACTTAGTTGCTCCCC | 767 |
| SafeHarbor.631 | GAATATGAGCTCCTAACATA | 768 |
| SafeHarbor.632 | GTACTCAGCAGAAACAAAGG | 769 |
| SafeHarbor.633 | GTGTACATAAACAAAAAGTT | 770 |
| SafeHarbor.634 | GCAGGTGCAATATTTAGTAG | 771 |
| SafeHarbor.635 | GTAAGGCCATGACACCAATT | 772 |
| SafeHarbor.636 | GTCTTAGGTGCACAATTCCC | 773 |
| SafeHarbor.637 | GTGTTATCTTTCACTCATAT | 774 |
| SafeHarbor.638 | GATTTAAGTCCTCCATGCTT | 775 |
| SafeHarbor.639 | GATTTGACATGCTTTAATAA | 776 |
| SafeHarbor.640 | GTTTCCAGGTGACTCAGTTA | 777 |
| SafeHarbor.641 | GGTCTGTGTGTGGATTTCCA | 778 |
| SafeHarbor.642 | GTCAAGCCTTATGCAATTTC | 779 |
| SafeHarbor.643 | GTCACTGGAGAAGCAACTTC | 780 |
| SafeHarbor.644 | GAGACTAAATGCGGGAAAGA | 781 |
| SafeHarbor.645 | GAACTAATCAATGTGCATCA | 782 |
| SafeHarbor.646 | GGCAGCCCTAAGGCAGTCAC | 783 |
| SafeHarbor.647 | GGGATTGTTAATGTCCAAGC | 784 |
| SafeHarbor.648 | GCATAAACATTCATGAGTTT | 785 |
| SafeHarbor.649 | GCACTCACGGAGTGCTAGGG | 786 |
| SafeHarbor.650 | GTGCTTAATATGAATGCTGG | 787 |
| SafeHarbor.651 | GGAACATGAAAATAACGTTG | 788 |
| SafeHarbor.652 | GTGACTTCATTTGATTTCAC | 789 |
| SafeHarbor.653 | GCCATCCACCATGCTATCAA | 790 |
| SafeHarbor.654 | GAGAATGGAGCTGAAAATAC | 791 |
| SafeHarbor.655 | GCTTGCTCTGTATGACTGTC | 792 |
| SafeHarbor.656 | GTCATCAGGATAAATCAGCG | 793 |
| SafeHarbor.657 | GTCTTAGTCAGGGAAGGAGT | 794 |
| SafeHarbor.658 | GGATCTCAAGAGCTACCTAA | 795 |
| SafeHarbor.659 | GAAATTACATCCCTAGATAG | 796 |
| SafeHarbor.660 | GAAGCAAAACTACCTTTGTT | 797 |
| SafeHarbor.661 | GCTTCATCTGGGGTGAAACC | 798 |
| SafeHarbor.662 | GCATTACTAACCATGGAAAG | 799 |
| SafeHarbor.663 | GTGGGTCATTCAAGTGGAGC | 800 |
| SafeHarbor.664 | GTTCCATAAGTGGAAGCGTT | 801 |
| SafeHarbor.665 | GAAATAGGAAGGGAATATAA | 802 |
| SafeHarbor.666 | GTAACACTCAGCAGCTGAGA | 803 |
| SafeHarbor.667 | GCTATTCCAGGAGAACACAT | 804 |
| SafeHarbor.668 | GTGTTGATAACAGAAGATCC | 805 |
| SafeHarbor.669 | GGATCACATATACATGCCTG | 806 |
| SafeHarbor.670 | GTCAAACTCTTCAATATTCT | 807 |
| SafeHarbor.671 | GCAACTTGAACTCCAACTTA | 808 |
| SafeHarbor.672 | GAGACTGAATATAAGATGTA | 809 |
| SafeHarbor.673 | GTGTCAAAAAACCTCAGAAA | 810 |
| SafeHarbor.674 | GTTAGGAAGTATTCGGAGTT | 811 |
| SafeHarbor.675 | GTATCAAGTAAATAGGTGGA | 812 |
| SafeHarbor.676 | GTAAAGCAACAGGTAATTAA | 813 |
| SafeHarbor.677 | GATGTTTATTGTAGGGCATG | 814 |
| SafeHarbor.678 | GACCACTCAATTTATATATT | 815 |
| SafeHarbor.679 | GGCCATTATTTGTTGATCAT | 816 |
| SafeHarbor.680 | GGAGAAACTGGATTTAAAGA | 817 |
| SafeHarbor.681 | GTCTACAGACCACAGAAGAA | 818 |
| SafeHarbor.682 | GGTATCCCTTAAGAATTTAA | 819 |
| SafeHarbor.683 | GGTAGATTAATATTCTGGAA | 820 |
| SafeHarbor.684 | GTAGTTATCCAAGGTAACAG | 821 |
| SafeHarbor.685 | GGATTTGCGCAGGTCCCTCT | 822 |
| SafeHarbor.686 | GCATGTTAGCCAGCAGAACA | 823 |
| SafeHarbor.687 | GTCACCTAAAACGATGTATG | 824 |
| SafeHarbor.688 | GATACTAATCAATAAGTGGG | 825 |
| SafeHarbor.689 | GAAGGTTATGGGAGGGGTAC | 826 |
| SafeHarbor.690 | GCAGAAAGTGATCTTTACAT | 827 |
| SafeHarbor.691 | GAAGAGGTTTAGGTTGTCAG | 828 |
| SafeHarbor.692 | GAGCCACAGTTAGAGTAACT | 829 |
| SafeHarbor.693 | GTATTGGCTAGTTAAGTGCA | 830 |
| SafeHarbor.694 | GGTCACCTTAAAAACATCTA | 831 |
| SafeHarbor.695 | GTGCATTTGGGTATTAGATT | 832 |
| SafeHarbor.696 | GAATAATAGCTATGGCTGCT | 833 |
| SafeHarbor.697 | GGGCATTGCCTGTTTAATCT | 834 |
| SafeHarbor.698 | GACTTTGTCACTAACACGCA | 835 |
| SafeHarbor.699 | GTAAGCATGTACGAAGTAAC | 836 |
| SafeHarbor.700 | GTTTGCCTTCCAGATAGGAG | 837 |
| SafeHarbor.701 | GGGAGTGTATGTTCATTGGA | 838 |
| SafeHarbor.702 | GGGTGACTACTGGTTGCTTT | 839 |
| SafeHarbor.703 | GTTAAACCTGTTTATGCTCT | 840 |
| SafeHarbor.704 | GGATTCTGAATTAATTGTAG | 841 |
| SafeHarbor.705 | GATTCTATAGTCTATAGTTA | 842 |

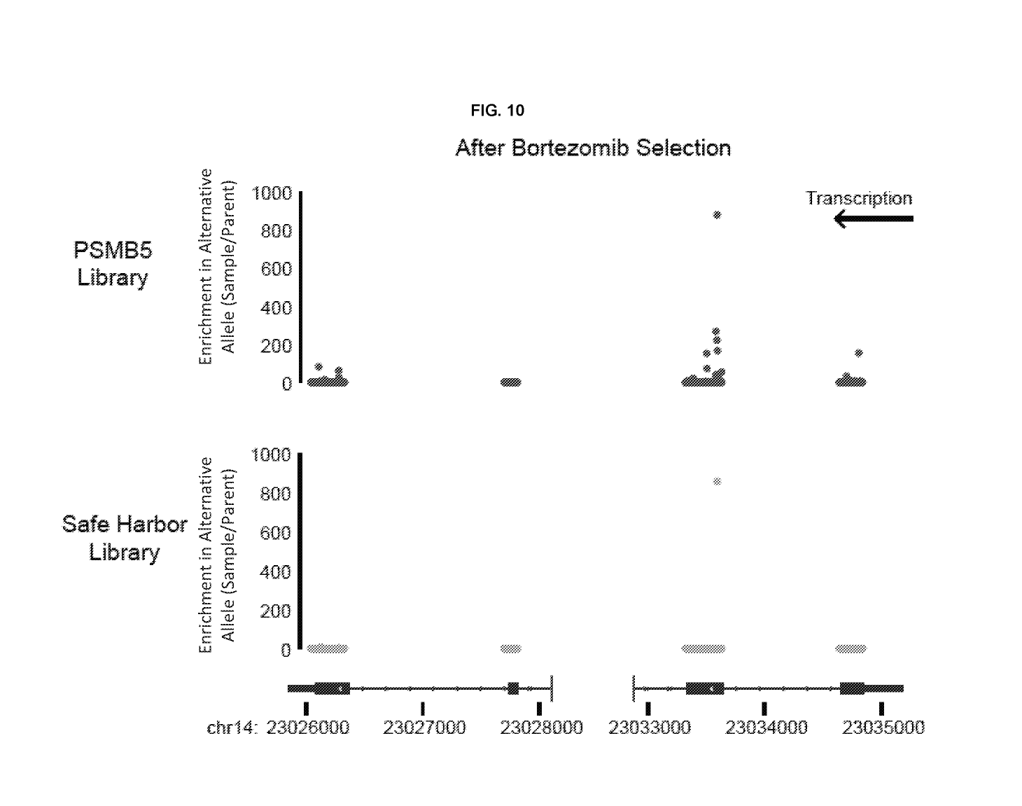

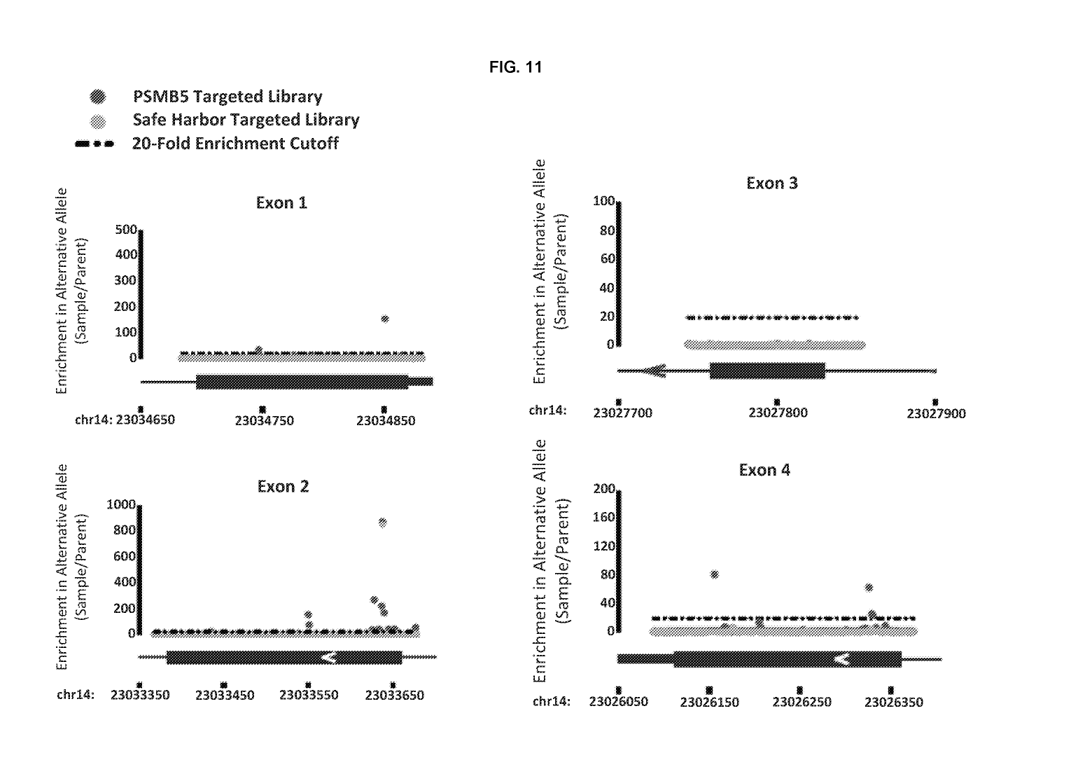

Both libraries were lentivirally integrated into K562 cells expressing dCas9 and MS2-AIDΔ, given 14 days to develop mutations, and pulsed with bortezomib three times. After selection, genomic DNA was extracted, the PSMB5 exonic loci of both libraries were sequenced, and variant frequencies were quantified at each base (FIG. 10; FIG. 11). The screen was performed in biological replicate, and mutants were selected for further analysis that showed enrichment of at least 20 fold in both replicates (FIG. 11). Eleven mutations were identified (Table 7), including two mutations (A108T/V) altering a residue known to be involved in binding bortezomib (38). Novel mutations were identified near a threonine (residue 80) that also binds bortezomib (A74V, R78M/N, A79T/G, and G82D). It is contemplated that these mutations disrupt the position of the threonine, destroying the binding pocket for bortezomib. Beyond mutations expected to affect the binding pocket, two mutations were identified in exon 1 (L11L, G45G), an intronic mutation before exon 2, and a mutation in exon 4 (G242D) that is located on the side of the protein distal to the bortezomib binding pocket. No resistant mutations were identified in exon 3, an alternate exon that is not expressed in K562 cells. In the safe harbor control library one mutation was identified (A79T) that was also found with the PSMB5 targeted library, and was likely present at undetectable levels in the parent K562 population.

TABLE 7
PSMB5 mutations and substitutions generated

| | | Amino acid |
|---|---|---|
| Genomic position | Transition | substitution |
| chr14: 23034851 | G > A | L11L |
| chr14: 23034747 | G > A | G45G |
| chr14: 23033677 | G > A | Intronic |
| chr14: 23033652 | G > A | A74V |
| chr14: 23033640 | C > A/T | R78M/N |
| chr14: 23033638 | C > T | A79T |
| chr14: 23033637 | G > C | A79G |
| chr14: 23033628 | C > T | G82D |
| chr14: 23033551 | C > T | A108T |
| chr14: 23033550 | G > A | A108V |
| chr14: 23026156 | C > T | G242D |

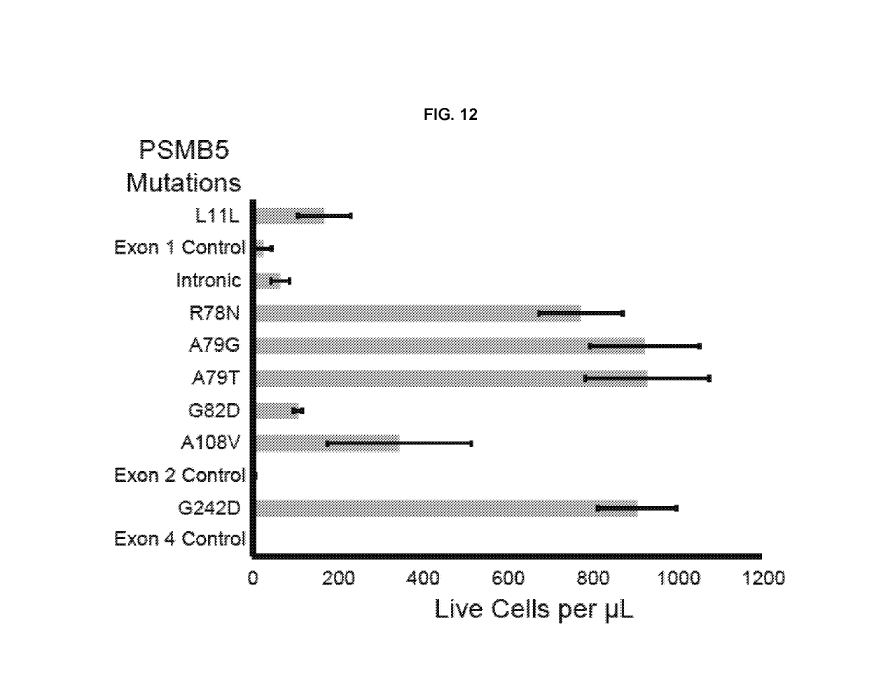

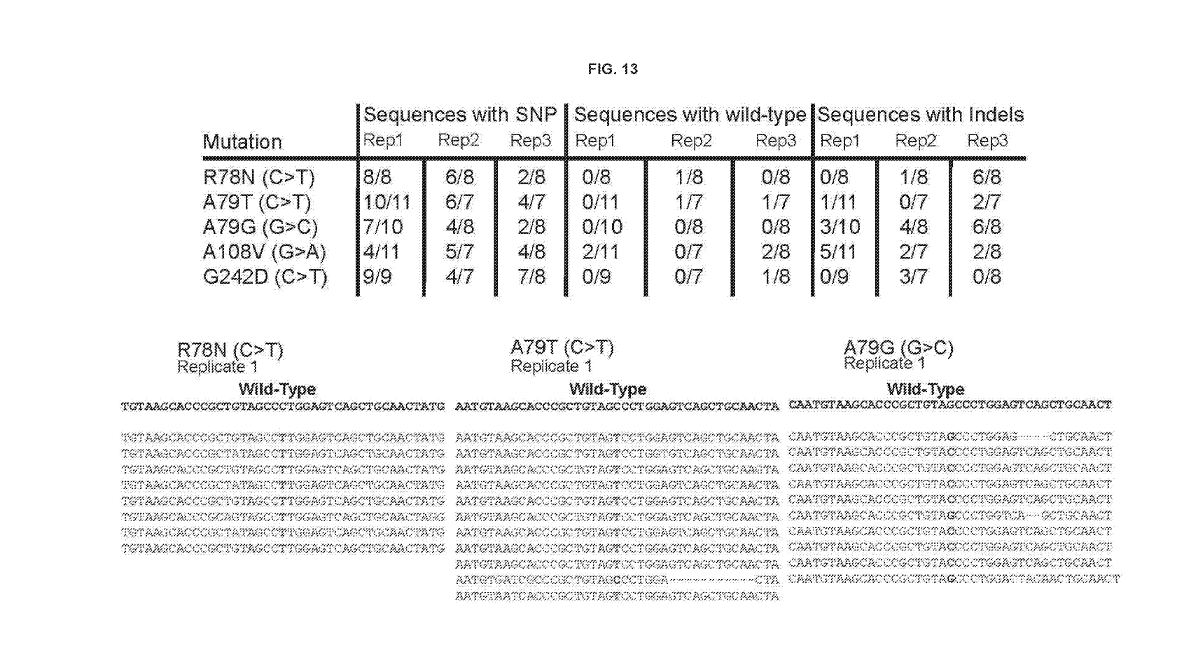

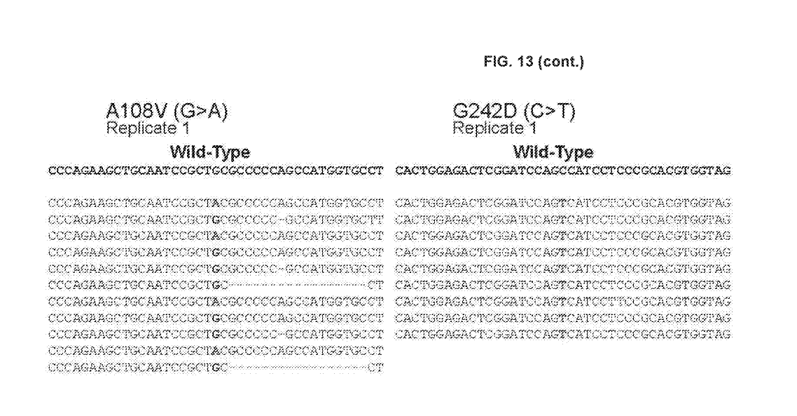

Eight of these mutations were functionally validated by knocking each one into the genome separately at the native PSMB5 locus using active Cas9 cutting followed by HDR mediated by a DNA donor oligo (26, 27). To control for the effect of Cas9 cutting and HDR, a synonymous mutation not identified in our screen was knocked into each exon. Cas9 expressing K562 cells were electroporated with donor oligo and sgRNA and incubated for six days followed by subsequent selection with bortezomib. After 14 days, the viability of the cells was measured (FIG. 12). Five of the mutations (R78N, A79G, A79T, A108V, and G242D) were strongly protective against bortezomib-induced cell death, while the other three (L11L, Intronic, and G82D) showed more modest protection when compared to controls. For the most resistant mutations, the PSMB5 locus was sequenced following bortezomib selection and the presence of the expected mutation was verified in the majority of non-frameshifted sequences (FIG. 13). Together, these experiments indicate that the technology provided herein selectively mutagenized an endogenously expressed protein target, identifying known and novel mutants that confer drug resistance.

### Example 6—Enhanced Mutagenesis Using a Hyperactive AID Mutant

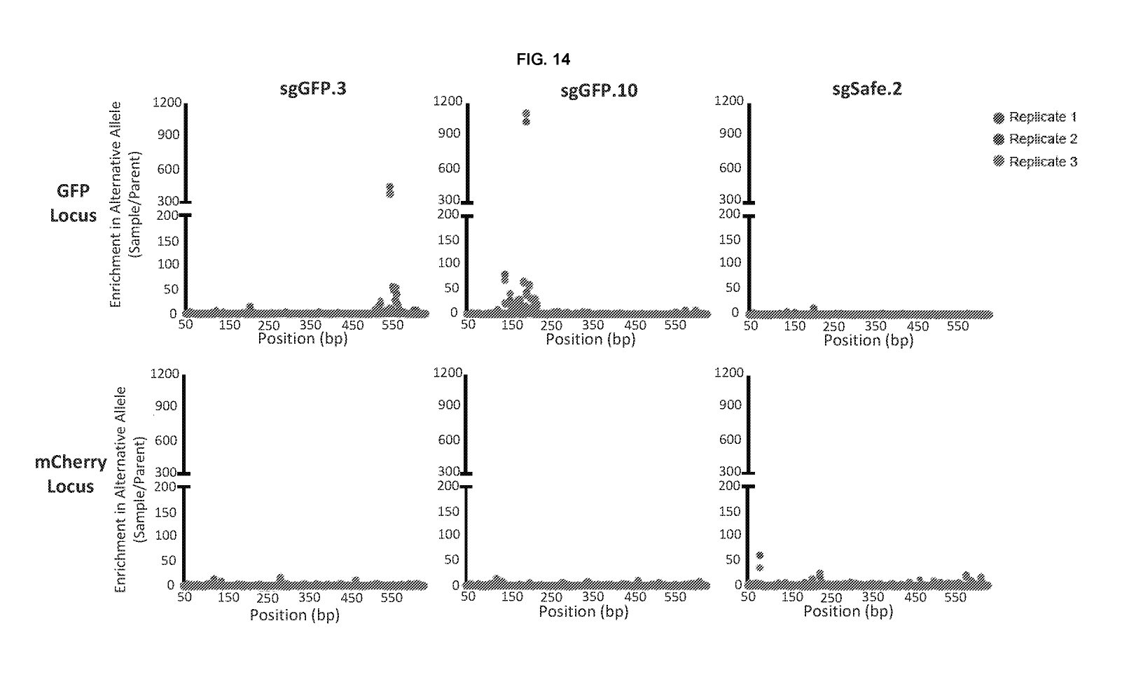

Variable mutation efficiency was observed with AIDΔ. Experiments thus investigated whether mutation efficiency improved using AID variants previously shown to have increased SHM activity (39). One of the strongest mutants (AID*) was selected and its NES was removed, similarly to removal of the NES of the wild-type AID described above (FIG. 2). This construct, AID*Δ, was integrated with one of three sgRNAs (sgGFP.3, sgGFP.10, and sgSafe.2), and enrichment of mutations in GFP and mCherry loci was measured (FIG. 14). For GFP-targeting sgRNAs, an approximate 10-fold increase in mutation was observed at the most enriched base position when compared with AIDΔ, with no noticeable increase in mCherry off-target mutation (Table 8).

TABLE 8
number of mutations per mutated sequence

| sgRNA | AIDΔ | AID*Δ |
|---|---|---|
| sgGFP.3 | 1.07 ± 0.26 | 1.31 ± 0.60 |
| sgGFP.10 | 1.07 ± 0.28 | 1.32 ± 0.61 |

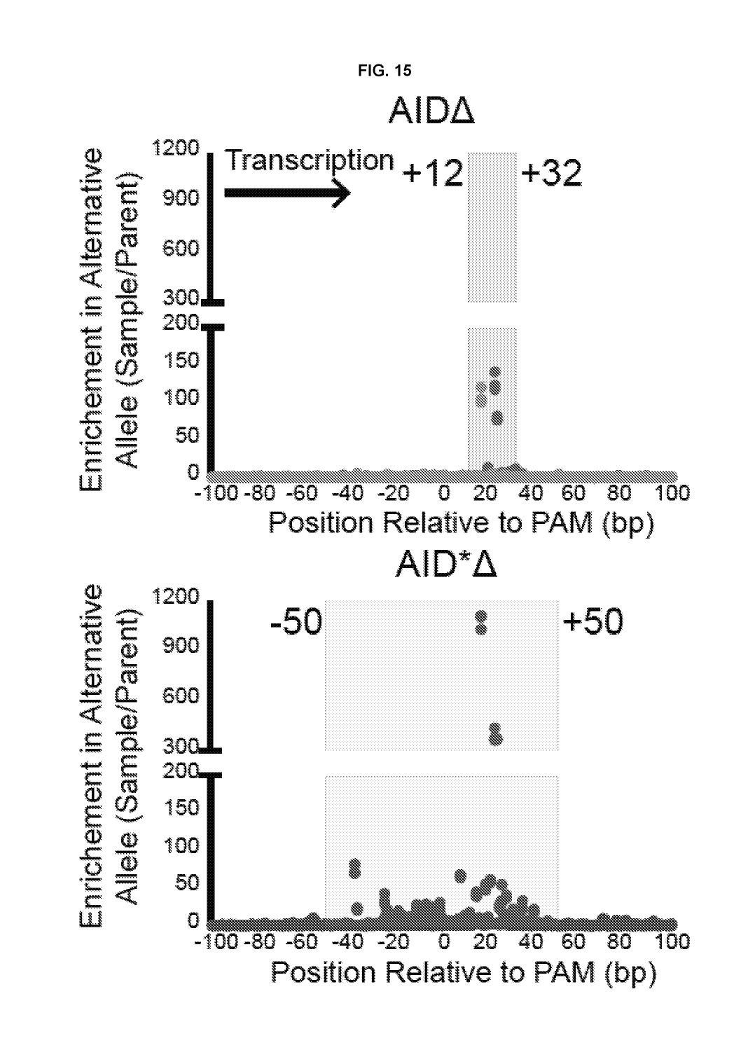

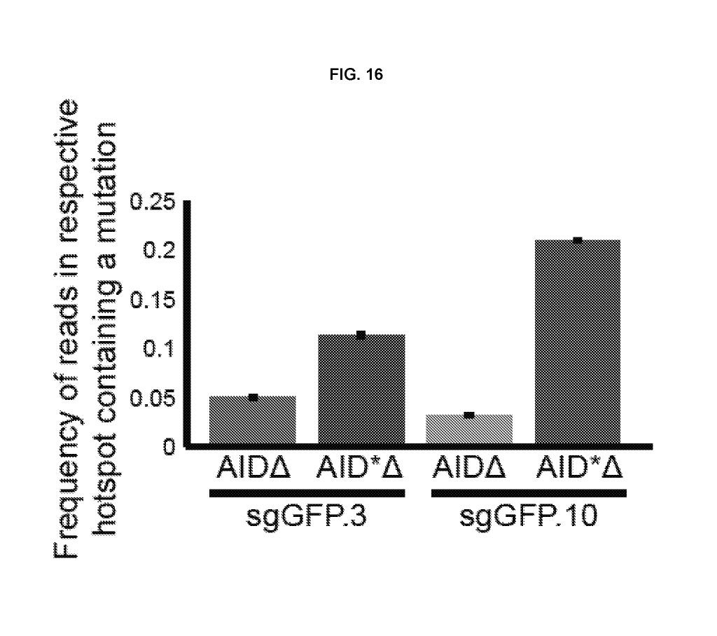

The sgSafe.2 samples did not show mutation at either locus. These mutations were aligned relative to the PAM and an increase in the size of the hotspot to span from −50 to +50 bp was observed (FIG. 15). Within this region, a substantial increase in mutation rate was observed for AID*Δ(2.25 fold for sgGFP.3 and 6.52 fold for sgGFP.10), reaching over 20% of reads for sgGFP.10 (FIG. 16), as well as an observed modest increase in sequences that contained multiple mutations per read (1.32 mutations/read for AID*Δvs. 1.07 for AIDΔ, Table 8).

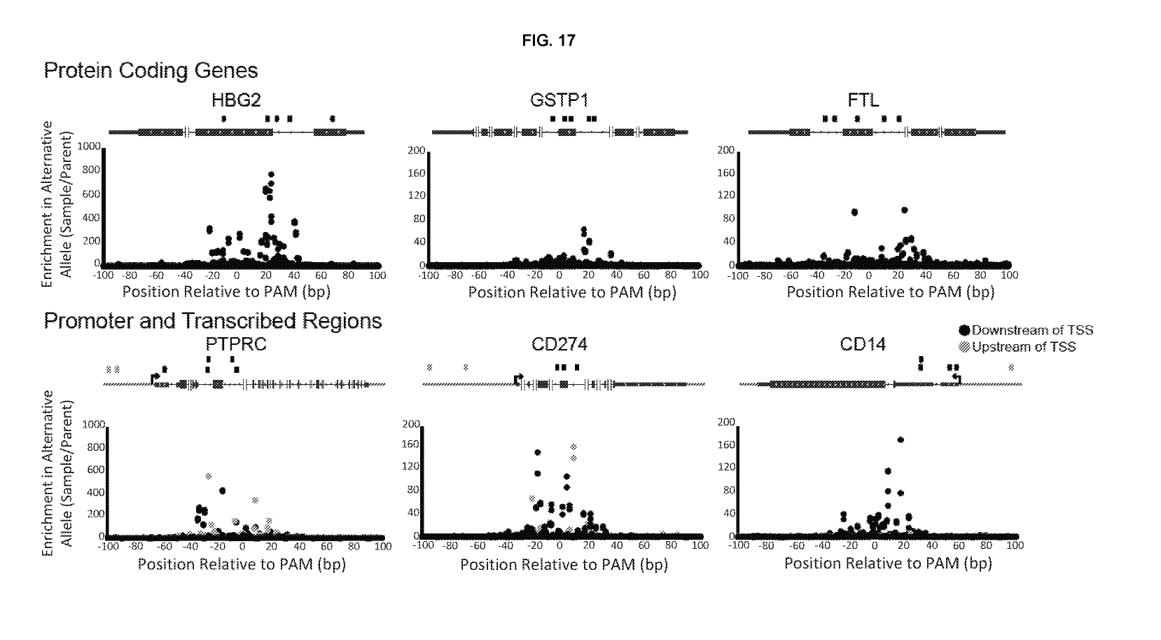

To explore further the capacity of AID*Δ-induced mutagenesis, three classes of endogenous loci were targeted: protein coding genes, promoter regions, and safe-harbor regions. For the protein coding genes, five sgRNAs were targeted to 3 highly expressed genes, FTL, HBG2, and GSTP1. The respective loci were sequenced and mutation enrichment was quantified (FIG. 17). Mutated bases were observed in each of the three genes with similar targeting in the −50 to +50 hotspot relative to the sgRNA PAM. To determine whether genes could be mutagenized with more moderate expression levels, as well as associated promoter regions, PTPRC, CD274, and CD14 were targeted. For each gene, both the transcribed region as well as sequences upstream of the transcription start site (TSS) were targeted. For each locus, mutated bases were observed for sgRNAs located both upstream and downstream of the TSS (FIG. 17). For CD274, mutations were observed up to 3.2 kb upstream of the TSS, suggesting some types of non-transcribed regions can be investigated using the technology. Lastly, sgRNAs targeting four safe harbor regions (non-functional genomic regions) were tested, but mutations were not observed in these samples.

Comparisons were made of the mutation types observed for both AIDΔ and AID*Δ within their respective hotspots. The mutation rates were normalized by alternative allele frequencies observed in the parental samples within targeted hotspot regions. In addition, the standard deviation was calculated of the alternative allele frequency in the parent samples when compared to reference sequence (5.68×10⁻⁴ for AIDΔ and 3.74×10⁻⁴ for AID*Δ), and the standard deviations were used as a noise threshold for the transition/transversion frequencies. For both AID variants, a preference for G>A and C>T transitions was observed with the most highly mutated bases being G or C, consistent with the preference of AID to exhibit deaminase activity. Furthermore, AID*Δ increases the G>A and C>T transition frequency with maximum frequencies observed at 0.211 and 0.140, respectively, compared with 0.020 and 0.016 for AIDΔ. However, the data indicated the presence of bases with alternative nucleotide frequencies above this threshold for all possible transitions and transversions except A>T for the AID*Δ treated samples. For both variants, low levels of insertions (maximum frequency of 1.98×10⁻³ for AID*Δ and 7.44×10⁻⁴ for AIDΔ) and deletions (maximum frequency of 5.15×10⁻⁴ for AID*Δ and 3.01×10⁻⁴ for AIDΔ) were observed, suggesting that mutation induced frame shifts are rare. Thus, the increased activity of AID*Δ expands the sequence space that can be mutagenized by a single sgRNA, including both coding and promoter regions of genes.

### Example 7—Simultaneous Mutation of Multiple Loci

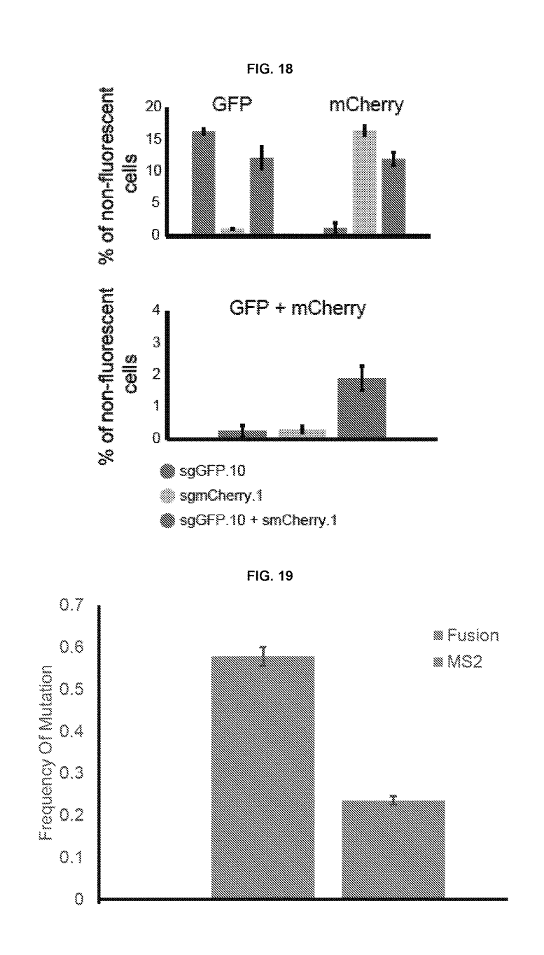

Independent mutagenesis at multiple locations is typically not possible with traditional directed evolution experiments. However, the CRISPR/Cas9 system can target multiple loci using different sgRNAs (26, 27). Accordingly, experiments were conducted using two guides, one targeting GFP (sgGFP.10) and the other targeting mCherry (sgmCherry.1), both individually and in combination. GFP and mCherry fluorescence were measured and ˜15% GFP or mCherry low populations were observed for each sgRNA individually (FIG. 18), thereby indicating that these sgRNAs were effective in generating mutations that ablated fluorescence. Upon the addition of both sgRNAs, a slight decrease in mutation of GFP or mCherry separately (˜12%) was observed, perhaps due to sharing of the mutation-generating machinery, but an increase was observed for mutations at both loci (1.92% compared to 0.26% or 0.30%) relative to cells with either sgGFP.10 or sgmCherry.1 incorporated individually. These results indicate that the technology simultaneously mutagenized two sites within the same cell, suggesting that the technology finds use in the co-evolution of more than one locus simultaneously.

### Example 8—Hyperactive AID-dCas9 Fusion

During the development of embodiments of the technology described herein, experiments were conducted to test the mutagenesis efficiency provided by fusion proteins capable of improved recruitment to target locations and/or increased mutagenesis at target locations. In particular, experiments tested alternative embodiments of the fusion proteins described herein that are capable of improved recruitment to target, that alter the mutation profile, and/or that improve efficiency. For example, data collected during these experiments indicated that a fusion protein comprising a hyperactive AID (e.g., AID*Δ as described herein) and a dCas9 produced an increased mutation rate at the target locus (e.g., in this experiment, a GFP locus). When compared to the alternative technologies (e.g., using MS2-based recruitment), the data indicated an increase in the frequency of reads comprising a mutation within the hotspot window. As shown in FIG. 19, the MS2 recruitment provided a mutation frequency of approximately 0.23 and the fusion comprising the hyperactive AID and dCas9 provided a mutation frequency of approximately 0.58.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

### REFERENCES (INCORPORATED HEREIN BY REFERENCE)

1 Doerner, A., Rhiel, L., Zielonka, S. & Kolmar, H. Therapeutic antibody engineering by high efficiency cell screening. FEBS Letters 588, 278-287 (2014).
2 Bornscheuer, U. T. et al. Engineering the third wave of biocatalysis. Nature 485, 185-194 (2012).
3 Soskine, M. & Tawfik, D. S. Mutational effects and the evolution of new protein functions. Nature Reviews. Genetics 11, 572-582 (2010).
4 Hoogenboom, H. R. Selecting and screening recombinant antibody libraries. Nature Biotechnology 23, 1105-1116 (2005).
5 Lienert, F., Lohmueller, J. J., Garg, A. & Silver, P. A. Synthetic biology in mammalian cells: next generation research tools and therapeutics. Nature Reviews. Molecular Cell Biology 15, 95-107 (2014).
6 Liu, W., Brock, A., Chen, S., Chen, S. & Schultz, P. G. Genetic incorporation of unnatural amino acids into proteins in mammalian cells. Nature Methods 4, 239-244 (2007).
7 Di Noia, J. M. & Neuberger, M. S. Molecular mechanisms of antibody somatic hypermutation. Annual Review of Biochemistry 76, 1-22 (2007).
8 Odegard, V. H. & Schatz, D. G. Targeting of somatic hypermutation. Nature Reviews. Immunology 6, 573-583 (2006).
9 Rajewsky, K., Forster, I. & Cumano, A. Evolutionary and somatic selection of the antibody repertoire in the mouse. Science 238, 1088-1094 (1987).
10 Yeap, L. S. et al. Sequence-Intrinsic Mechanisms that Target AID Mutational Outcomes on Antibody Genes. Cell 163, 1124-1137 (2015).
11 Yu, K., Huang, F. T. & Lieber, M. R. DNA substrate length and surrounding sequence affect the activation-induced deaminase activity at cytidine. The Journal of Biological Chemistry 279, 6496-6500 (2004).
12 Chaudhuri, J. et al. Transcription-targeted DNA deamination by the AID antibody diversification enzyme. Nature 422, 726-730 (2003).
13 Wang, L., Jackson, W. C., Steinbach, P. A. & Tsien, R. Y. Evolution of new nonantibody proteins via iterative somatic hypermutation. Proceedings of the National Academy of Sciences of the United States of America 101, 16745-16749 (2004).
14 Arakawa, H. et al. Protein evolution by hypermutation and selection in the B cell line DT40. Nucleic Acids Research 36, e1 (2008).
15 Bowers, P. M. et al. Coupling mammalian cell surface display with somatic hypermutation for the discovery and maturation of human antibodies. Proceedings of the National Academy of Sciences of the United States of America 108, 20455-20460 (2011).
16 Qi, L. S. et al. Repurposing CRISPR as an RNA-guided platform for sequence-specific control of gene expression. Cell 152, 1173-1183 (2013).
17 Gilbert, L. A. et al. Genome-Scale CRISPR-Mediated Control of Gene Repression and Activation. Cell 159, 647-661 (2014).
18 Konermann, S. et al. Genome-scale transcriptional activation by an engineered CRISPR-Cas9 complex. Nature 517, 583-588 (2015).
19 Chavez, A. et al. Highly efficient Cas9-mediated transcriptional programming Nature Methods 12, 326-328 (2015).
20 Ma, H. et al. Multiplexed labeling of genomic loci with dCas9 and engineered sgRNAs using CRISPRainbow. Nature Biotechnology 34, 528-530 (2016).
21 Chen, B. et al. Dynamic imaging of genomic loci in living human cells by an optimized CRISPR/Cas system. Cell 155, 1479-1491 (2013).
22 Tsai, S. Q. et al. Dimeric CRISPR RNA-guided FokI nucleases for highly specific genome editing. Nature Biotechnology 32, 569-576 (2014).
23 Kearns, N. A. et al. Functional annotation of native enhancers with a Cas9-histone demethylase fusion. Nature Methods 12, 401-403 (2015).
24 Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
25 Canver, M. C. et al. BCL11A enhancer dissection by Cas9-mediated in situ saturating mutagenesis. Nature 527, 192-197 (2015).
26 Cong, L. et al. Multiplex genome engineering using CRISPR/Cas systems. Science 339, 819-823 (2013).
27 Mali, P. et al. RNA-guided human genome engineering via Cas9. Science 339, 823-826 (2013).
28 Jinek, M. et al. A programmable dual-RNA-guided DNA endonuclease in adaptive bacterial immunity. Science 337, 816-821 (2012).
29 Findlay, G. M., Boyle, E. A., Hause, R. J., Klein, J. C. & Shendure, J. Saturation editing of genomic regions by multiplex homology-directed repair. Nature 513, 120-123 (2014).
30 Ito, S. et al. Activation-induced cytidine deaminase shuttles between nucleus and cytoplasm like apolipoprotein B mRNA editing catalytic polypeptide 1. Proceedings of the National Academy of Sciences of the United States of America 101, 1975-1980 (2004).
31 Papavasiliou, F. N. & Schatz, D. G. The activation-induced deaminase functions in a postcleavage step of the somatic hypermutation process. The Journal of Experimental Medicine 195, 1193-1198 (2002).
32 Inouye, S. & Tsuji, F. I. Evidence for redox forms of the Aequorea green fluorescent protein. FEBS letters 351, 211-214 (1994).
33 Cormack, B. P., Valdivia, R. H. & Falkow, S. FACS-optimized mutants of the green fluorescent protein (GFP). Gene 173, 33-38 (1996).
34 Tsien, R. Y. The green fluorescent protein. Annual Review of Biochemistry 67, 509-544 (1998).
35 Heim, R., Cubitt, A. B. & Tsien, R. Y. Improved green fluorescence. Nature 373, 663-664 (1995).
36 Holohan, C., Van Schaeybroeck, S., Longley, D. B. & Johnston, P. G. Cancer drug resistance: an evolving paradigm. Nature Reviews. Cancer 13, 714-726 (2013).
37 Hideshima, T. et al. The proteasome inhibitor PS-341 inhibits growth, induces apoptosis, and overcomes drug resistance in human multiple myeloma cells. Cancer Research 61, 3071-3076 (2001).
38 Lu, S. & Wang, J. The resistance mechanisms of proteasome inhibitor bortezomib. Biomarker Research 1, 13 (2013).
39 Wang, M., Yang, Z., Rada, C. & Neuberger, M. S. AID upmutants isolated using a high-throughput screen highlight the immunity/cancer balance limiting DNA deaminase activity. Nature Structural & Molecular Biology 16, 769-776 (2009).
40 Lu, S. et al. Different mutants of PSMB5 confer varying bortezomib resistance in T lymphoblastic lymphoma/leukemia cells derived from the Jurkat cell line. Experimental Hematology 37, 831-837 (2009).
41 Cancer Genome Atlas, N. Comprehensive molecular characterization of human colon and rectal cancer. Nature 487, 330-337 (2012).
42 Unniraman, S. & Schatz, D. G. AID and Igh switch region-Myc chromosomal translocations. DNA Repair 5, 1259-1264 (2006).
43 Kuppers, R., Klein, U., Hansmann, M. L. & Rajewsky, K. Cellular origin of human B-cell lymphomas. The New England Journal of Medicine 341, 1520-1529 (1999).
44 Blagodatski, A. et al. A cis-acting diversification activator both necessary and sufficient for AID-mediated hypermutation. PLoS Genetics 5, e1000332 (2009).
45 Deans, R. M. et al. Parallel shRNA and CRISPR-Cas9 screens enable antiviral drug target identification. Nature Chemical Biology 12, 361-366 (2016).
46 Hendel, A. et al. Chemically modified guide RNAs enhance CRISPR-Cas genome editing in human primary cells. Nature Biotechnology 33, 985-989 (2015).
47 Martin, M. Cutadapt removes adapter sequences from high-throughput sequencing reads. EMBnet. Journal 17, 10-12 (2011).
48 Li, H. & Durbin, R. Fast and accurate short read alignment with Burrows-Wheeler transform. Bioinformatics 25, 1754-1760 (2009).
49 Li, H. et al. The Sequence Alignment/Map format and SAMtools. Bioinformatics 25, 2078-2079 (2009).
50 Montague, T. G., Cruz, J. M., Gagnon, J. A., Church, G. M. & Valen, E. CHOPCHOP: a CRISPR/Cas9 and TALEN web tool for genome editing. Nucleic Acids Research 42, W401-407 (2014).
51 Bassik, M. C. et al. A systematic mammalian genetic interaction map reveals pathways underlying ricin susceptibility. Cell 152, 909-922 (2013).
52 Kampmann, M., Bassik, M. C. & Weissman, J. S. Integrated platform for genome-wide screening and construction of high-density genetic interaction maps in mammalian cells. Proceedings of the National Academy of Sciences of the United States of America 110, E2317-2326 (2013).
53 Bassik, M. C. et al. Rapid creation and quantitative monitoring of high coverage shRNA libraries. Nature Methods 6, 443-445 (2009).

1-78. (canceled)

79. A composition for targeted mutagenesis of a nucleic acid, the composition comprising:
a) an RNA comprising a scaffold sequence, a targeting sequence, and a binding sequence;
b) a first protein that binds to the scaffold sequence to form a RNA-guided DNA binding complex; and
c) a second protein that binds to the binding sequence and comprises a nucleic acid editing activity.

80. The composition of claim 79 wherein the RNA is an sgRNA.

81. The composition of claim 79 wherein the first protein is a dCas9.

82. The composition of claim 79 wherein the second protein comprises an MS2 protein.

83. The composition of claim 79 wherein the second protein comprises a deaminase.

84. The composition of claim 79 wherein the second protein is a hyperactive deaminase.

85. The composition of claim 79 wherein the second protein is an MS2-AID fusion protein.

86. The composition of claim 79 wherein a plurality of the second protein binds to the binding sequence.

87. The composition of claim 79 further comprising a nucleic acid comprising a target site.

88. The composition of claim 87 wherein said nucleic acid editing activity creates mutations in said nucleic acid within 20 bp to 100 bp of the target site.

89. The composition of claim 87 wherein the nucleic acid editing activity creates mutations at a rate of approximately 1 mutation per 1000 to 2000 bp.

90. A composition for simultaneous targeted mutagenesis of multiple genetic loci in the same cell, the composition comprising:
a) a first RNA comprising a scaffold sequence, a first targeting sequence, and a binding sequence;
b) a second RNA comprising said scaffold sequence, a second targeting sequence, and said binding sequence;
c) a first protein that binds to the scaffold sequence to form a RNA-guided DNA binding complex; and
d) a second protein that binds to the binding sequence and comprises a nucleic acid editing activity.

91. A method for producing a product of directed evolution, the method comprising:
a) producing a mutant pool by contacting an input nucleic acid comprising a target site to be mutagenized with a composition comprising:
1) an RNA comprising a scaffold sequence, a targeting sequence complementary to the target site, and a binding sequence;
2) a first protein that binds to the scaffold sequence to form a RNA-guided DNA binding complex; and
3) a second protein that binds to the binding sequence and comprises a nucleic acid editing activity; and
b) screening or selecting the mutant pool to identify a product of directed evolution.

92. The method of claim 91 wherein the product of directed evolution is a mutant nucleic acid comprising at least one mutation relative to the input nucleic acid.

93. The method of claim 91 wherein the product of directed evolution is a protein expressed from a mutant nucleic acid comprising at least one mutation relative to the input nucleic acid.

94. The method of claim 91 wherein the product of directed evolution is a cell or organism expressing a mutant nucleic acid comprising at least one mutation relative to the input nucleic acid or expressing a protein expressed from a mutant nucleic acid comprising at least one mutation relative to the input nucleic acid.

95. The method of claim 91 wherein the RNA, first protein, and second protein are expressed in a cell comprising the nucleic acid comprising the target site.

96. The method of claim 91 wherein the target site is a genetic locus in a genome.

97. The method of claim 91 wherein the mutant pool comprises at least 10³ to 10⁷ mutants.

98. The method of claim 91 further comprising repeating the producing and screening or selecting steps multiple times, wherein the product of directed evolution of a cycle is used to provide the input nucleic acid of a subsequent cycle.
